(19) United States
(12) Patent Application Publication
(10) Pub. No.: US 2021/0228489 A1
(43) Pub. Date: Jul. 29, 2021

(54) COMPOSITIONS FOR TREATING CYSTIC FIBROSIS

(71) Applicant: Vertex Pharmaceuticals Incorporated, Boston, MA (US)

(72) Inventors: Eleni Dokou, Waban, MA (US); Briana Lauziere, Lynnfield, MA (US); Kirk A. Overhoff, Lynn, MA (US)

(21) Appl. No.: 16/768,998

(22) PCT Filed: Dec. 4, 2018
(86) PCT No.: PCT/US2018/063871

§ 371 Date: Jun. 2, 2020

Related U.S. Application Data
(60) Provisional application No. 62/594,170, filed on Dec. 4, 2017.

(51) Int. Cl. A61K 9/20; A61K 31/4025; A61K 31/404; A61K 31/47; A61K 31/444; A61K 9/00

(52) U.S. Cl. CPC A61K 9/2059; A61K 31/4025; A61K 31/404; A61K 9/2018; A61K 9/2027; A61K 31/444; A61K 9/2054; A61K 9/0053; A61K 9/2009; A61K 31/47

(57) ABSTRACT

A single tablet comprising Compound I. Methods of treating cystic fibrosis comprising administering one or more of such single tablets to a patient.

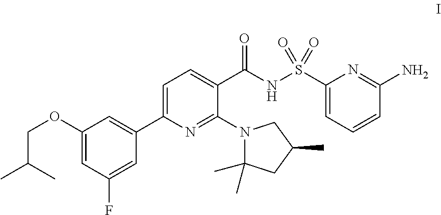 [SMILES: CC(C)COc1cc(F)cc(-c2ccc(C(=O)NS(=O)(=O)c3cccc(N)n3)c(N3C[C@@H](C)CC3(C)C)n2)c1]

This application claims the benefit of U.S. Provisional Application Disclosed 62/594,170 filed Dec. 4, 2017 and is incorporated herein in its entirety.

The application discloses pharmaceutical compositions comprising modulators of Cystic Fibrosis Transmembrane Conductance Regulator (CFTR).

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to enhanced mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 322 of these identified mutations, with sufficient evidence to define 281 mutations as disease causing. The most prevalent disease-causing mutation is a deletion of phenylalanine at position 508 of the CFTR amino acid sequence, and is commonly referred to as the F508del mutation. This mutation occurs in approximately 70% of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺-K⁺-ATPase pump and Cl— channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺-K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

Accordingly, there is a need for novel treatments of CFTR mediated diseases.

Disclosed herein are pharmaceutical compositions comprising Compound I and/or pharmaceutically acceptable salts thereof, Compound II and/or pharmaceutically acceptable salts thereof, and Compound III and/or pharmaceutically acceptable salts thereof. Compound I can be depicted as having the following structure:

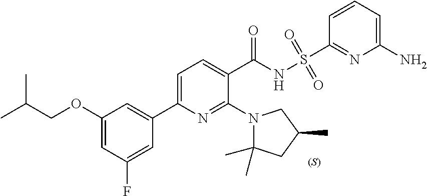

A chemical name for Compound I is N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide. PCT Publication No. WO 2016/057572, incorporated herein by reference, discloses Compound I, a method of making Compound I, and that Compound I is a CFTR modulator with an EC₃₀ of <3 μM.

Compound II can be depicted as having the following structure:

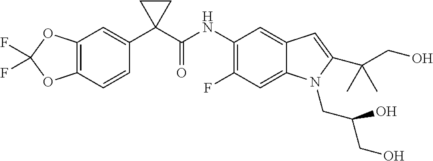 [SMILES: CC(C)(CO)c1cc2cc(NC(=O)C3(c4ccc5c(c4)OC(F)(F)O5)CC3)c(F)cc2n1C[C@@H](O)CO]

A chemical name for Compound II is (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide;

Compound III can be depicted as having the following structure:

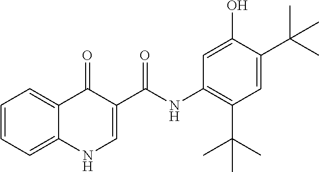 [SMILES: CC(C)(C)c1cc(C(C)(C)C)c(NC(=O)c2c[nH]c3ccccc3c2=O)cc1O]

A chemical name for Compound III is N-(5-hydroxy-2,4-di-tert-butyl-phenyl)-4-oxo-1H-quinoline-3-carboxamide.

Disclosed herein are pharmaceutical compositions wherein the properties of one therapeutic agent are improved by the presence of two therapeutic agents, kits, and methods of treatment thereof. In one embodiment, the disclosure features pharmaceutical compositions comprising N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I), (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), and N-[2,4-bis(1,1-dimethylethyl)-5-hydroxyphenyl]-1,4-dihydro-4-oxoquinoline-3-carboxamide (Compound III), wherein the composition has improved properties.

Also disclosed herein is a solid dispersion of N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) in a polymer. In one embodiment, the solid dispersion is prepared by spray drying, and is referred to a spray-dried dispersion (SDD). In one embodiment, the spray dried dispersion has a Tg of from 80° C. to 180° C. In one embodiment, Compound I in the spray dried dispersion is substantially amorphous.

### BRIEF DESCRIPTION OF THE DRAWINGS

FIG. 1 is a representative list of CFTR genetic mutations.

FIG. 2 is dissolution data for Compound I.

FIG. 3 is dissolution data for Compound II.

FIG. 4 is dissolution data for Compound III.

### DEFINITIONS

As used herein, “CFTR” means cystic fibrosis transmembrane conductance regulator.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. A genetic defect or mutation, or a change in the nucleotides in a gene in general results in a mutation in the CFTR protein translated from that gene, or a frame shift(s).

The term “F508del” refers to a mutant CFTR protein which is lacking the amino acid phenylalanine at position 508.

As used herein, a patient who is “homozygous” for a particular gene mutation has the same mutation on each allele.

As used herein, a patient who is “heterozygous” for a particular gene mutation has this mutation on one allele, and a different mutation on the other allele.

As used herein, the term “modulator” refers to a compound that increases the activity of a biological compound such as a protein. For example, a CFTR modulator is a compound that increases the activity of CFTR. The increase in activity resulting from a CFTR modulator includes but is not limited to compounds that correct, potentiate, stabilize and/or amplify CFTR.

As used herein, the term “CFTR corrector” refers to a compound that facilitates the processing and trafficking of CFTR to increase the amount of CFTR at the cell surface. Compound I, Compound II, and their pharmaceutically acceptable salts thereof disclosed herein are CFTR correctors.

As used herein, the term “CFTR potentiator” refers to a compound that increases the channel activity of CFTR protein located at the cell surface, resulting in enhanced ion transport. Compound III disclosed herein is a CFTR potentiator.

As used herein, the term “active pharmaceutical ingredient” (“API”) refers to a biologically active compound.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. Pharmaceutically acceptable salts are well known in the art. For example, S. M. Berge, et al. describe pharmaceutically acceptable salts in detail in J. Pharmaceutical Sciences, 1977, 66, 1-19.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long range order. For example, an amorphous material is a solid material having no sharp characteristic crystalline peak(s) in its X-ray power diffraction (XRPD) pattern (i.e., is not crystalline as determined by XRPD). Instead, one or more broad peaks (e.g., halos) appear in its XRPD pattern. Broad peaks are characteristic of an amorphous solid. See, e.g., US 2004/0006237 for a comparison of XRPDs of an amorphous material and crystalline material.

As used herein, the term “substantially amorphous” refers to a solid material having little or no long range order in the position of its molecules. For example, substantially amorphous materials have less than 15% crystallinity (e.g., less than 10% crystallinity or less than 5% crystallinity). It is also noted that the term ‘substantially amorphous’ includes the descriptor, ‘amorphous’, which refers to materials having no (0%) crystallinity.

As used herein, the term “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase. Or, a solid dispersion includes the drug constituting the dispersed phase, and the polymer constituting the continuous phase.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of CF or its symptoms or lessening the severity of CF or its symptoms in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The term “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### Fixed Dose Combination Single Tablets

Disclosed herein is a single tablet comprising a first solid dispersion, a second solid dispersion, and a third solid dispersion,

(a) wherein the first solid dispersion comprises 50 mg to 300 mg of Compound I:

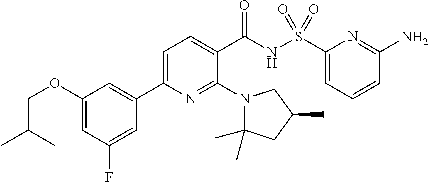 [SMILES: CC(C)COc1cc(F)cc(-c2ccc(C(=O)NS(=O)(=O)c3cccc(N)n3)c(N3C[C@@H](C)CC3(C)C)n2)c1]

and 10 wt % to 60 wt % of a polymer relative to the total weight of the first solid dispersion;

(b) wherein the second solid dispersion comprises 10 mg to 50 mg of Compound II:

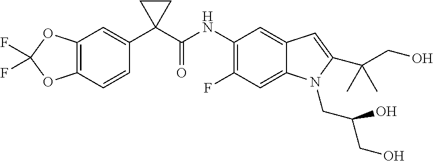 [SMILES: CC(C)(CO)c1cc2cc(NC(=O)C3(c4ccc5c(c4)OC(F)(F)O5)CC3)c(F)cc2n1C[C@@H](O)CO]

and 10 wt % to 30 wt % of a polymer relative to the total weight of the second solid dispersion; and

(c) wherein the third solid dispersion comprises 25 mg to 200 mg of Compound III:

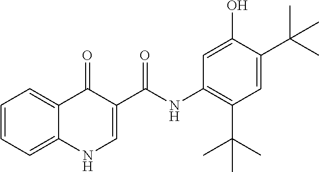 [SMILES: CC(C)(C)c1cc(C(C)(C)C)c(NC(=O)c2c[nH]c3ccccc3c2=O)cc1O]

and 10 wt % to 30 wt % of a polymer relative to the total weight of the third solid dispersion.

In some embodiments, each of Compound II and Compound III is independently substantially amorphous. In some embodiments, each of Compound II and Compound III is independently crystalline. In some embodiments, each of Compound II and Compound III is independently a mixture of forms (crystalline and/or amorphous).

### Solid Dispersions

In some embodiments, the tablets disclosed herein comprise a first solid dispersion comprising Compound I, a second solid dispersion comprising Compound II, and a third solid dispersion comprising Compound III.

In some embodiments, each of the first, second, and third solid dispersions independently comprise a plurality of particles having a mean particle diameter of 5 to 100 microns. In some embodiments, each of the first, second, and third solid dispersions independently comprise a plurality of particles having a mean particle diameter of 5 to 30 microns. In some embodiments, each of the first, second, and third solid dispersions independently comprise a plurality of particles having a mean particle diameter of 15 microns.

In some embodiments, the first solid dispersions and the first spray dried dispersions of the disclosure independently comprise substantially amorphous Compound I. In some embodiments, the second solid dispersions and the second spray dried dispersions of the disclosure independently comprises substantially amorphous Compound II. In some embodiments, the third solid dispersions and the third spray dried dispersions of the disclosure independently comprises substantially amorphous Compound III.

In some embodiments, the solid dispersions and the spray dried dispersions of the disclosure can comprise other excipients, such as polymers and/or surfactants. Any suitable polymers and surfactants known in the art can be used in the disclosure. Certain exemplary polymers and surfactants are as described below.

Solid dispersions of any one of Compounds I, II and III may be prepared by any suitable method know in the art, e.g., spray drying, lyophilizing, hot melting, or cyrogrounding/cryomilling techniques. For example, see WO2015/160787. Typically such spray drying, lyophilizing, hot melting or cyrogrounding/cryomilling techniques generates an amorphous form of API (e.g., Compound II or III).

Spray drying is a process that converts a liquid feed to a dried particulate form. Optionally, a secondary drying process such as fluidized bed drying or vacuum drying may be used to reduce residual solvents to pharmaceutically acceptable levels. Typically, spray drying involves contacting a highly dispersed liquid suspension or solution, and a sufficient volume of hot gas to produce evaporation and drying of the liquid droplets. The preparation to be spray dried can be any solution, coarse suspension, slurry, colloidal dispersion, or paste that may be atomized using the selected spray drying apparatus. In one procedure, the preparation is sprayed into a current of warm filtered gas that evaporates the solvent and conveys the dried product to a collector (e.g. a cyclone). The spent gas is then exhausted with the solvent, or alternatively the spent air is sent to a condenser to capture and potentially recycle the solvent. Commercially available types of apparatus may be used to conduct the spray drying. For example, commercial spray dryers are manufactured by Buchi Ltd. And Niro (e.g., the PSD line of spray driers manufactured by Niro) (see, US 2004/0105820; US 2003/0144257).

Techniques and methods for spray drying may be found in Perry's Chemical Engineering Handbook, 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954).

Removal of the solvent may require a subsequent drying step, such as tray drying, fluid bed drying, vacuum drying, microwave drying, rotary drum drying or biconical vacuum drying.

In one embodiment, the solid dispersions and the spray dried dispersions of the disclosure are fluid bed dried.

In one process, the solvent includes a volatile solvent, for example a solvent having a boiling point of less than 100° C. In some embodiments, the solvent includes a mixture of solvents, for example a mixture of volatile solvents or a mixture of volatile and non-volatile solvents. Where mixtures of solvents are used, the mixture can include one or more non-volatile solvents, for example, where the non-volatile solvent is present in the mixture at less than 15%, e.g., less than 12%, less than 10%, less than 8%, less than 5%, less than 3%, or less than 2%.

In some processes, solvents are those solvents where the API(s) (e.g., Compound I and/or Compound II and/or Compound III) has solubilities of at least 10 mg/ml, (e.g., at least 15 mg/ml, 20 mg/ml, 25 mg/ml, 30 mg/ml, 35 mg/ml, 40 mg/ml, 45 mg/ml, 50 mg/ml, or greater). In other processes, solvents include those solvents where the API(s) (e.g., Compound II and/or Compound III) has a solubility of at least 20 mg/ml.

Exemplary solvents that could be tested include acetone, cyclohexane, dichloromethane or methylene chloride (DCM), N,N-dimethylacetamide (DMA), N,N-dimethylformamide (DMF), 1,3-dimethyl-2-imidazolidinone (DMI), dimethyl sulfoxide (DMSO), dioxane, ethyl acetate, ethyl ether, glacial acetic acid (HAc), methyl ethyl ketone (MEK), N-methyl-2-pyrrolidinone (NMP), methyl tert-butyl ether (MTBE), tetrahydrofuran (THF), pentane, acetonitrile, methanol, ethanol, isopropyl alcohol, isopropyl acetate, and toluene. Exemplary co-solvents include DCM/methanol, acetone/DMSO, acetone/DMF, acetone/water, MEK/water, THF/water, dioxane/water. In a two solvent system, the solvents can be present in of from 0.1% to 99.9% w/w. In some preferred embodiments, water is a co-solvent with acetone where water is present from 0.1% to 15%, for example 9% to 11%, e.g., 10%. In some preferred embodiments, water is a co-solvent with MEK where water is present from 0.1% to 15%, for example 9% to 11%, e.g., 10%. In some embodiments the solvent system includes three solvents. Certain exemplary solvents include those described above, for example, MEK, DCM, water, methanol, IPA, and mixtures thereof.

The particle size and the temperature drying range may be modified to prepare an optimal solid dispersion. As would be appreciated by skilled practitioners, a small particle size would lead to improved solvent removal. Applicants have found however, that smaller particles can lead to fluffy particles that, under some circumstances do not provide optimal solid dispersions for downstream processing such as tableting.

A solid dispersion (e.g., a spray dried dispersion) disclosed herein may optionally include a surfactant. A surfactant or surfactant mixture would generally decrease the interfacial tension between the solid dispersion and an aqueous medium. An appropriate surfactant or surfactant mixture may also enhance aqueous solubility and bioavailability of the API(s) (e.g., Compound I and/or Compound II and/or Compound III) from a solid dispersion. The surfactants for use in connection with the disclosure include, but are not limited to, sorbitan fatty acid esters (e.g., Spans), polyoxyethylene sorbitan fatty acid esters (e.g., Tweens®), sodium lauryl sulfate (SLS), sodium dodecylbenzene sulfonate (SDBS) dioctyl sodium sulfosuccinate (Docusate sodium), dioxycholic acid sodium salt (DOSS), Sorbitan Monostearate, Sorbitan Tristearate, hexadecyltrimethyl ammonium bromide (HTAB), Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, Pluronic F68, Pluronic F108, and Pluronic F127 (or any other polyoxyethylene-polyoxypropylene co-polymers (Pluronics®) or saturated polyglycolized glycerides (Gelucirs®)). Specific example of such surfactants that may be used in connection with this disclosure include, but are not limited to, Span 65, Span 25, Tween 20, Capryol 90, Pluronic F108, sodium lauryl sulfate (SLS), Vitamin E TPGS, pluronics and copolymers.

In some embodiments, SLS is used as a surfactant in the solid dispersion of Compound III.

The amount of the surfactant (e.g., SLS) relative to the total weight of the solid dispersion may be between 0.1-15% w/w. For example, it is from 0.5% to 10%, such as from 0.5 to 5%, e.g., 0.5 to 4%, 0.5 to 3%, 0.5 to 2%, 0.5 to 1%, or 0.5%.

In certain embodiments, the amount of the surfactant relative to the total weight of the solid dispersion is at least 0.1% or at least 0.5%. In these embodiments, the surfactant would be present in an amount of no more than 15%, or no more than 12%, 11%, 10%, 9%, 8%, 7%, 6%, 5%, 4%, 3%, 2% or 1%. In some embodiments, the surfactant is in an amount of 0.5% by weight.

Candidate surfactants (or other components) can be tested for suitability for use in the disclosure in a manner similar to that described for testing polymers.

One aspect of the disclosure provides a method of generating a spray dried dispersion comprising (i) providing a mixture of one or more APIs and a solvent; and (ii) forcing the mixture through a nozzle and subjecting the mixture to spray drying conditions to generate the spray dried dispersion.

Another aspect of the disclosure provides a method of generating a spray dried dispersion comprising: (i) providing a mixture comprising one or more APIs and a solvent(s); and (ii) forcing the mixture out of a nozzle under spray dry drying conditions to generate a spray dried dispersion.

Another aspect of the disclosure provides a method of generating a spray dried dispersion comprising (i) spraying a mixture through a nozzle, wherein the mixture comprises one or more APIs and a solvent; and (ii) forcing the mixture through a nozzle under spray drying conditions to generate a particle that comprises the APIs.

Another aspect of the disclosure provides a spray dried dispersion comprising one or more APIs, wherein the dispersion is substantially free of a polymer, and wherein the spray dried dispersion is generated by (i) providing a mixture that consists essentially of one or more APIs and a solvent; and (ii) forcing the mixture through a nozzle under spray drying conditions to generate the spray dried dispersion.

Another aspect of the disclosure provides a spray dried dispersion comprising one or more APIs, wherein the dispersion is generated by (i) providing a mixture that comprising one or more APIs, a polymer(s), and a solvent(s); and (ii) forcing the mixture through a nozzle under spray drying conditions to generate the spray dried dispersion.

Another aspect of the disclosure provides a spray dried dispersion comprising a particle, wherein the particle comprises one or more APIs and a polymer(s), and wherein the spray dried dispersion is generated by (i) spraying a mixture through a nozzle, wherein the mixture comprises one or more APIs and a solvent; and (ii) forcing the mixture through a nozzle under spray drying conditions to generate the spray dried dispersion.

Another aspect of the disclosure provides a spray dried dispersion comprising a particle, wherein the particle comprises one or more APIs, and the particle is substantially free of a polymer, and wherein the spray dried dispersion is generated by (i) spraying a mixture through a nozzle, wherein the mixture comprises one or more APIs and a solvent; and (ii) forcing the mixture through a nozzle under spray drying conditions to generate the spray dried dispersion.

In some embodiments, the one or more APIs are selected from Compound I, Compound II, and Compound III.

Some embodiments further comprise further drying the spray dried dispersion. For example, the spray dried dispersion is dried under reduced pressure. In other examples, the spray dried dispersion is dried at a temperature of from 50° C. to 100° C.

In some embodiments, the solvent comprises a polar organic solvent. Examples of polar organic solvents include methylethyl ketone, THF, DCM, methanol, or IPA, or any combination thereof, such as, for example DCM/methanol. In other examples, the solvent further comprises water. In other examples, the solvent further comprises water. For instance, the solvent could be methylethyl ketone/water, THF/water, or methylethyl ketone/water/IPA. For example, the ratio of the polar organic solvent to water is from 70:30 to 95:5 by volume. In other instances, the ratio of the polar organic solvent to water is 90:10 by volume.

Some embodiments further comprise filtering the mixture before it is forced through the nozzle. Such filtering can be accomplished using any suitable filter media having a suitable pore size.

Some embodiments further comprise applying heat to the mixture as it enters the nozzle. This heating can be accomplished using any suitable heating element.

In some embodiments, the nozzle comprises an inlet and an outlet, and the inlet is heated to a temperature that is less than the boiling point of the solvent. For example, the inlet is heated to a temperature of from 90° C. to 150° C.

In some embodiments, the mixture is forced through the nozzle by a pressurized gas. Examples of suitable pressurized gases include those pressurized gas that are inert to the first agent, the second agent, and the solvent. In one example, the pressurized gas comprises elemental nitrogen.

In some embodiments, the pressurized gas has a positive pressure of from 90 psi to 150 psi.

Some embodiments further comprise further drying the spray dried dispersion. For example, the spray dried dispersion is dried under reduced pressure. In other examples, the spray dried dispersion is dried at a temperature of from 50° C. to 100° C.

In some embodiments, the solvent comprises a polar organic solvent. Examples of polar organic solvents include methylethyl ketone, THF, DCM, methanol, or IPA, or any combination thereof. In other examples, the solvent further comprises water. In other examples, the solvent further comprises water. For instance, the solvent could be methylethyl ketone/water, THF/water, or methylethyl ketone/water/IPA. For example, the ratio of the polar organic solvent to water is from 70:30 to 95:5 by volume. In other instances, the ratio of the polar organic solvent to water is 90:10 by volume.

In some embodiments, a pharmaceutically acceptable composition of the disclosure comprising substantially amorphous API(s) (e.g., Compound I and/or Compound II and/or Compound III) may be prepared by non-spray drying techniques, such as, for example, cyrogrounding/cryomilling techniques. A composition comprising substantially amorphous API(s) (e.g., Compound I and/or Compound II and/or Compound III) may also be prepared by hot melt extrusion techniques.

In some embodiments, the solid dispersions (e.g., spray dried dispersions) of the disclosure comprise a polymer(s). Any suitable polymers known in the art can be used in the disclosure. Exemplary suitable polymers include polymers selected from cellulose-based polymers, polyoxyethylene-based polymers, polyethylene-propylene glycol copolymers, vinyl-based polymers, PEO-polyvinyl caprolactam-based polymers, and polymethacrylate-based polymers.

The cellulose-based polymers include a methylcellulose, a hydroxypropyl methylcellulose (HPMC) (hypromellose), a hypromellose phthalate (HPMC-P), a hypromellose acetate succinate, and co-polymers thereof. The polyoxyethylene-based polymers include a polyethylene-propylene glycol, a polyethylene glycol, a poloxamer, and co-polymers thereof. The vinyl-based polymers include a polyvinylpyrrolidine (PVP), and PVP/VA. The PEO-polyvinyl caprolactam-based polymers include a polyethylene glycol, polyvinyl acetate and polyvinylcaprolactame-based graft copolymer (e.g., Soluplus®). The polymethacrylate-based polymers are synthetic cationic and anionic polymers of dimethylaminoethyl methacrylates, methacrylic acid, and methacrylic acid esters in varying ratios. Several types are commercially available and may be obtained as the dry powder, aqueous dispersion, or organic solution. Examples of such polymethacrylate-based polymers include a poly(methacrylic acid, ethyl acrylate) (1:1), a dimethylaminoethyl methacrylate-methylmethacrylate copolymer, and a Eudragit®.

In some embodiments, the cellulose-based polymer is a hypromellose acetate succinate and a hypromellose, or a combination of hypromellose acetate succinate and a hypromellose.

In some embodiments, the cellulose-based polymer is hypromellose E15, hypromellose acetate succinate L or hypromellose acetate succinate H.

In some embodiments, the polyoxyethylene-based polymer or polyethylene-propylene glycol copolymer is a polyethylene glycol or a pluronic.

In some embodiments, the polyoxyethylene-based polymer or polyethylene-propylene glycol copolymer is polyethylene glycol 3350 or poloxamer 407.

In some embodiments, the vinyl-based polymer is a vinylpolyvinylpyrrolidine-based polymer, such as polyvinylpyrrolidine K30 or polyvinylpyrrolidine VA 64.

In some embodiments, the polymethacrylate polymer is Eudragit L100-55 or Eudragit® E PO.

In some embodiments, the polymer(s) is selected from cellulosic polymers such as HPMC and/or HPMCAS.

In one embodiment, a polymer is able to dissolve in aqueous media. The solubility of the polymers may be pH independent or pH dependent. The latter include one or more enteric polymers. The term “enteric polymer” refers to a polymer that is preferentially soluble in the less acidic environment of the intestine relative to the more acid environment of the stomach, for example, a polymer that is insoluble in acidic aqueous media but soluble when the pH is above 5-6. An appropriate polymer is chemically and biologically inert. In order to improve the physical stability of the solid dispersions, the glass transition temperature (Tg) of the polymer is as high as possible. For example, polymers have a glass transition temperature at least equal to or greater than the glass transition temperature of the API. Other polymers have a glass transition temperature that is within 10 to 15° C. of the API.

Additionally, the hygroscopicity of the polymers is as low, e.g., less than 10%. For the purpose of comparison in this application, the hygroscopicity of a polymer or composition is characterized at 60% relative humidity. In some preferred embodiments, the polymer has less than 10% water absorption, for example less than 9%, less than 8%, less than 7%, less than 6%, less than 5%, less than 4%, less than 3%, or less than 2% water absorption. The hygroscopicity can also affect the physical stability of the solid dispersions. Generally, moisture adsorbed in the polymers can greatly reduce the Tg of the polymers as well as the resulting solid dispersions, which will further reduce the physical stability of the solid dispersions as described above.

In one embodiment, the polymer is one or more water-soluble polymer(s) or partially water-soluble polymer(s). Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG); polyvinyl alcohols (PVA); acrylates, such as polymethacrylate (e.g., Eudragit® E); cyclodextrins (e.g., β-cyclodextin) and copolymers and derivatives thereof, including for example PVP-VA (polyvinylpyrollidone-vinyl acetate).

In some embodiments, the polymer is hydroxypropylmethylcellulose (HPMC), such as HPMC E50, HPMC E15, or HPMC E3.

As discussed herein, the polymer can be a pH-dependent enteric polymer. Such pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S). In some embodiments, the polymer is hydroxypropyl methyl cellulose acetate succinate (HPMCAS). In some embodiments, the polymer is hydroxypropyl methyl cellulose acetate succinate HG grade (HPMCAS-HG).

In yet another embodiment, the polymer is a polyvinylpyrrolidone co-polymer, for example, avinylpyrrolidone/vinyl acetate co-polymer (PVP/VA).

In embodiments where Compound I and/or Compound II and/or Compound III forms a solid dispersion with a polymer, for example with an HPMC, HPMCAS, or PVP/VA polymer, the amount of polymer relative to the total weight of the solid dispersion ranges from 0.1% to 99% by weight. Unless otherwise specified, percentages of drug, polymer and other excipients as described within a dispersion are given in weight percentages. The amount of polymer is typically at least 20%, and preferably at least 30%, for example, at least 35%, at least 40%, at least 45%, or 50% (e.g., 49.5%). The amount is typically 99% or less, and preferably 80% or less, for example 75% or less, 70% or less, 65% or less, 60% or less, or 55% or less. In one embodiment, the polymer is in an amount of up to 50% of the total weight of the dispersion (and even more specifically, between 40% and 50%, such as 49%, 49.5%, or 50%).

In some embodiments, the API (e.g., Compound I, Compound II or Compound III) and polymer are present in roughly equal amounts in weight, for example each of the polymer and the drug make up half of the percentage weight of the dispersion. For example, the polymer is present in 49.5 wt % and Compound I, Compound II, or Compound III is present in 50 wt %. In another embodiment Compound I, Compound II, or Compound III is present in an amount greater than half of the percentage weight of the dispersions. For example, the polymer is present in 20 wt % and Compound I, Compound II, or Compound III is present in 80 wt %. In other embodiments, the polymer is present in 19.5 wt % and Compound I, Compound II, or Compound III is present in 80 wt %.

In some embodiments, the API (e.g., Compound I, Compound II, or Compound III) and the polymer combined represent 1% to 20% w/w total solid content of the spray drying solution prior to spray drying. In some embodiments, Compound I, Compound II, or Compound III, and the polymer combined represent 5% to 15% w/w total solid content of the spray drying solution prior to spray drying. In some embodiments, Compound I, Compound II, or Compound III and the polymer combined represent 11% w/w total solid content of the spray drying solution prior to spray drying.

In some embodiments, the dispersion further includes other minor ingredients, such as a surfactant (e.g., SLS). In some embodiments, the surfactant is present in less than 10% of the dispersion, for example less than 9%, less than 8%, less than 7%, less than 6%, less than 5%, less than 4%, less than 3%, less than 2%, 1%, or 0.5%.

In embodiments including a polymer, the polymer is present in an amount effective for stabilizing the solid dispersion. Stabilizing includes inhibiting or preventing, the crystallization of an API (e.g., Compound I, Compound II, or Compound III). Such stabilizing would inhibit the conversion of the API from amorphous to crystalline form. For example, the polymer would prevent at least a portion (e.g., 5%, 10%, 15%, 20%, 25%, 30%, 35%, 40%, 45%, 50%, 55%, 60%, 65%, 70%, 75%, or greater) of the API from converting from an amorphous to a crystalline form. Stabilization can be measured, for example, by measuring the glass transition temperature of the solid dispersion, measuring the amount of crystalline material, measuring the rate of relaxation of the amorphous material, or by measuring the solubility or bioavailability of the API.

In some embodiments, the polymers for use in the disclosure have a glass transition temperature of no less than 10-15° C. lower than the glass transition temperature of API. In some instances, the glass transition temperature of the polymer is greater than the glass transition temperature of API, and in general at least 50° C. higher than the desired storage temperature of the drug product. For example, at least 100° C., at least 105° C., at least 105° C., at least 110° C., at least 120° C., at least 130° C., at least 140° C., at least 150° C., at least 160° C., at least 160° C., or greater.

In some embodiments, the polymers for use in the disclosure have similar or better solubility in solvents suitable for spray drying processes relative to that of an API (e.g., Compound I, Compound II, or Compound III). In some embodiments, the polymer will dissolve in one or more of the same solvents or solvent systems as the API.

In some embodiments, the polymers for use in the disclosure can increase the solubility of an API (e.g., Compound I, Compound II, or Compound III) in aqueous and physiologically relative media either relative to the solubility of the API in the absence of polymer or relative to the solubility of the API when combined with a reference polymer. For example, the polymers can increase the solubility of Compound I, Compound II, or Compound III by reducing the amount of amorphous Compound I, Compound II, or Compound III that converts to a crystalline form(s), either from a solid amorphous dispersion or from a liquid suspension.

In some embodiments, the polymers for use in the disclosure can decrease the relaxation rate of the amorphous substance.

In some embodiments, the polymers for use in the disclosure can increase the physical and/or chemical stability of an API (e.g., Compound I, Compound II, or Compound III).

In some embodiments, the polymers for use in the disclosure can improve the manufacturability of an API (e.g., Compound I, Compound II, or Compound III).

In some embodiments, the polymers for use in the disclosure can improve one or more of the handling, administration or storage properties of an API (e.g., Compound I, Compound II, or Compound III).

In some embodiments, the polymers for use in the disclosure have little or no unfavorable interaction with other pharmaceutical components, for example excipients.

The suitability of a candidate polymer (or other component) can be tested using the spray drying methods (or other methods) described herein to form an amorphous composition. The candidate composition can be compared in terms of stability, resistance to the formation of crystals, or other properties, and compared to a reference preparation, e.g., a preparation of neat amorphous Compound I, Compound II, or Compound III. For example, a candidate composition could be tested to determine whether it inhibits the time to onset of solvent mediated crystallization, or the percent conversion at a given time under controlled conditions, by at least 50%, 75%, or 100% as well as the reference preparation, or a candidate composition could be tested to determine if it has improved bioavailability or solubility relative to crystalline Compound I, Compound II, or Compound III.

In some embodiments, the second solid dispersion comprises a cellulose polymer. For example, the first solid dispersion comprises hydroxypropyl methylcellulose (HPMC). In some embodiments, the first solid dispersion comprises a weight ratio of HPMC to Compound II ranging from 1:10 to 1:1. In some instances, the ratio of HPMC to Compound II is from 1:3 to 1:5.

In some embodiments, the third solid dispersion comprises a cellulose polymer. For example, the third solid dispersion comprises hydroxypropyl methylcellulose acetate succinate (HPMCAS).

In some embodiments, each of the second and third solid dispersions comprises a plurality of particles having a mean particle diameter of 5 to 100 microns. In some embodiments, the particles have a mean particle diameter of 5 to 30 microns. In some embodiments, the particles have a mean particle diameter of 15 microns.

In some embodiments, the second solid dispersion comprises from 70 wt % to 90 wt % (e.g., from 75 wt % to 85 wt %) of Compound II.

In some embodiments, the third solid dispersion comprises from 70 wt % to 90 wt % (e.g., from 75 wt % to 85 wt %) of Compound III.

In some embodiments, each of the second and third solid dispersions is a spray dried dispersion.

In some embodiments, the tablets disclosed herein further comprise one or more pharmaceutically acceptable excipients, such as pharmaceutically acceptable vehicles, adjuvants, or carriers.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, disclose various carriers used in formulating pharmaceutically acceptable compositions and known techniques for the preparation thereof. Except insofar as any conventional carrier medium is incompatible with the compounds of the disclosure, such as by producing any undesirable biological effect or otherwise interacting in a deleterious manner with any other component(s) of the pharmaceutically acceptable composition, its use is contemplated to be within the scope of this disclosure.

In one embodiment, the tablets of the disclosure comprise one or more fillers, a disintegrant, and a lubricant.

Fillers suitable for the tablets disclosed herein are compatible with the other ingredients of the tablets, i.e., they do not substantially reduce the solubility, the hardness, the chemical stability, the physical stability, or the biological activity of the tablets. Exemplary fillers include: celluloses, modified celluloses, (e.g. sodium carboxymethyl cellulose, ethyl cellulose hydroxymethyl cellulose, hydroxypropylcellulose), cellulose acetate, microcrystalline cellulose, calcium phosphates, dibasic calcium phosphate, starches (e.g. corn starch, potato starch), sugars (e.g., mannitol, lactose, sucrose, or the like), or any combination thereof. In one embodiment, the filler is microcrystalline cellulose.

In some embodiments, the tablets comprises one or more fillers in an amount of at least 5 wt % (e.g., at least 20 wt %, at least 30 wt %, or at least 40 wt %) by weight of the tablet. For example, the tablets comprise from 10 wt % to 60 wt % (e.g., from 20 wt % to 55 wt %, from 25 wt % to 50 wt %, or from 27 wt % to 45 wt %) of filler, by weight of the tablet. In another example, the tablets comprise at least 20 wt % (e.g., at least 30 wt % or at least 40 wt %) of microcrystalline cellulose, for example MCC Avicel PH102 or Avicel PH101, by weight of the tablet. In yet another example, the tablets comprise from 10 wt % to 60 wt % (e.g., from 20 wt % to 55 wt % or from 25 wt % to 45 wt %) of microcellulose, by weight of the tablet.

Disintegrants suitable for the tablets disclosed herein can enhance the dispersal of the tablets and are compatible with the other ingredients of the tablets, i.e., they do not substantially reduce the chemical stability, the physical stability, the hardness, or the biological activity of the tablets. Exemplary disintegrants include croscarmellose sodium, sodium starch glycolate, crospovidone or a combination thereof. In one embodiment, the disintegrant is croscarmellose sodium.

In some embodiments, the tablets disclosed herein comprise disintegrant in an amount of 10 wt % or less (e.g., 7 wt % or less, 6 wt % or less, or 5 wt % or less) by weight of the tablet. For example, the tablets comprise from 1 wt % to 10 wt % (e.g., from 1.5 wt % to 7.5 wt % or from 2.5 wt % to 6 wt %) of disintegrant, by weight of the tablet. In another example, the tablets comprise 10 wt % or less (e.g., 7 wt % or less, 6 wt % or less, or 5 wt % or less) of croscarmellose sodium, by weight of the tablet. In yet another example, the tablets comprise from 1 wt % to 10 wt % (e.g., from 1.5 wt % to 7.5 wt % or from 2.5 wt % to 6 wt %) of croscarmellose sodium, by weight of the tablet. In some examples, the tablets comprise from 0.1% to 10 wt % (e.g., from 0.5 wt % to 7.5 wt % or from 1.5 wt % to 6 wt %) of disintegrant, by weight of the tablet. In still other embodiments, the tablets comprise from 0.5% to 10 wt % (e.g., from 1.5 wt % to 7.5 wt % or from 2.5 wt % to 6 wt %) of disintegrant, by weight of the tablet.

In some embodiments, the tablets disclosed herein comprise a lubricant. A lubricant can prevent adhesion of a mixture component to a surface (e.g., a surface of a mixing bowl, a granulation roll, a compression die and/or punch). A lubricant can also reduce interparticle friction within the granulate and improve the compression and ejection of compressed pharmaceutical compositions from a granulator and/or die press. A suitable lubricant for the tablets disclosed herein is compatible with the other ingredients of the tablets, i.e., they do not substantially reduce the solubility, the hardness, or the biological activity of the tablets. Exemplary lubricants include magnesium stearate, sodium stearyl fumarate, calcium stearate, zinc stearate, sodium stearate, stearic acid, aluminum stearate, leucine, glyceryl behenate, hydrogenated vegetable oil or any combination thereof. In embodiment, the lubricant is magnesium stearate.

In one embodiment, the tablets comprise a lubricant in an amount of 5 wt % or less (e.g., 4.75 wt %, 4.0 wt % or less, or 3.00 wt % or less, or 2.0 wt % or less) by weight of the tablet. For example, the tablets comprise from 5 wt % to 0.10 wt % (e.g., from 4.5 wt % to 0.5 wt % or from 3 wt % to 1 wt %) of lubricant, by weight of the tablet. In another example, the tablets comprise 5 wt % or less (e.g., 4.0 wt % or less, 3.0 wt % or less, or 2.0 wt % or less, or 1.0 wt % or less) of magnesium stearate, by weight of the tablet. In yet another example, the tablets comprise from 5 wt % to 0.10 wt % (e.g., from 4.5 wt % to 0.15 wt % or from 3.0 wt % to 0.50 wt %) of magnesium stearate, by weight of the tablet.

Any suitable spray dried dispersions of Compound I, Compound II, and Compound III can be used for the tablets disclosed herein. Some examples for Compound II and its pharmaceutically acceptable salts can be found in WO 2011/119984 and WO 2014/015841, all of which are incorporated herein by reference. Some examples for Compound III and its pharmaceutically acceptable salts can be found in WO 2007/134279, WO 2010/019239, WO 2011/019413, WO 2012/027731, and WO 2013/130669, all of which are incorporated herein by reference.

Pharmaceutical compositions comprising Compound II and Compound III are disclosed in PCT Publication No. WO 2015/160787, incorporated herein by reference. An exemplary embodiment is shown in the following Table 2:

TABLE 2
Exemplary Tablet Comprising 100 mg of
Compound II and 150 mg of Compound III.

| | | Amount per |
|---|---|---|
| | Ingredient | tablet (mg) |
| Intra-granular | Compound II SDD (spray | 125 |
| | dried dispersion) | |
| | (80 wt % Compound II; 20 | |
| | wt % HPMC) | |
| | Compound III SDD | 187.5 |
| | (80 wt % Compound III; | |
| | 19.5 wt % HPMCAS-HG; | |
| | 0.5 wt % sodium lauryl | |
| | sulfate) | |
| | Microcrystalline cellulose | 131.4 |
| | Croscarmellose Sodium | 29.6 |
| | Total | 473.5 |
| Extra-granular | Microcrystalline cellulose | 112.5 |
| | Magnesium Stearate | 5.9 |
| | Total | 118.4 |
| Total uncoated Tablet | | 591.9 |
| Film coat | Opadry | 17.7 |
| Total coated Tablet | | 609.6 |

Pharmaceutical compositions comprising Compound III are disclosed in PCT Publication No. WO 2010/019239, incorporated herein by reference. An exemplary embodiment is shown in the following Table 3:

TABLE 3
Ingredients for Exemplary Tablet of Compound III.

| | Percent Dose | Dose | Batch |
|---|---|---|---|
| Tablet Formulation | % Wt./Wt. | (mg) | (g) |
| Compound III SDD | 34.09% | 187.5 | 23.86 |
| (80 wt % Compound III; 19.5 wt % | | | |
| HPMCAS-HG; 0.5 wt % sodium lauryl | | | |
| sulfate) | | | |
| Microcrystalline cellulose | 30.51% | 167.8 | 21.36 |
| Lactose | 30.40% | 167.2 | 21.28 |
| Sodium croscarmellose | 3.000% | 16.50 | 2.100 |
| SLS | 0.500% | 2.750 | 0.3500 |
| Colloidal silicon dioxide | 0.500% | 2.750 | 0.3500 |
| Magnesium stearate | 1.000% | 5.500 | 0.7000 |
| Total | 100% | 550 | 70 |

Additional pharmaceutical compositions comprising Compound III are disclosed in PCT Publication No. WO 2013/130669, incorporated herein by reference. Exemplary mini-tablets (˜2 mm diameter, ˜2 mm thickness, each mini-tablet weighing 6.9 mg) was formulated to have 50 mg of Compound III per 26 mini-tablets and 75 mg of Compound III per 39 mini-tablets using the amounts of ingredients recited in Table 4, below.

TABLE 4
Ingredients for mini-tablets for 50 mg and 75 mg potency

| | | Dose (mg) | Dose (mg) | |
|---|---|---|---|---|
| | Percent Dose | 50 mg | 75 mg | Batch |
| Tablet Formulation | % Wt./Wt. | potency | potency | (g) |
| Compound III SDD | 35 | 62.5 | 93.8 | 1753.4 |
| (80 wt % | | | | |
| Compound III; 19.5 | | | | |
| wt % HPMCAS- | | | | |
| HG; 0.5 wt % | | | | |
| sodium lauryl | | | | |
| sulfate) | | | | |
| Mannitol | 13.5 | 24.1 | 36.2 | 675.2 |
| Lactose | 41 | 73.2 | 109.8 | 2050.2 |
| Sucralose | 2.0 | 3.6 | 5.4 | 100.06 |
| Croscarmellose | 6.0 | 10.7 | 16.1 | 300.1 |
| sodium | | | | |
| Colloidal silicon | 1.0 | 1.8 | 2.7 | 50.0 |
| dioxide | | | | |
| Magnesium stearate | 1.5 | 2.7 | 4.0 | 74.19 |
| Total | 100 | 178.6 | 268 | 5003.15 |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 17 mg to 20 mg | |
| extragranular: | | |
| microcrystalline cellulose | 286 to 289 | mg |

| | |
|---|---|
| magnesium stearate | 5 mg to 7 mg |

In a certain embodiment, the tablets disclosed herein comprise:

| Formulation A: | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.1 | mg |
| 20 wt % hypromellose | | |
| solid dispersion containing 80 wt % Compound III, | 93.7 | mg |
| 19.5 wt % hypromellose acetate succinate, and 0.5 | | |
| wt % sodium lauryl sulfate | | |
| croscarmellose sodium (CCS) | 18.9 | mg |
| extragranular: | | |
| microcrystalline cellulose | 287.5 | mg |
| magnesium stearate | 6.3 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 18 mg to 21 mg | |
| extragranular: | | |
| microcrystalline cellulose | 108 to 111 | mg |

| | |
|---|---|
| magnesium stearate | 4 mg to 6 mg |

In a certain embodiment, the tablets disclosed herein comprise:

| Formulation B | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.2 | mg |
| 20 wt % hypromellose | | |
| solid dispersion containing 80 wt % Compound III, | 93.7 | mg |
| 19.5 wt % hypromellose acetate succinate, and 0.5 | | |
| wt % sodium lauryl sulfate | | |
| croscarmellose sodium (CCS) | 19.9 | mg |
| extragranular: | | |
| microcrystalline cellulose | 109.3 | mg |
| magnesium stearate | 4.5 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 36 mg to 39 mg | |
| extragranular: | | |
| microcrystalline cellulose | 267 to 270 | mg |

| | |
|---|---|
| magnesium stearate | 5 mg to 8 mg |

In a certain embodiment, the tablets disclosed herein comprise:

| Formulation C | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.1 | mg |
| 20 wt % hypromellose | | |
| solid dispersion containing 80 wt % Compound III, | 93.8 | mg |
| 19.5 wt % hypromellose acetate succinate, and 0.5 | | |
| wt % sodium lauryl sulfate | | |
| croscarmellose sodium (CCS) | 37.4 | mg |
| extragranular: | | |
| microcrystalline cellulose | 268.6 | mg |
| magnesium stearate | 6.2 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 27 to 30 | mg |
| extragranular: | | |
| microcrystalline cellulose | 277 to 280 | mg |

| | |
|---|---|
| magnesium stearate | 5 mg to 8 mg |

In a certain embodiment, the tablets disclosed herein comprise:

| Formulation D | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.4 | mg |
| 20 wt % hypromellose | | |
| solid dispersion containing 80 wt % Compound III, | 93.7 | mg |
| 19.5 wt % hypromellose acetate succinate, and 0.5 | | |
| wt % sodium lauryl sulfate | | |
| croscarmellose sodium (CCS) | 28.3 | mg |
| extragranular: | | |
| microcrystalline cellulose | 278.3 | mg |
| magnesium stearate | 6.3 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 22 mg to 25 mg | |
| extragranular: | | |
| microcrystalline cellulose | 273 to 276 | mg |

| | |
|---|---|
| magnesium stearate | 5 mg to 8 mg |

In a certain embodiment, the tablets disclosed herein comprise:

| Formulation E | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.2 | mg |
| 20 wt % hypromellose | | |
| solid dispersion containing 80 wt % Compound III, | 93.7 | mg |
| 19.5 wt % hypromellose acetate succinate, and 0.5 | | |
| wt % sodium lauryl sulfate | | |
| croscarmellose sodium (CCS) | 23.5 | mg |
| extragranular: | | |
| microcrystalline cellulose | 274.3 | mg |
| magnesium stearate | 6.2 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 22 to 25 | mg |
| extragranular: | | |
| microcrystalline cellulose | 178 to 181 | mg |

| | |
|---|---|
| magnesium stearate | 4 mg to 7 mg |

In a certain embodiment, the tablets disclosed herein comprise:

| Formulation F | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.2 | mg |
| 20 wt % hypromellose | | |
| solid dispersion containing 80 wt % Compound III, | 93.8 | mg |
| 19.5 wt % hypromellose acetate succinate, and 0.5 | | |
| wt % sodium lauryl sulfate | | |
| croscarmellose sodium (CCS) | 23.5 | mg |
| extragranular: | | |
| microcrystalline cellulose | 179.7 | mg |
| magnesium stearate | 5.2 | mg |

In some embodiments, the tablets disclosed herein comprise:

| | mg per tablet | |
|---|---|---|
| intragranular: | | |
| solid dispersion containing 80 wt % Compound I, | 123 to 127 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 17 to 20 | mg |
| extragranular: | | |
| microcrystalline cellulose | 142 to 145 | mg |

| | |
|---|---|
| magnesium stearate | 3 mg to 6 mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| microcrystalline cellulose | 152 to 155 | mg |
| croscarmellose sodium (CCS) | 27 mg to 30 mg | |
| extragranular: | | |
| microcrystalline cellulose | 123 to 127 | mg |

| | |
|---|---|
| magnesium stearate | 5 mg to 8 mg |

In a certain embodiment, the tablets disclosed herein comprise:

| Formulation G: | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.1 | mg |
| 20 wt % hypromellose | | |
| solid dispersion containing 80 wt % Compound III, | 93.8 | mg |
| 19.5 wt % hypromellose acetate succinate, and 0.5 | | |
| wt % sodium lauryl sulfate | | |
| microcrystalline cellulose | 153.0 | mg |
| croscarmellose sodium (CCS) | 28.1 | mg |
| extragranular: | | |
| microcrystalline cellulose | 124.9 | mg |
| magnesium stearate | 6.2 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 29 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| microcrystalline cellulose | 152 mg to 155 mg | |
| extragranular: | | |
| microcrystalline cellulose | 123 to 127 | mg |
| magnesium stearate | 5 mg to 8 mg | |
| croscarmellose sodium | 27 to 30 | mg |

In a certain embodiment, the tablets disclosed herein comprise:

| Formulation H | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.2 | mg |
| 20 wt % hypromellose | | |
| solid dispersion containing 80 wt % Compound III, | 93.5 | mg |
| 19.5 wt % hypromellose acetate succinate, and 0.5 | | |
| wt % sodium lauryl sulfate | | |
| microcrystalline cellulose | 153.1 | mg |
| extragranular: | | |
| microcrystalline cellulose | 124.9 | mg |
| magnesium stearate | 6.2 | mg |
| croscarmellose sodium | 28.1 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 29 to 33 | mg |
| 20 wt % hypromellose | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| croscarmellose sodium (CCS) | 36 to 39 | mg |
| sodium lauryl sulfate | 5 to 8 | mg |
| extragranular: | | |
| microcrystalline cellulose | 261 to 264 | mg |

| | |
|---|---|
| magnesium stearate | 5 mg to 8 mg |

In a certain embodiment, the tablets disclosed herein comprise:

| Formulation I | mg per tablet | |
|---|---|---|
| intragranular: | | |
| solid dispersion containing 80 wt % Compound I, | 187.5 | mg |
| 20 wt % hypromellose acetate succinate | | |
| solid dispersion containing 80 wt % Compound II, | 31.4 | mg |
| 20 wt % hypromellose | | |
| solid dispersion containing 80 wt % Compound III, | 93.8 | mg |
| 19.5 wt % hypromellose acetate succinate, and 0.5 | | |
| wt % sodium lauryl sulfate | | |
| croscarmellose sodium (CCS) | 37.4 | mg |
| sodium lauryl sulfate | 6.4 | mg |
| extragranular: | | |
| microcrystalline cellulose | 262.7 | mg |
| magnesium stearate | 6.3 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| microcrystalline cellulose | 136 mg to 139 mg | |
| croscarmellose sodium | 27 to 30 | mg |
| extragranular: | | |
| solid dispersion containing 80 wt % Compound III, | 93 mg to 96 mg | |
| 19.5 wt % hypromellose acetate succinate, and 0.5 | | |
| wt % sodium lauryl sulfate | | |
| microcrystalline cellulose | 375 to 378 | mg |

In some embodiments, the tablets disclosed herein comprise:

| | mg per tablet | |
|---|---|---|
| intragranular: | | |
| solid dispersion containing 80 wt % Compound I, | 186 to 189 | mg |
| 20 wt % hypromellose acetate succinate | | |
| solid dispersion containing 80 wt % Compound II, | 30 to 33 | mg |
| 20 wt % hypromellose | | |
| microcrystalline cellulose | 136 to 139 | mg |
| croscarmellose sodium (CCS) | 27 to 30 | mg |
| extragranular: | | |
| solid dispersion containing 80 wt % Compound III, | 92 mg to 95 mg | |
| 19.5 wt % hypromellose acetate succinate, and 0.5 | | |
| wt % sodium lauryl sulfate | | |
| microcrystalline cellulose | 280 to 283 | mg |

In some embodiments, the tablets disclosed herein comprise:

| | mg per tablet | |
|---|---|---|
| intragranular: | | |
| solid dispersion containing 80 wt % Compound II, | 30 to 33 | mg |
| 20 wt % hypromellose | | |
| solid dispersion containing 80 wt % Compound III, | 92 mg to 95 | mg |
| 19.5 wt % hypromellose acetate succinate, and 0.5 | | |
| wt % sodium lauryl sulfate | | |
| croscarmellose sodium (CCS) | 36 to 39 | mg |
| microcrystalline cellulose | 142 to 145 | mg |
| extragranular: | | |
| solid dispersion containing 80 wt % Compound I, | 186 to 189 | mg |
| 20 wt % hypromellose acetate succinate | | |
| microcrystalline cellulose | 123 to 127 | mg |
| magnesium stearate | 5 to 8 | mg |

In a certain embodiment, the tablets disclosed herein comprise:

| Formulation J | mg per tablet | |
|---|---|---|
| intragranular: | | |
| solid dispersion containing 80 wt % Compound II, | 31.2 | mg |
| 20 wt % hypromellose | | |
| solid dispersion containing 80 wt % Compound III, | 93.7 | mg |
| 19.5 wt % hypromellose acetate succinate, and 0.5 | | |
| wt % sodium lauryl sulfate | | |
| croscarmellose sodium (CCS) | 37.4 | mg |
| microcrystalline cellulose | 143.6 | mg |
| extragranular: | | |
| solid dispersion containing 80 wt % Compound I, | 187.5 | mg |
| 20 wt % hypromellose acetate succinate | | |
| microcrystalline cellulose | 125 | mg |
| magnesium stearate | 6.3 | mg |

In some embodiments, the tablets disclosed herein comprise:

| | mg per tablet | |
|---|---|---|
| intragranular: | | |
| solid dispersion containing 80 wt % Compound II, | 30 to 33 | mg |
| 20 wt % hypromellose | | |
| solid dispersion containing 80 wt % Compound III, | 92 mg to 95 | mg |
| 19.5 wt % hypromellose acetate succinate, and 0.5 | | |
| wt % sodium lauryl sulfate | | |
| croscarmellose sodium (CCS) | 36 to 39 | mg |
| extragranular: | | |
| solid dispersion containing 80 wt % Compound I, | 186 to 189 | mg |
| 20 wt % hypromellose acetate succinate | | |
| microcrystalline cellulose | 267 to 270 | mg |
| magnesium stearate | 5 to 8 | mg |

### Processes of Making Tablets

The tablets of the disclosure can be produced by compacting or compressing an admixture or composition, for example, powder or granules, under pressure to form a stable three-dimensional shape (e.g., a tablet). As used herein, “tablet” includes compressed pharmaceutical dosage unit forms of all shapes and sizes, whether coated or uncoated. In some embodiments, the methods of preparing the tablets disclosed herein comprise (a) mixing the first, second, and third solid dispersions to form a first mixture; and (b) compressing a tablet mixture comprising the first mixture into a tablet. As used herein, the term “mixing” include mixing, blending and combining. In some embodiments, the tablet mixture further comprises one or more pharmaceutically acceptable excipients, and the methods further comprise mixing the first mixture with said one or more excipients to form the tablet mixture. Mixing the first mixture with one or more excipients can be performed in one or more steps. In one embodiment, the one or more excipients are mixed to form a second mixture; and the first and second mixtures are mixed together to form the tablet mixture prior to the compression step. In one embodiment, the one or more excipients can be mixed with the first mixture in more than one parts, for example, some excipients mixed with the first mixture first and the other excipients followed later. In some embodiments, the tablets disclosed herein an intra-granular part and an extra-grandular part as described above, and one or more excipients included in the intra-granular part are mixed to form a second mixture, and one or more excipients included in the extra-granular part are mixed to form a third mixture, and the first mixture are combined with the second mixture, and the combined first and second mixtures are combined with the third mixture to form a tablet mixture.

In some embodiments, the methods of preparing the tablets disclosed herein comprise: (a) mixing the first, second, and third solid dispersions to form a first mixture; (b) mixing the first mixture with one or more of microcrystalline cellulose, croscarmellose sodium and magnesium stearate to form a tablet mixture; and (c) compressing the tablet mixture into a tablet.

In some embodiments, the methods of preparing the tablets disclosed herein comprise:

(a) mixing the first, second, and third solid dispersions described above to form a first mixture; (b) mixing one or more of microcrystalline cellulose, croscarmellose sodium and magnesium stearate in an intra-granular part to form a second mixture; (c) mixing one or more of microcrystalline cellulose, croscarmellose sodium, and magnesium stearate in an extra-granular part to form a third mixture; (d) mixing the first, second, and third mixtures to form a tablet mixture; and (e) compressing the tablet mixture comprising the first, second and third mixtures into a tablet. It is noted that step (a) can occur prior to step (b) or step (b) can occur prior to step (a).

In some embodiments, the methods disclosed herein further comprise coating the tablet.

In some embodiments, the methods disclosed herein further comprise granulating the first, second, and/or third mixtures prior to the compression the tablet mixture. Any suitable methods known in the art for granulation and compression of pharmaceutical compositions can be used. It is noted that step (a) can occur prior to step (b) or step (b) can occur prior to step (a).

### Granulation and Compression

In some embodiments, solid forms, including powders comprising one or more APIs (e.g., Compound I, Compound II, and/or Compound III) and the included pharmaceutically acceptable excipients (e.g. filler, diluent, disintegrant, surfactant, glidant, binder, lubricant, or any combination thereof) can be subjected to a dry granulation process. The dry granulation process causes the powder to agglomerate into larger particles having a size suitable for further processing. Dry granulation can improve the flowability of a mixture to produce tablets that comply with the demand of mass variation or content uniformity.

In some embodiments, formulations can be produced using one or more mixing and dry granulations steps. The order and the number of the mixing by granulation. At least one of the excipients and the API(s) can be subject to dry granulation or wet high shear granulation or twin screw wet granulation before compression into tablets. Dry granulation can be carried out by a mechanical process, which transfers energy to the mixture without any use of any liquid substances (neither in the form of aqueous solutions, solutions based on organic solutes, or mixtures thereof) in contrast to wet granulation processes, also contemplated herein. Generally, the mechanical process requires compaction such as the one provided by roller compaction. An example of an alternative method for dry granulation is slugging. In some embodiments, wet granulations instead of the dry granulation can be used.

In some embodiments, roller compaction is a granulation process comprising mechanical compacting of one or more substances. In some embodiments, a pharmaceutical composition comprising an admixture of powders is pressed, that is roller compacted, between two rotating rollers to make a solid sheet that is subsequently crushed in a sieve to form a particulate matter. In this particulate matter, a close mechanical contact between the ingredients can be obtained. An example of roller compaction equipment is Minipactor® a Gerteis 3W-Polygran from Gerteis Maschinen+Processengineering AG.

In some embodiments, tablet compression according to the disclosure can occur without any use of any liquid substances (neither in the form of aqueous solutions, solutions based on organic solutes, or mixtures thereof), i.e., a dry granulation process. In a typical embodiment the resulting core or tablet has a tensile strength in the range of from 0.5 MPa to 3.0 MPa; such as 1.0 to 2.5 MPa, such as in the range of 1.5 to 2.0 MPa.

In some embodiments, the ingredients are weighed according to the formula set herein. Next, all of the intragranular ingredients are sifted and mixed well. The ingredients can be lubricated with a suitable lubricant, for example, magnesium stearate. The next step can comprise compaction/slugging of the powder admixture and sized ingredients. Next, the compacted or slugged blends are milled into granules and may optionally be sifted to obtain the desired size. Next, the granules can be further blended or lubricated with, for example, magnesium stearate. Next, the granular composition of the disclosure can be compressed on suitable punches into various pharmaceutical formulations in accordance with the disclosure. Optionally the tablets can be coated with a film coat.

Another aspect of the disclosure provides a method for producing a pharmaceutical composition comprising an admixture of a composition comprising one or more APIs (e.g., Compound I, Compound II and/or Compound III); and one or more excipients selected from: one or more fillers, a diluent, a binder, a glidant, a surfactant, a lubricant, a disintegrant, and compressing the composition into a tablet.

### Coating

In some embodiments, the tablets disclosed herein can be coated with a film coating and optionally labeled with a logo, other image and/or text using a suitable ink. In still other embodiments, the tablets disclosed herein can be coated with a film coating, waxed, and optionally labeled with a logo, other image and/or text using a suitable ink. Suitable film coatings and inks are compatible with the other ingredients of the tablets, e.g., they do not substantially reduce the solubility, the chemical stability, the physical stability, the hardness, or the biological activity of the tablets. The suitable colorants and inks can be any color and are water based or solvent based. In one embodiment, the tablets disclosed herein are coated with a colorant and then labeled with a logo, other image, and/or text using a suitable ink.

In some embodiments, the tablets disclosed herein are coated with a film that comprises 2-6 wt % by the weight of the uncoated tablet. In some embodiments, the film comprises one or more colorants and/or pigments. In some embodiments, the tablets disclosed herein are coated with a film that comprises one or more colorants and/or pigments and wherein the film comprises 2-5 wt % by the weight of the uncoated tablet. In some embodiments, the tablets disclosed herein are coated with a film that comprises one or more colorants and/or pigments and wherein the film comprises 2-4 wt % by the weight of the uncoated tablet. The colored tablets can be labeled with a logo and text indicating the strength of the active ingredient in the tablet using a suitable ink.

### Methods of Treatment

The tablets disclosed herein can be administered once a day, twice a day, or three times a day. In some embodiments, one or more of the tablets are administered per dosing. In some embodiments, two tablets per dosing are administered. In some embodiments, two tablets per dosing are administered twice a day. An effective amount of the APIs (e.g., Compound (I)) is administered to the patient with or using one or more tablets disclosed herein.

The tablets disclosed herein are useful for treating cystic fibrosis.

In some aspects, the tablets disclosed herein can be employed in combination therapies. In some embodiments, the tablets disclosed herein can be administered concurrently with, prior to, or subsequent to, at least one active pharmaceutical ingredients or medical procedures.

In some embodiments, the pharmaceutical compositions are a tablet. In some embodiments, the tablets are suitable for oral administration.

The tablets disclosed herein, optionally with additional active pharmaceutical ingredients or medical procedures are useful for treating cystic fibrosis in a patient.

In some embodiments, disclosed herein is a method of treating, lessening the severity of, or symptomatically treating cystic fibrosis in a patient comprising administering one or more tablets of this disclosure to the patient, such as a human, wherein said patient has cystic fibrosis. In some embodiments, the patient is chosen from patients with F508del/minimal function (MF) genotypes, patients with F508del/F508del genotypes, patients with F508del/gating genotypes, and patients with F508del/residual function (RF) genotypes.

In some embodiments, the patient is heterozygous for F508del, and the other CFTR genetic mutation is any CF-causing mutation, and is expected to be and/or is responsive to any combinations of (i) Compound I, and (ii) Compound II, and/or Compound III and/or Compound IV genotypes based on in vitro and/or clinical data.

Compounds I, II, and III are as depicted above. Compound IV is depicted as having the following structure:

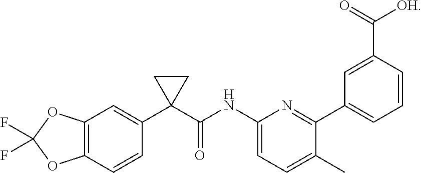 [SMILES: Cc1ccc(NC(=O)C2(c3ccc4c(c3)OC(F)(F)O4)CC2)nc1-c1cccc(C(=O)O)c1]

A chemical name for Compound IV is 3-(6-(1-(2,2-difluorobenzo[d][1,3]dioxol-5-yl)cyclopropanecarboxamido)-3-methylpyridin-2-yl)benzoic acid. Patients with an F508del/minimal function genotype are defined as patients that are heterozygous F508del-CFTR with a second CFTR allele containing a mutation that is predicted to result in a CFTR protein with minimal function and that is not expected to respond to Compound II, Compound III, or the combination of Compound II and Compound III. These CFTR mutations were defined using 3 major sources:
biological plausibility for the mutation to respond (i.e., mutation class)
evidence of clinical severity on a population basis (per CFTR2 patient registry; accessed on 15 Feb. 2016)
   average sweat chloride>86 mmol/L, and
   prevalence of pancreatic insufficiency (PI)>50%
in vitro testing
   mutations resulting in baseline chloride transport<10% of wild-type CFTR were considered minimal function
   mutations resulting in chloride transport<10% of wild-type CFTR following the addition of Compound II and/or Compound III were considered nonresponsive.

In some embodiments, disclosed herein is a method of treating, lessening the severity of, or symptomatically treating cystic fibrosis in a patient comprising administering one or more tablets of this disclosure to the patient, such as a human, wherein the patient possesses a CFTR genetic mutation G551D. In some embodiments, the patient is homozygous for the G551D genetic mutation. In some embodiments, the patient is heterozygous for the G551D genetic mutation. In some embodiments, the patient is heterozygous for the G551D genetic mutation, having the G551D mutation on one allele and any other CF-causing mutation on the other allele. In some embodiments, the patient is heterozygous for the G551D genetic mutation on one allele and the other CF-causing genetic mutation on the other allele is any one of F508del, G542X, N1303K, W1282X, R117H, R553X, 1717-1G->A, 621+1G->T, 2789+5G->A, 3849+10kbC->T, R1162X, G85E, 3120+1G->A, ΔI507, 1898+1G->A, 3659delC, R347P, R560T, R334W, A455E, 2184delA, or 711+1G->T. In some embodiments, the patient is heterozygous for the G551D genetic mutation, and the other CFTR genetic mutation is F508del. In some embodiments, the patient is heterozygous for the G551D genetic mutation, and the other CFTR genetic mutation is R117H.

In some embodiments, disclosed herein is a method of treating, lessening the severity of, or symptomatically treating cystic fibrosis in a patient, such as a mammal, wherein the patient possesses a CFTR genetic mutation F508del. In some embodiments, the patient is homozygous for the F508del genetic mutation. In some embodiments, the patient is heterozygous for the F508del genetic mutation wherein the patient has the F508del genetic mutation on one allele and any CF-causing genetic mutation on the other allele. In some embodiments, the patient is heterozygous for F508del, and the other CFTR genetic mutation is any CF-causing mutation, including, but not limited to G551D, G542X, N1303K, W1282X, R117H, R553X, 1717-1G->A, 621+1G->T, 2789+5G->A, 3849+10kbC->T, R1162X, G85E, 3120+1G->A, ΔI507, 1898+1G->A, 3659delC, R347P, R560T, R334W, A455E, 2184delA, or 711+1G->T. In some embodiments, the patient is heterozygous for F508del, and the other CFTR genetic mutation is G551D. In some embodiments, the patient is heterozygous for F508del, and the other CFTR genetic mutation is R117H.

In some embodiments, disclosed herein is a method of treating, lessening the severity of, or symptomatically treating cystic fibrosis in a patient comprising administering an effective amount of a tablet of this disclosure to the patient, such as a mammal, wherein the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, I1005R, I1234V, I1269N, I1366N, I175V, I502T, I506S, I506T, I601F, I618T, 1807M, 1980K, L102R, L1324P, L1335P, L138ins, L1480P, L15P, L165S, L320V, L346P, L453S, L571S, L967S, M1101R, M152V, M1T, M1V, M265R, M952I, M952T, P574H, P5L, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T1053I, T1246I, T604I, V1153E, V1240G, V1293G, V201M, V232D, V456A, V456F, V562I, W1098C, W1098R, W1282R, W361R, W57G, W57R, Y1014C, Y1032C, Y109N, Y161D, Y161S, Y563D, Y563N, Y569C, and Y913C. In some embodiments, the patient has at least one combination mutation chosen from: G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, and 621+3A->G.

In some embodiments, the patient has at least one combination mutation chosen from: 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, I1005R, I1234V, I1269N, I1366N, I175V, I502T, I506S, I506T, I601F, I618T, I807M, I980K, L102R, L1324P, L1335P, L138ins, L1480P, L15P, L165S, L320V, L346P, L453S, L571S, L967S, M1101R, M152V, M1T, M1V, M265R, M952I, M952T, P574H, P5L, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T1053I, T1246I, T604I, V1153E, V1240G, V1293G, V201M, V232D, V456A, V456F, V562I, W1098C, W1098R, W1282R, W361R, W57G, W57R, Y1014C, Y1032C, Y109N, Y161D, Y161S, Y563D, Y563N, Y569C, and Y913C.

In some embodiments, the patient has at least one combination mutation chosen from:

### D443Y; G576A; R668C,

### F508C; S1251N,

### G576A; R668C,

### G970R; M470V,

### R74W; D1270N,

### R74W; V201M, and

### R74W; V201M; D1270N.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V and G1069R. In some embodiments, this disclosure provides a method of treating CFTR comprising administering a tablet disclosed herein a patient possessing a human CFTR mutation selected from G178R, G551S, G970R, G1244E, S1255P, G1349D, S549N, S549R and S1251N. In some embodiments, disclosed herein is a method of treating, lessening the severity of, or symptomatically treating cystic fibrosis in a patient, such as a mammal, wherein the patient possesses a CFTR genetic mutation selected from E193K, F1052V and G1069R. In some embodiments, the method produces an increase in chloride transport relative to baseline chloride transport of the patient of the patient.

In some embodiments, disclosed herein is a method of treating, lessening the severity of, or symptomatically treating cystic fibrosis in a patient, such as a mammal, wherein the patient possesses a CFTR genetic mutation selected from R117C, D110H, R347H, R352Q, E56K, P67L, L206W, A455E, D579G, S1235R, S945L, R1070W, F1074L, D110E, D1270N and D1152H. In some embodiments, the method produces an increase in chloride transport above the baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G. In some embodiments, disclosed herein is a method of treating, lessening the severity of, or symptomatically treating cystic fibrosis in a patient, such as a mammal, wherein the patient possesses a CFTR genetic mutation selected from 1717-1G->A, 1811+1.6kbA->G, 2789+5G->A, 3272-26A->G and 3849+10kbC->T. In some embodiments, disclosed herein is a method of treating, lessening the severity of, or symptomatically treating cystic fibrosis in a patient, such as a mammal, wherein the patient possesses a CFTR genetic mutation selected from 2789+5G->A and 3272-26A->G.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V and G1069R, and a human CFTR mutation selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R and S1251N, and a human CFTR mutation selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from E193K, F1052V and G1069R, and a human CFTR mutation selected from F508del, R117H, and G551D. In some embodiments, the method produces an increase in chloride transport relative to baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from R117C, D110H, R347H, R352Q, E56K, P67L, L206W, A455E, D579G, S1235R, S945L, R1070W, F1074L, D110E, D1270N and D1152H, and a human CFTR mutation selected from F508del, R117H, and G551D. In some embodiments, the method produces an increase in chloride transport which is above the baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from 1717-1G->A, 1811+1.6kbA->G, 2789+5G->A, 3272-26A->G and 3849+10kbC->T, and a human CFTR mutation selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from 2789+5G->A and 3272-26A->G, and a human CFTR mutation selected from F508del, R117H.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V and G1069R. In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R and S1251N. In some embodiments, disclosed herein is a method of treating, lessening the severity of, or symptomatically treating cystic fibrosis in a patient, such as a mammal, wherein the patient possesses a CFTR genetic mutation selected from E193K, F1052V and G1069R. In some embodiments, the method produces an increase in chloride transport relative to baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from R117C, D110H, R347H, R352Q, E56K, P67L, L206W, A455E, D579G, S1235R, S945L, R1070W, F1074L, D110E, D1270N and D1152H. In some embodiments, the method produces an increase in chloride transport which is above the baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G. In some embodiments, disclosed herein is a method of treating, lessening the severity of, or symptomatically treating cystic fibrosis in a patient, such as a mammal, wherein the patient possesses a CFTR genetic mutation selected from 1717-1G->A, 1811+1.6kbA->G, 2789+5G->A, 3272-26A->G and 3849+10kbC->T. In some embodiments, disclosed herein is a method of treating, lessening the severity of, or symptomatically treating cystic fibrosis in a patient, such as a mammal, wherein the patient possesses a CFTR genetic mutation selected from 2789+5G->A and 3272-26A->G.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D, and one or more human CFTR mutations selected from F508del, R117H, and G551D.

In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R, S1251N, E193K, F1052V and G1069R, and one or more human CFTR mutations selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from G178R, G551S, G970R, G1244E, S1255P, G1349D, S549N, S549R and S1251N, and one or more human CFTR mutations selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from E193K, F1052V and G1069R, and one or more human CFTR mutations selected from F508del, R117H, and G551D. In some embodiments, the method produces an increase in chloride transport relative to baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from R117C, D110H, R347H, R352Q, E56K, P67L, L206W, A455E, D579G, S1235R, S945L, R1070W, F1074L, D110E, D1270N and D1152H, and one or more human CFTR mutations selected from F508del, R117H, and G551D. In some embodiments, the method produces an increase in chloride transport which is above the baseline chloride transport of the patient.

In some embodiments, disclosed herein is a method of treating, lessening the severity of, or symptomatically treating cystic fibrosis in a patient, such as a mammal, wherein the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and one or more human CFTR mutations selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from 1717-1G->A, 1811+1.6kbA->G, 2789+5G->A, 3272-26A->G and 3849+10kbC->T, and one or more human CFTR mutations selected from F508del, R117H, and G551D. In some embodiments, disclosed herein is a method of treating, lessening the severity of, or symptomatically treating cystic fibrosis in a patient, such as a mammal, wherein the patient possesses a CFTR genetic mutation selected from 2789+5G->A and 3272-26A->G, and one or more human CFTR mutations selected from F508del, R117H, and G551D.

In some embodiments, the patient is heterozygous having one CF-causing mutation on one allele and another CF-causing mutation on the other allele. In some embodiments, the patient is heterozygous for F508del, and the other CFTR genetic mutation is any CF-causing mutation, including, but not limited to F508del on one CFTR allele and a CFTR mutation on the second CFTR allele that is associated with minimal CFTR function, residual CFTR function, or a defect in CFTR channel gating activity.

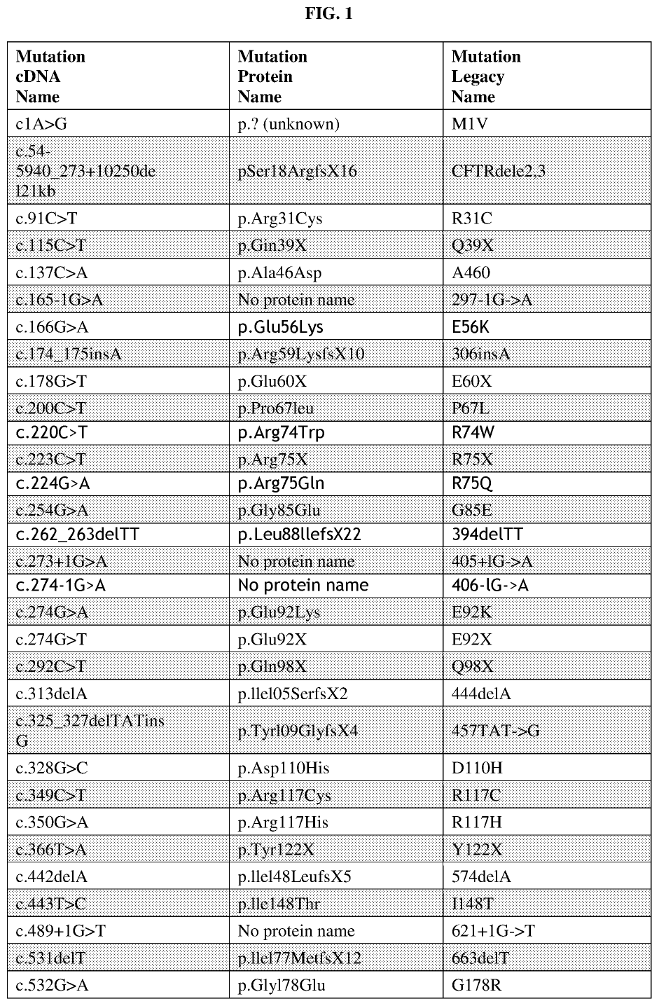

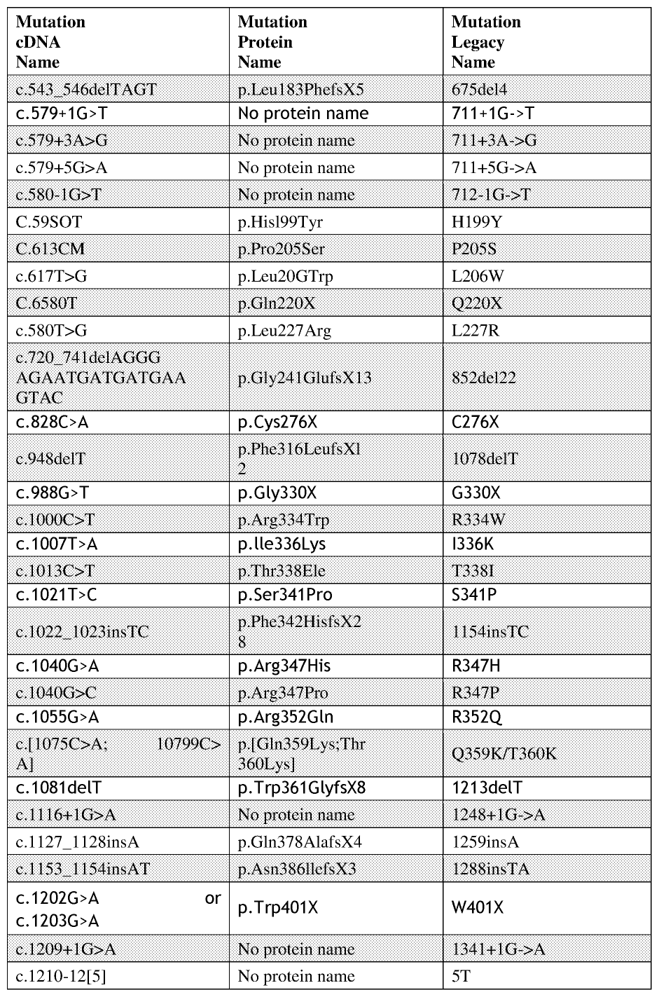

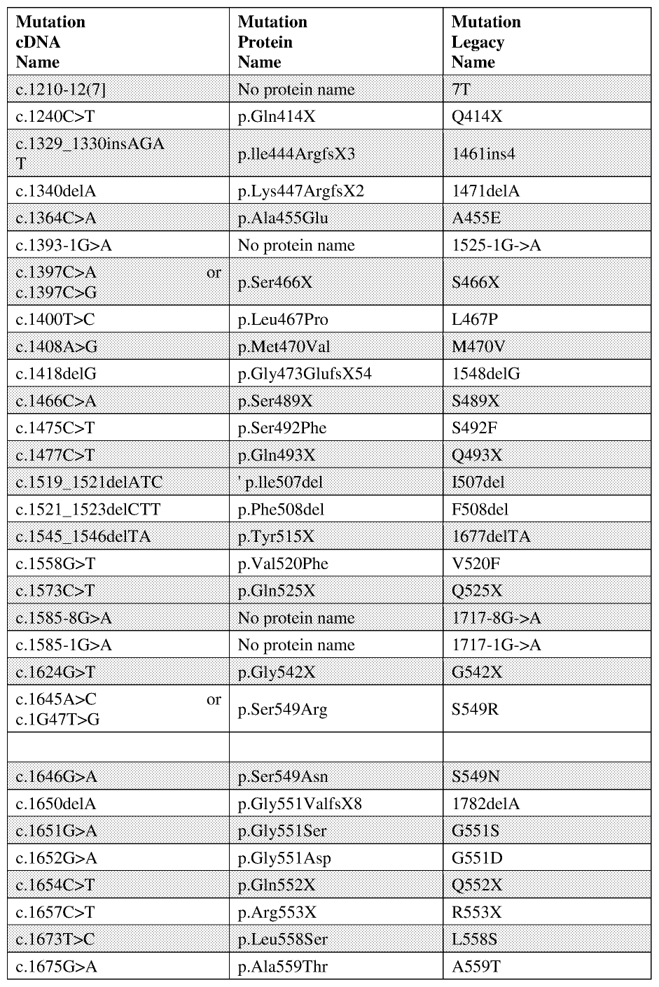

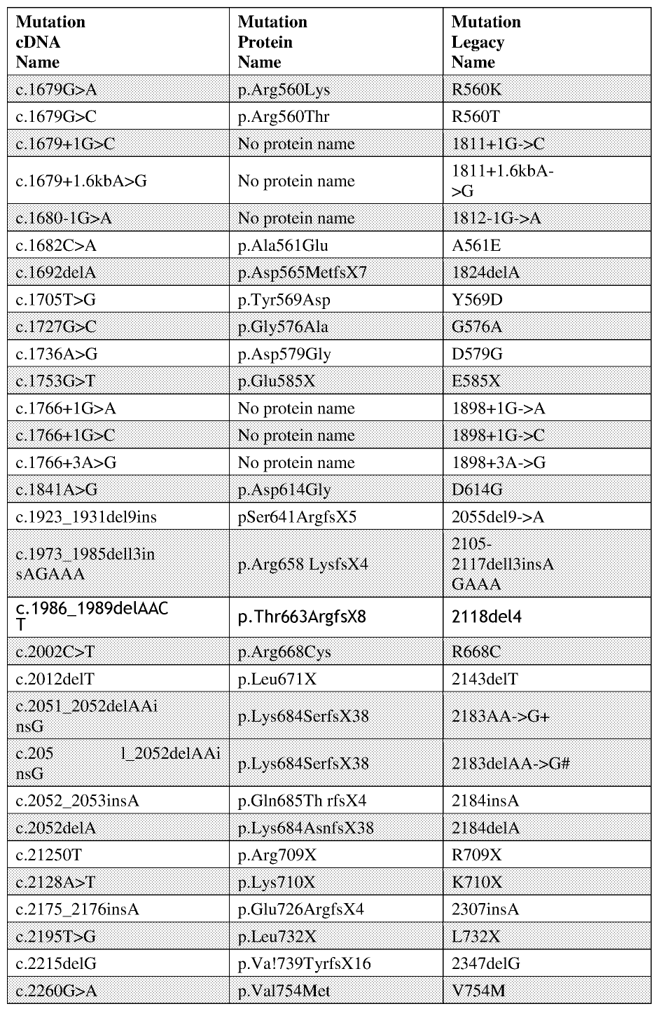

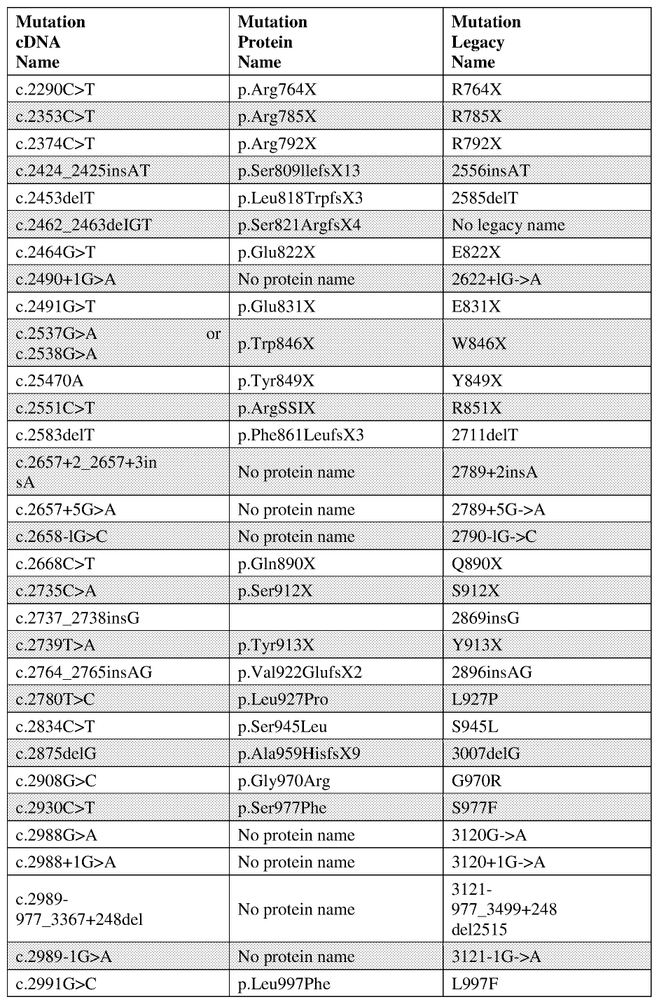

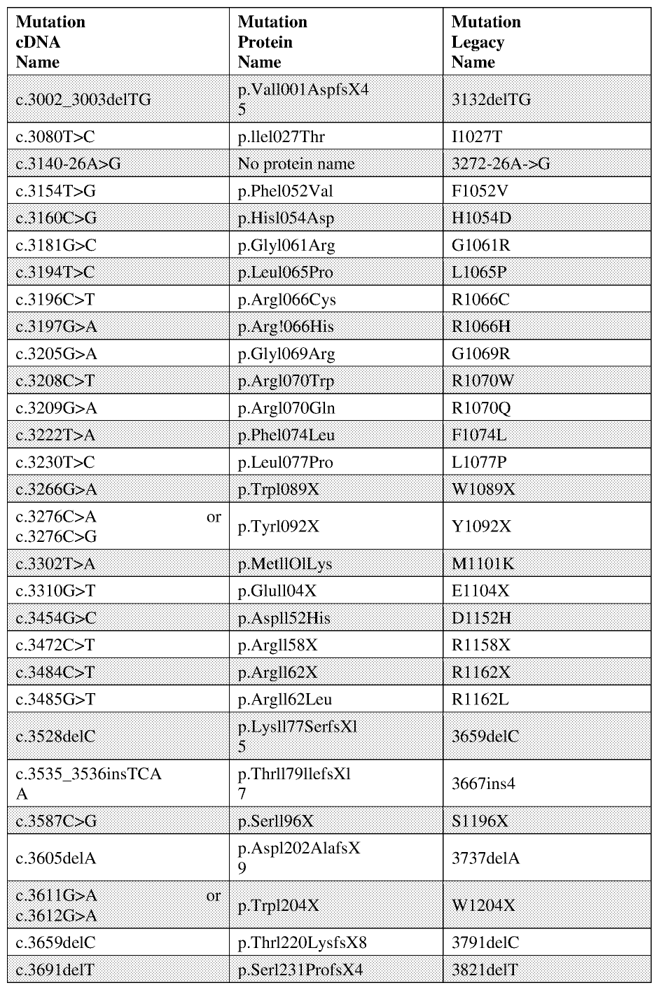

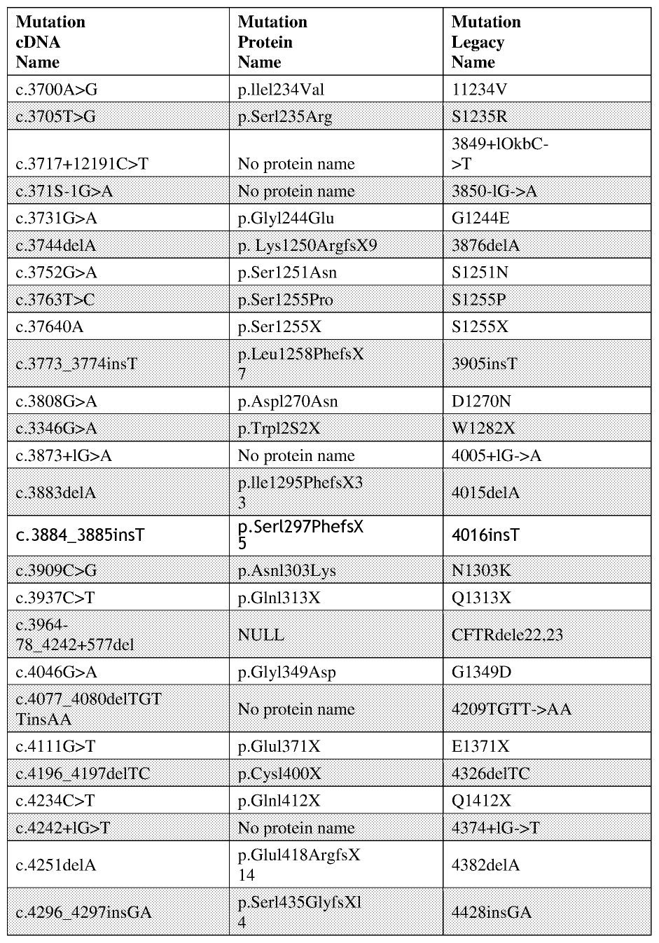

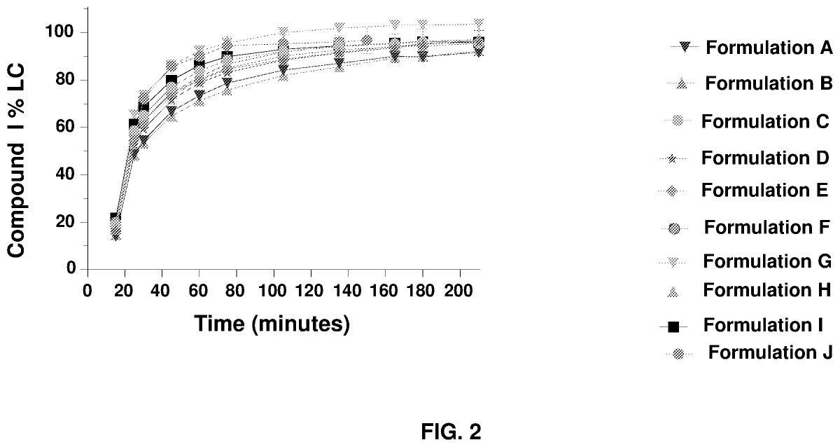

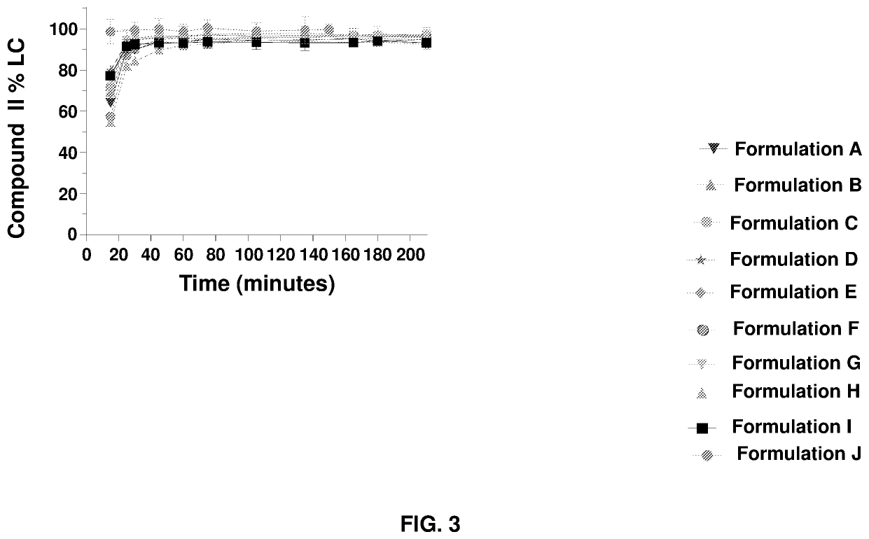

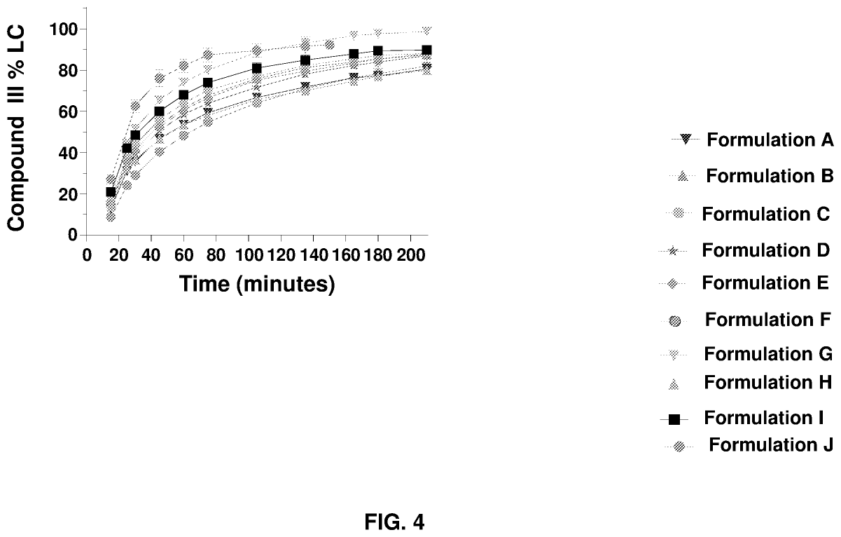

In some embodiments, the CF-causing mutation is selected from Table 5. In some embodiments, the patient is heterozygous having one CF-causing mutation on one CFTR allele selected from the mutations listed in the table from FIG. 1 and another CF-causing mutation on the other CFTR allele is selected from the CFTR mutations listed in Table 5.

TABLE 5
CFTR Mutations

Q39X
W57X
E60X
R75X
E92X
Q98X
Y122X
L218X
Q220X
C276X
Q290X
G330X
W401X
Q414X
S434X
S466X
S489X
Q493X
W496X
Q525X
G542X
Q552X
R553X
E585X
G673X
R709X
K710X
L732X
R764X
R785X
R792X
E822X
W846X
R851X
Q890X
S912X
W1089X
Y1092X
E1104X
R1158X
R1162X
S1196X
W1204X
S1255X
W1282X
Q1313X
621+1G→T
711+1G→T
711+5G→A
712−1G→T
405+1G→A
405+3A→C
406−1G→A
621+1G→T
1248+1G→A
1341+1G→A
1717−1G→A
1811+1.6kbA→G
1811+1G→C
1812−1G→A
1898+1G→A
2622+1G→A
3120+1G→A
3120G→A
3850−1G→A
4005+1G→A
4374+1G→T
663delT
2183AA→G
CFTRdel2,3
3659delC
394delTT
2184insA
3905insT
2184delA
1078delT
1154insTC
2183delAA→G
2143delT
1677delTA
3876delA
2307insA
4382delA
4016insT
2347delG
3007delG
574delA
2711delT
3791delC
CFTRdele22-23
457TAT→G
2043delG
2869insG
3600+2insT
3737delA
4040delA
541delC
A46D
T338I
R347P
L927P
G85E
S341P
L467P
I507del
V520F
A559T
R560T
R560S
A561E
Y569D
L1065P
R1066C
R1066M
L1077P
H1085R
M1101K
N1303K
2789+5G→A
3849+10kbC→T
3272−26A→G
711+3A→G
E56K
P67L
R74W
D110E
D110H
R117C
L206W
R347H
R352Q
A455E
D579G
E831X
S945L
S977F
F1052V
R1070W
F1074L
D1152H
D1270N
R117H
G178R
S549N
S549R
G551D
G551S
G1244E
S1251N
S1255P
G1349D

TABLE 6
CFTR Mutations

| Criteria | Mutation | | | | | | |
|---|---|---|---|---|---|---|---|
| Truncation | S4X | C276X | | G542X | | R792X | E1104X |
| mutations | G27X | Q290X | | G550X | | E822X | R1158X |
| % PI >50% | Q39X | G330X | | Q552X | | W846X | R1162X |
| and/or | W57X | W401X | | R553X | | Y849X | S1196X |
| SwCl⁻ >86 | E60X | Q414X | | E585X | | R851X | W1204X |
| mmol/L | R75X | S434X | | G673X | | Q890X | L1254X |
| no full-length | E92X | S466X | | Q685X | | S912X | S1255X |
| protein | Q98X | S489X | | R709X | | Y913X | W1282X |
| | Y122X | Q493X | | K710X | | W1089X | Q1313X |
| | E193X | W496X | | L732X | | Y1092X | E1371X |
| | L218X | C524X | | R764X | | W1098X | Q1382X |
| | Q220X | Q525X | | R785X | | R1102X | Q1411X |
| Splice mutations | 185+1G→T | 711+5G→A | | 1717−8G→A | | 2622+1G→A | 3121−1G→A |
| % PI >50% | 296+1G→A | 712−1G→T | | 1717−1G→A | | 2790−1G→C | 3500−2A→G |
| and/or | 405+1G→A | 1248+1G→A | | 1811+1G→C | | 3040G→C | 3600+2insT |
| SwCl⁻ >86 | | | | | | (G970R) | |
| mmol/L | 405+3A→C | 1249−1G→A | | 1811+1.6kbA→G | | | 3850−1G→A |
| no or little | 406−1G→A | 1341+1G→A | | 1812−1G→A | | 3120G→A | 4005+1G→A |
| mature mRNA | 621+1G→T | 1525−2A→G | | 1898+1G→A | | 3120+1G→A | 4374+1G→T |
| | 711+1G→T | 1525−1G→A | | 1898+1G→C | | 3121−2A→G | |
| Small (≤3 | 182delT | 1119delA | | 1782delA | | 2732insA | 3876delA |
| nucleotide) | 306insA | 1138insG | | 1824delA | | 2869insG | 3878delG |
| insertion/deletion | 365-366insT | 1154insTC | | 2043delG | | 2896insAG | 3905insT |
| (ins/del) | 394delTT | 1161delC | | 2143delT | | 2942insT | 4016insT |
| frameshift | 442delA | 1213delT | | 2183AA→G ^{a} | | 2957delT | 4021dupT |
| mutations | 444delA | 1259insA | | 2184delA | | 3007delG | 4040delA |
| % PI >50% | 457TAT→G | 1288insTA | | 2184insA | | 3028delA | 4279insA |
| and/or | 541delC | 1471delA | | 2307insA | | 3171delC | 4326delTC |
| SwCl⁻ >86 | 574delA | 1497delGG | | 2347delG | | 3659delC | |
| mmol/L | 663delT | 1548delG | | 2585delT | | 3737delA | |
| garbled and/or | 935delA | 1609del CA | | 2594delGT | | 3791delC | |
| truncated | 1078delT | 1677delTA | | 2711delT | | 3821delT | |
| protein | | | | | | | |
| Non-small (>3 | CFTRdele2,3 | | 1461ins4 | | 2991del32 | | |
| nucleotide) | CFTRdele22,23 | | 1924del7 | | 3667ins4 | | |
| insertion/deletion | 124del23bp | | 2055del9→A | | 4010del4 | | |
| (ins/del) | 852del22 | | 2105-2117del13insAGAAA | | 4209TGTT→AA | | |
| frameshift | 991del5 | | 2721del11 | | | | |
| mutations | | | | | | | |
| % PI >50% | | | | | | | |
| and/or | | | | | | | |
| SwCl⁻ >86 | | | | | | | |
| mmol/L | | | | | | | |
| garbled | | | | | | | |
| and/or | | | | | | | |
| truncated | | | | | | | |
| protein | | | | | | | |
| Class II, III, IV | A46D^{b} | V520F | | Y569D^{b} | N1303K | | |
| mutations not | G85E | A559T^{b} | | L1065P | | | |
| responsive to | R347P | R560T | | R1066C | | | |
| Compound III | L467P^{b} | R560S | | L1077P^{b} | | | |
| alone or in | I507del | A561E | | M1101K | | | |
| combination with | | | | | | | |
| Compound II or | | | | | | | |
| Compound IV | | | | | | | |
| % PI > 50% | | | | | | | |
| and/or | | | | | | | |
| SwCl >86 | | | | | | | |
| mmol/L AND | | | | | | | |
| Not responsive | | | | | | | |
| in vitro to | | | | | | | |
| Compound III | | | | | | | |
| alone or in | | | | | | | |
| combination with | | | | | | | |
| Compound II or | | | | | | | |
| Compound IV | | | | | | | |

Note:
% PI: percentage of F508del-CFTR heterozygous patients in the CFTR2 patient registry who are pancreatic insufficient; SwCl⁻: mean sweat chloride of F508del-CFTR heterozygous patients in the CFTR2 patient registry
^{a} Also known as 2183delAA→G.
^{b}Unpublished data.

Table 6 above includes certain exemplary CFTR minimal function mutations, which are detectable by an FDA-cleared genotyping assay, but does not include an exhaustive list.

In some embodiments, disclosed herein is a method of treating, lessening the severity of, or symptomatically treating cystic fibrosis in a patient with F508del/MF (F/MF) genotypes (heterozygous for F508del and an MF mutation not expected to respond to CFTR modulators, such as Compound III); with F508del/F508del (F/F) genotype (homozygous for F508del); and/or with F508del/gating (F/G) genotypes (heterozygous for F508del and a gating mutation known to be CFTR modulator-responsive (e.g., Compound III-responsive). In some embodiments, a patient with F508del/MF (F/MF) genotypes has a MF mutation that is not expected to respond to Compound II, Compound III, and both of Compound II and Compound III. In some embodiments, a patient with F508del/MF (F/MF) genotypes has any one of the MF mutations in Table 6.

In some embodiments, the patient is heterozygous for F508del, and the other CFTR genetic mutation is any CF-causing mutation, including truncation mutations, splice mutations, small (≤3 nucleotide) insertion or deletion (ins/del) frameshift mutations; non-small (>3 nucleotide) insertion or deletion (ins/del) frameshift mutations; and Class II, III, IV mutations not responsive to Compound III alone or in combination with Compound II or Compound IV.

In some embodiments, the patient is heterozygous for F508del, and the other CFTR genetic mutation is a truncation mutation. In some specific embodiments, the truncation mutation is a truncation mutation listed in Table 6.

In some embodiments, the patient is heterozygous for F508del, and the other CFTR genetic mutation is a splice mutation. In some specific embodiments, the splice mutation is a splice mutation listed in Table 6.

In some embodiments, the patient is heterozygous for F508del, and the other CFTR genetic mutation is a small (≤3 nucleotide) insertion or deletion (ins/del) frameshift mutation. In some specific embodiments, the small (≤3 nucleotide) insertion or deletion (ins/del) frameshift mutation is a small (≤3 nucleotide) insertion or deletion (ins/del) frameshift mutation listed in Table 6.

In some embodiments, the patient is heterozygous for F508del, and the other CFTR genetic mutation is any CF-causing mutation expected to be and/or is responsive to, based on in vitro and/or clinical data, the combination of Compound I and pharmaceutically acceptable salts thereof, Compound II and pharmaceutically acceptable salts thereof, and Compound III and pharmaceutically acceptable salts thereof.

In some embodiments, the patient is heterozygous for F508del, and the other CFTR genetic mutation is any CF-causing mutation expected to be and/or is responsive, based on in vitro and/or clinical data, to the triple combination of Compound I and pharmaceutically acceptable salts thereof, Compound II and pharmaceutically acceptable salts thereof, and Compound III and pharmaceutically acceptable salts thereof.

In some embodiments, the patient is heterozygous for F508del, and the other CFTR genetic mutation is a non-small (>3 nucleotide) insertion or deletion (ins/del) frameshift mutation. In some specific embodiments, the non-small (>3 nucleotide) insertion or deletion (ins/del) frameshift mutation is a non-small (>3 nucleotide) insertion or deletion (ins/del) frameshift mutation listed in Table 6.

In some embodiments, the patient is heterozygous for F508del, and the other CFTR genetic mutation is a Class II, III, IV mutations not responsive to Compound III alone or in combination with Compound II. In some specific embodiments, the Class II, III, IV mutations not responsive to Compound III alone or in combination with Compound II is a Class II, III, IV mutations not responsive to Compound III alone or in combination with Compound II or Compound IV listed in Table 6.

In some embodiments, the patient is heterozygous for F508del, and the other CFTR genetic mutation is any mutation listed in Table 6.

In some embodiments, the patient is heterozygous for F508del, and the other CFTR genetic mutation is any mutation listed in Table 5, 6, and FIG. 1.

In some embodiments, the patient is heterozygous for F508del, and the other CFTR genetic mutation is any mutation listed in Table 5. In some embodiments, the patient is heterozygous for F508del, and the other CFTR genetic mutation is any mutation listed in Table 5. In some embodiments, the patient is heterozygous for F508del, and the other CFTR genetic mutation is any mutation listed in FIG. 1.

In some embodiments, the patient is homozygous for F508del.

In some embodiments, the patient is heterozygous having one CF-causing mutation on one CFTR allele selected from the mutations listed in the table from FIG. 1 and another CF-causing mutation on the other CFTR allele is selected from the CFTR mutations listed in Table 6.

Patients with an F508del/gating mutation genotype are defined as patients that are heterozygous F508del-CFTR with a second CFTR allele that contains a mutation associated with a gating defect and clinically demonstrated to be responsive to Compound III. Examples of such mutations include: G178R, S549N, S549R, G551D, G551S, G1244E, S1251N, 51255P, and G1349D.

Patients with an F508del/residual function genotype are defined as patients that are heterozygous F508del-CFTR with a second CFTR allele that contains a mutation that results in reduced protein quantity or function at the cell surface which can produce partial CFTR activity. CFTR gene mutations known to result in a residual function phenotype include in some embodiments, a CFTR residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, and K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, A1067T, E193K, or K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, or A1067T.

In some embodiments, disclosed herein is a method of treating, lessening the severity of, or symptomatically treating cystic fibrosis in a patient, such as a mammal, wherein the patient possesses a CFTR genetic mutation selected from the mutations listed in FIG. 1.

In some embodiments, the tablets disclosed herein is useful for treating, lessening the severity of, or symptomatically treating cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl⁻ concentrations, or ex vivo biochemical or histochemical techniques to monitor cell surface density. Using such methods, residual CFTR activity can be readily detected for patients that are heterozygous or homozygous for a variety of different mutations, including patients heterozygous for the most common mutation, F508del, as well as other mutations such as the G551D mutation, or the R117H mutation. In some embodiments, tablets disclosed herein are useful for treating, lessening the severity of, or symptomatically treating cystic fibrosis in patients who exhibit little to no residual CFTR activity. In some embodiments, the tablets disclosed herein are useful for treating, lessening the severity of, or symptomatically treating cystic fibrosis in patients who exhibit little to no residual CFTR activity in the apical membrane of respiratory epithelia.

In some embodiments, the tablets disclosed herein are useful for treating or lessening the severity of cystic fibrosis in patients who exhibit residual CFTR activity using pharmacological methods. Such methods increase the amount of CFTR present at the cell surface, thereby inducing a hitherto absent CFTR activity in a patient or augmenting the existing level of residual CFTR activity in a patient.

In some embodiments, the tablets disclosed herein are useful for treating or lessening the severity of cystic fibrosis in patients with certain genotypes exhibiting residual CFTR activity.

In some embodiments, the tablets disclosed herein are useful for treating, lessening the severity of, or symptomatically treating cystic fibrosis in patients within certain clinical phenotypes, e.g., a mild to moderate clinical phenotype that typically correlates with the amount of residual CFTR activity in the apical membrane of epithelia. Such phenotypes include patients exhibiting pancreatic sufficiency.

In some embodiments, the tablets disclosed herein are useful for treating, lessening the severity of, or symptomatically treating patients diagnosed with pancreatic sufficiency, idiopathic pancreatitis and congenital bilateral absence of the vas deferens, or mild lung disease wherein the patient exhibits residual CFTR activity.

In some embodiments, this disclosure relates to a method of augmenting or inducing anion channel activity in vitro or in vivo, comprising contacting the channel with a tablet disclosed herein. In some embodiments, the anion channel is a chloride channel or a bicarbonate channel. In some embodiments, the anion channel is a chloride channel.

The exact amount of API(s) and tablets comprising such API(s) required will vary from subject to subject, depending on the species, age, and general condition of the subject, the severity of the disease, the particular agent, its mode of administration, and the like. The compounds of this disclosure may be formulated in dosage unit form for ease of administration and uniformity of dosage. The expression “dosage unit form” as used herein refers to a physically discrete unit of agent appropriate for the patient to be treated. It will be understood, however, that the total daily usage of API(s) and tablets comprising such API(s) of this disclosure will be decided by the attending physician within the scope of sound medical judgment. The specific effective dose level for any particular patient or organism will depend upon a variety of factors including the disorder being treated and the severity of the disorder; the activity of the specific API employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, such as a mammal, and even further such as a human.

In some embodiments, the disclosure also is directed to methods of treatment using isotope-labelled compounds of the afore-mentioned compounds, which have the same structures as disclosed herein except that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “²H” or “D.”

The deuterium (²H)-labelled compounds and salts can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417; and T. G. Gant “Using deuterium in drug discovery: leaving the label in the drug” J. Med. Chem. 2014, 57, 3595-3611, relevant portions of which are independently incorporated herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

One of ordinary skill in the art would understand that deuteration of one or more metabolically labile positions on a compound or active metabolite may lead to improvement of one or more superior DMPK properties while maintaining biological activity as compared to the corresponding hydrogen analogs. The superior DMPK property or properties may have an impact on the exposure, half-life, clearance, metabolism, and/or even food requirements for optimal absorption of the drug product. Deuteration may also change the metabolism at other non-deuterated positions of the deuterated compound.

In some embodiments, Compound III′ as used herein includes the deuterated compound disclosed in U.S. Pat. No. 8,865,902 (which is incorporated herein by reference), and CTP-656.

In some embodiments, Compound III′ is:

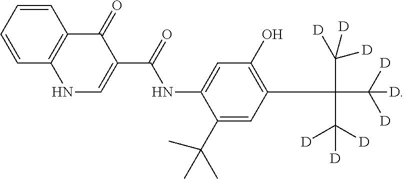

Exemplary embodiments of the disclosure include:

1. A single tablet comprising a first solid dispersion, a second solid dispersion, and a third solid dispersion,

(a) wherein the first solid dispersion comprises 50 mg to 300 mg of Compound I:

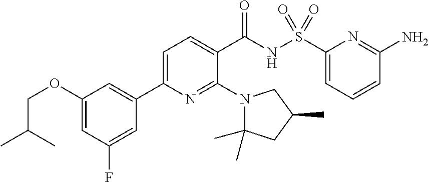 [SMILES: CC(C)COc1cc(F)cc(-c2ccc(C(=O)NS(=O)(=O)c3cccc(N)n3)c(N3C[C@@H](C)CC3(C)C)n2)c1]

and 10 wt % to 60 wt % of a polymer relative to the total weight of the first solid dispersion;

(b) wherein the second solid dispersion comprises 10 mg to 50 mg of Compound II:

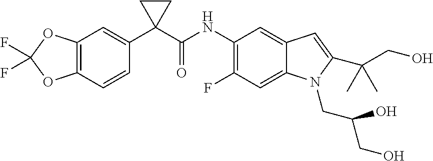 [SMILES: CC(C)(CO)c1cc2cc(NC(=O)C3(c4ccc5c(c4)OC(F)(F)O5)CC3)c(F)cc2n1C[C@@H](O)CO]

and 10 wt % to 30 wt % of a polymer relative to the total weight of the second solid dispersion; and

(c) wherein the third solid dispersion comprises 25 mg to 200 mg of Compound III:

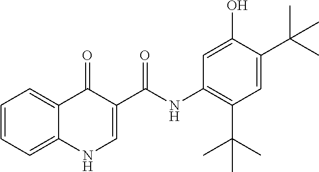 [SMILES: CC(C)(C)c1cc(C(C)(C)C)c(NC(=O)c2c[nH]c3ccccc3c2=O)cc1O]

and 10 wt % to 30 wt % of a polymer relative to the total weight of the third solid dispersion.
2. The single tablet of embodiment 1, wherein the polymer in the first solid dispersion is present in 10 wt % to 50 wt %, 10 wt % to 40 wt %, or 10 wt % to 30 wt %, relative to the total weight of the first solid dispersion.
3. The single tablet of embodiment 1, wherein the polymer in the first solid dispersion is present in 15 wt % to 25 wt % relative to the total weight of the first solid dispersion.
4. The single tablet of embodiment 1, wherein the polymer in the first solid dispersion is present in 20 wt % relative to the total weight of the first solid dispersion.
5. The single tablet of any one of embodiments 1-4, wherein at least one of the first, second, and third solid dispersions is a spray-dried dispersion.
6. The single tablet of any one of embodiments 1-4, wherein each of the first, second, and third solid dispersions is a spray-dried dispersion.
7. The single tablet of any one of embodiments 1-6, wherein each of said polymers in the first solid dispersion, second solid dispersion, and third solid dispersion comprises one or more polymers independently chosen from cellulose-based polymers, polyoxyethylene-based polymers, polyethylene-propylene glycol copolymers, vinyl-based polymers, PEO-polyvinyl caprolactam-based polymers, and polymethacrylate-based polymers.
8. The single tablet of embodiment 7:
wherein the cellulose-based polymer is chosen from a methylcellulose, a hydroxypropyl methylcellulose (hypromellose), a hypromellose phthalate (HPMC-P), and a hypromellose acetate succinate;
wherein the polyoxyethylene-based polymer or polyethylene-propylene glycol copolymer is chosen from a polyethylene glycol and a poloxamer;
wherein the vinyl-based polymer is a polyvinylpyrrolidine;
wherein the PEO-polyvinyl caprolactam-based polymer is a polyethylene glycol, polyvinyl acetate and polyvinylcaprolactam-based graft copolymer; and
wherein the polymethacrylate-based polymer is a poly(methacrylic acid, ethyl acrylate) (1:1) or a dimethylaminoethyl methacrylate-methylmethacrylate copolymer.
9. The single tablet of embodiment 8, wherein the cellulose-based polymer is a hypromellose acetate succinate and a hypromellose, or a combination of hypromellose acetate succinate and a hypromellose.
10. The single tablet of embodiment 9, wherein the cellulose-based polymer is chosen from hypromellose E15, hypromellose acetate succinate L, and hypromellose acetate succinate H.
11. The single tablet of embodiment 9, wherein the polyoxyethylene-based polymer or polyethylene-propylene glycol copolymer is chosen from polyethylene glycol 3350 and poloxamer 407.
12. The single tablet of embodiment 9, wherein the vinyl-based polymer is chosen from polyvinylpyrrolidine K30 and polyvinylpyrrolidine VA 64.
13. The single tablet of embodiment 9, wherein the polymethacrylate polymer is chosen from Eudragit L100-55 and Eudragit E PO.
14. The single tablet of embodiment 7, wherein said polymer for the first solid dispersion is chosen from a hypromellose acetate succinate and a hypromellose, and a combination thereof; said polymer for the second solid dispersion is a hypromellose; and said polymer for the third solid dispersion is a hypromellose acetate succinate.
15. The single tablet of embodiment 7, wherein said polymer for the first solid dispersion is a hypromellose acetate succinate; said polymer for the second solid dispersion is hypromellose; and said polymer for the third solid dispersion is a hypromellose acetate succinate.
16. The single tablet of embodiment 7, wherein said polymer for the first solid dispersion is chosen from hydroxypropyl methylcellulose (HPMC) E15, hypromellose acetate succinate L, hypromellose acetate succinate H, and a combination thereof; said polymer for the second solid dispersion is HPMC E15; and said polymer for the third solid dispersion is hypromellose acetate succinate H.
17. The single tablet of embodiment 7, wherein said polymer for the first solid dispersion is hypromellose acetate succinate H; said polymer for the second solid dispersion is HPMC E15; and said polymer for the third solid dispersion is hypromellose acetate succinate H.
18. The single tablet of embodiment 7, wherein said polymer for the first solid dispersion is hypromellose acetate succinate HG; said polymer for the second solid dispersion is HPMC E15; and said polymer for the third solid dispersion is hypromellose acetate succinate HG.
19. The single tablet of any one of embodiments 1-18, wherein the first solid dispersion comprises 50 mg to 600 mg of Compound I.
20. The single tablet of any one of embodiments 1-18, wherein the first solid dispersion comprises 50 mg to 200 mg, 75 mg to 200 mg, 50 mg, 75 mg, 100 mg, 150 mg, 200 mg, or 300 mg of Compound I.
21. The single tablet of any one of embodiments 1-18, wherein the first solid dispersion comprises 100 mg of Compound I.
22. The single tablet of any one of embodiments 1-18, wherein the first solid dispersion comprises 150 mg of Compound I.
23. The single tablet of any one of embodiments 1-22, wherein the second solid dispersion comprises 15 mg to 50 mg of Compound II.
24. The single tablet of any one of embodiments 1-22, wherein the second solid dispersion comprises 20 mg to 35 mg of Compound II.
25. The single tablet of any one of embodiments 1-22, wherein the second solid dispersion comprises 10 mg to 30 mg, 15 mg to 30 mg, or 20 mg to 30 mg of Compound II.
26. The single tablet of any one of embodiments 1-22, wherein the third solid dispersion comprises 50 mg to 200 mg of Compound III.
27. The single tablet of any one of embodiments 1-22, wherein the third solid dispersion comprises 50 mg to 175 mg, 50 mg to 100 mg, or 50 mg to 80 mg of Compound III.
28. The single tablet of any one of embodiments 1-18, wherein:
the first solid dispersion comprises 50 mg to 200 mg of Compound I:
the second solid dispersion comprises 15 mg to 50 mg of Compound II: and
the third solid dispersion comprises 50 mg to 200 mg of Compound.
29. The single tablet of any one of embodiments 1-18, wherein:
the first solid dispersion comprises 75 mg to 200 mg of Compound I:
the second solid dispersion comprises 10 mg to 30 mg of Compound II: and
the third solid dispersion comprises 50 mg to 100 mg of Compound.
30. The single tablet of any one of embodiments 1-18, wherein:
the first solid dispersion comprises 100 mg to 200 mg of Compound I:
the second solid dispersion comprises 20 mg to 30 mg of Compound II: and
the third solid dispersion comprises 50 mg to 80 mg of Compound III.
31. The single tablet of any one of embodiments 1-18, wherein Compounds I, II, and III are in a weight ratio of Compound I:Compound II:Compound III 4 to 6:1:3 to 5.
32. The single tablet of any one of embodiments 1-18, wherein Compounds I, II, and III are in a weight ratio of Compound I:Compound II:Compound III 4 to 6:1:3.
33. The single tablet of any one of embodiments 1-32, comprising one or more excipients chosen from a filler, a disintegrant, a surfactant, and a lubricant.
34. The single tablet of embodiment 33, wherein the filler is chosen from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.
35. The single tablet of embodiment 33, wherein the disintegrant is chosen from croscarmellose sodium, sodium starch glycolate, crospovidone, corn or pre-gelatinized starch, sodium carboxymethyl cellulose, calcium carboxymethyl cellulose, and microcrystalline cellulose.
36. The single tablet of embodiment 33, wherein the lubricant is chosen from magnesium stearate, sodium stearyl fumarate, calcium stearate, sodium stearate, stearic acid, and talc; and wherein the surfactant is chosen from sodium lauryl sulfate, poloxamers, docusate sodium, PEGs and PEG derivatives.
37. The single tablet of any one of embodiments 1-36, wherein each of Compounds I, II and III is independently substantially amorphous.
38. A single tablet comprising:
(a) 20 wt % to 50 wt % of a first solid dispersion relative to the total weight of the tablet;
(b) 10 wt % to 30 wt % of a second solid dispersion relative to the total weight of the tablet; and
(c) 3 wt % to 10 wt % of a third solid dispersion relative to the total weight of the tablet;

wherein the first solid dispersion comprises 40 wt % to 90 wt % of Compound I:

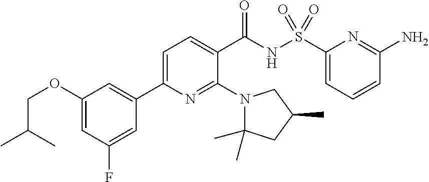 [SMILES: CC(C)COc1cc(F)cc(-c2ccc(C(=O)NS(=O)(=O)c3cccc(N)n3)c(N3C[C@@H](C)CC3(C)C)n2)c1]

and 10 wt % to 60 wt % of a polymer relative to the total weight of the first solid dispersion;

wherein the second solid dispersion comprises 70 wt % to 90 wt % of Compound II:

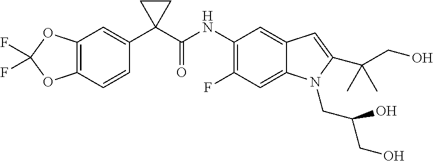 [SMILES: CC(C)(CO)c1cc2cc(NC(=O)C3(c4ccc5c(c4)OC(F)(F)O5)CC3)c(F)cc2n1C[C@@H](O)CO]

and 10 wt % to 30 wt % of a polymer relative to the total weight of the second solid dispersion; and
wherein the third solid dispersion comprises 70 wt % to 90 wt % of Compound III:

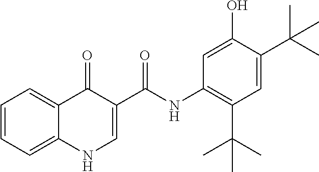 [SMILES: CC(C)(C)c1cc(C(C)(C)C)c(NC(=O)c2c[nH]c3ccccc3c2=O)cc1O]

and 10 wt % to 30 wt % of a polymer relative to the total weight of the third solid dispersion.
39. The single tablet of embodiment 38, wherein the polymer in the first solid dispersion is present in 10 wt % to 50 wt %, 10 wt % to 40 wt %, or 10 wt % to 30 wt % relative to the total weight of the first solid dispersion.
40. The single tablet of embodiment 38, wherein the polymer in the first solid dispersion is present in 15 wt % to 25 wt % relative to the total weight of the first solid dispersion.
41. The single tablet of embodiment 38, wherein the polymer in the first solid dispersion is present in 20 wt % relative to the total weight of the first solid dispersion.
42. The single tablet of any one of embodiments 38-41, wherein at least one of the first, second, and third solid dispersions is a spray-dried dispersion.
43. The single tablet of any one of embodiments 38-41, wherein each of the first, second, and third solid dispersions is a spray-dried dispersion.
44. The single tablet of any one of embodiments 38-43, wherein each of said polymers in the first solid dispersion, second solid dispersion, and third solid dispersion comprises one or more polymers independently chosen from cellulose-based polymers, polyoxyethylene-based polymers, polyethylene-propylene glycol copolymers, vinyl-based polymers, PEO-polyvinyl caprolactam-based polymers, and polymethacrylate-based polymers.
45. The single tablet of embodiment 44,
wherein the cellulose-based polymer is chosen from a
methylcellulose, a hydroxypropyl methylcellulose (hypromellose), a hypromellose phthalate (HPMC-P), and a hypromellose acetate succinate;
wherein the polyoxyethylene-based polymer or polyethylene-propylene glycol copolymer is chosen from a polyethylene glycol and a poloxamer;
wherein the vinyl-based polymer is a polyvinylpyrrolidine;
wherein the PEO-polyvinyl caprolactam-based polymer is a polyethylene glycol, polyvinyl acetate and polyvinylcaprolactam-based graft copolymer; and
wherein the polymethacrylate-based polymer is a poly(methacrylic acid, ethyl acrylate) (1:1) or a dimethylaminoethyl methacrylate-methylmethacrylate copolymer.
46. The single tablet of embodiment 45, wherein the cellulose-based polymer is a hypromellose acetate succinate and a hypromellose, or a combinations of hypromellose acetate succinate and a hypromellose.
47. The single tablet of embodiment 45, wherein the cellulose-based polymer is chosen from hypromellose E15, hypromellose acetate succinate L and hypromellose acetate succinate H.
48. The single tablet of embodiment 45, wherein the polyoxyethylene-based polymer or polyethylene-propylene glycol copolymer is chosen from polyethylene glycol 3350 and poloxamer 407.
49. The single tablet of embodiment 44, wherein the vinyl-based polymer is chosen from polyvinylpyrrolidine K30 and polyvinylpyrrolidine VA 64.
50. The single tablet of embodiment 44, wherein the polymethacrylate polymer is chosen from Eudragit L100-55 and Eudragit E PO.
51. The single tablet of embodiment 44, wherein said polymer for the first solid dispersion is chosen from a hypromellose acetate succinate and a hypromellose, and a combination thereof; said polymer for the second solid dispersion is a hypromellose; and said polymer for the third solid dispersion is a hypromellose acetate succinate.
52. The single tablet of embodiment 44, wherein said polymer for the first solid dispersion is a hypromellose acetate succinate; said polymer for the second solid dispersion is hypromellose; and said polymer for the third solid dispersion is a hypromellose acetate succinate.
53. The single tablet of embodiment 44, wherein said polymer for the first solid dispersion is chosen from hydroxypropyl methylcellulose E15, hypromellose acetate succinate L, hypromellose acetate succinate H, and a combination thereof; said polymer for the second solid dispersion is hypromellose (HPMC E15); and said polymer for the third solid dispersion is hypromellose acetate succinate H.
54. The single tablet of embodiment 38, wherein:
the second solid dispersion comprises 70 wt % to 85 wt % of Compound II relative to the total weight of the second solid dispersion, and the polymer is hydroxypropyl methylcellulose in an amount of 15 wt % to 30 wt % relative to the total weight of the second solid dispersion; and
the third solid dispersion comprises 70 wt % to 85 wt % of Compound III relative to the total weight of the third solid dispersion, and the polymer is hypromellose acetate succinate in an amount of 15 wt % to 30 wt % relative to the total weight of the second solid dispersion.
55. The single tablet of embodiment 38, wherein:

the second solid dispersion comprises 70 wt % to 85 wt % of Compound II relative to the total weight of the second solid dispersion, and the polymer is hydroxypropyl methylcellulose in an amount of 15 wt % to 30 wt % relative to the total weight of the second solid dispersion; and

the third solid dispersion comprises 80 wt % of Compound III relative to the total weight of the third solid dispersion, and the polymer is hypromellose acetate succinate in an amount of 15 wt % to 20 wt % relative to the total weight of the second solid dispersion.
56. The single tablet of any one of embodiments 38-55, wherein the first solid dispersion comprises 50 wt % to 90 wt % of Compound I.
57. The single tablet of any one of embodiments 38-55, wherein the first solid dispersion comprises 60 wt % to 90 wt % of Compound I.
58. The single tablet of any one of embodiments 38-55, wherein the first solid dispersion comprises 70 wt % to 90 wt % of Compound I.
59. The single tablet of any one of embodiments 38-55, wherein the first solid dispersion comprises 75 wt % to 85 wt % of Compound I.
60. The single tablet of any one of embodiments 38-55, wherein the first solid dispersion comprises 80 wt % of Compound I.
61. The single tablet of any one of embodiments 38-60, wherein the second solid dispersion comprises 75 wt % to 85 wt % of Compound II.
62. The single tablet of any one of embodiments 38-60, wherein the second solid dispersion comprises 80 wt % of Compound II.
63. The single tablet of any one of embodiments 38-62, wherein the third solid dispersion comprises 75 wt % to 85 wt % of Compound III.
64. The single tablet of any one of embodiments 38-63, wherein the third solid dispersion comprises 80 wt % of Compound III.
65. The single tablet of any one of embodiments 38-64, comprising one or more excipients chosen from a filler, a disintegrant, a surfactant, and a lubricant.
66. The single tablet of embodiment 65, wherein the filler is chosen from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.
67. The single tablet of embodiment 65, wherein the disintegrant is chosen from croscarmellose sodium, sodium starch glycolate, crospovidone, corn or pre-gelatinized starch, sodium carboxymethyl cellulose, calcium carboxymethyl cellulose, and microcrystalline cellulose.
68. The single tablet of embodiment 65, wherein the lubricant is chosen from magnesium stearate, sodium stearyl fumarate, calcium stearate, sodium stearate, stearic acid, and talc, and wherein the surfactant is chosen from sodium lauryl sulfate, poloxamers, docusate sodium, PEGs and PEG derivatives.
69. The single tablet of any one of embodiments 38-68 wherein each of Compounds I, II and III is independently substantially amorphous.
70. The single tablet of embodiment 38 comprising an intra-granular part and extra-granular part, (a) wherein the intra-granular part comprises:
the first solid dispersion comprising said Compound I in 30 wt % to 40 wt % relative to the total weight of the tablet;
the second solid dispersion comprising said Compound II in 4 wt % to 8 wt % relative to the total weight of the tablet;
the third solid dispersion comprising said Compound III in 15 wt % to 20 wt % relative to the total weight of the tablet;
a disintegrant in 2 wt % to 6 wt % relative to the total weight of the tablet; and
(b) wherein the extra-granular part comprises:
a filler in 30 wt % to 40 wt % relative to the total weight of the tablet; and
a lubricant in 0.5 wt % to 1.5 wt % relative to the total weight of the tablet.
71. The single tablet of embodiment 38 comprising an intra-granular part and extra-granular part, (a) wherein the intra-granular part comprises:
the first solid dispersion comprising said Compound I in 36 wt % relative to the total weight of the tablet;
the second solid dispersion comprising said Compound II in 6 wt % relative to the total weight of the tablet;
the third solid dispersion comprising said Compound III in 18 wt % relative to the total weight of the tablet; and
a disintegrant in 4 wt % to 5 wt % relative to the total weight of the tablet; and
(b) wherein the extra-granular part comprises:
a filler in 34 wt % to 35 wt % relative to the total weight of the tablet; and
a lubricant in 1 wt % relative to the total weight of the tablet.
72. A single tablet comprising an intra-granular portion and an extra-granular portion, wherein either of the intra-granular portion or extra-granular position are comprised of a first solid dispersion comprising Compound I and a polymer, a second solid dispersion comprising Compound II and a polymer, and a third solid dispersion comprising Compound III and a polymer.
73. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 17 mg-20 mg croscarmellose sodium; and

(b) wherein the extra-granular part comprises:
(i) 286 mg-289 mg microcrystalline cellulose; and
(ii) 5 mg-7 mg magnesium stearate.
   74. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, relative to the total weight of the third solid dispersion, wherein the polymer is HPMCAS, and 0.5 wt % of sodium lauryl sulfate; and
(iv) 18 mg-21 mg croscarmellose sodium; and

(b) wherein the extra-granular part comprises:
(i) 108 mg-111 mg microcrystalline cellulose; and
(ii) 4 mg-6 mg magnesium stearate.
   75. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 36 mg-39 mg croscarmellose sodium; and

(b) wherein the extra-granular part comprises:
(i) 267 mg-270 mg microcrystalline cellulose; and
(ii) 5-8 mg magnesium stearate.
   76. The single tablet of embodiment 72,

(a) wherein the intra-granular part comprises:
(i) 186 mg-189 mg of a first solid dispersion of 80 wt % Compound I and 20 wt % of a polymer, wherein the polymer is HPMCAS, relative to the total weight of the first solid dispersion;
(ii) 30 mg-33 mg of a second solid dispersion of 80 wt % Compound II and 20 wt % of a polymer, wherein the polymer is HPMC, relative to the total weight of the second solid dispersion;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 27 mg-30 mg croscarmellose sodium; and

(b) wherein the extra-granular part comprises:
(i) 277 mg-280 mg microcrystalline cellulose; and
(ii) 5 mg-8 mg magnesium stearate.
   77. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 22 mg-25 mg croscarmellose sodium; and

(b) wherein the extra-granular part comprises:
(i) 273 mg-276 mg microcrystalline cellulose; and
(ii) 5 mg-8 mg magnesium stearate.
   78. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 22 mg-25 mg croscarmellose sodium; and

(b) wherein the extra-granular part comprises:
(i) 178 mg-181 mg microcrystalline cellulose; and
(ii) 4 mg-7 mg magnesium stearate.
   79. The single tablet of embodiment 72,

(a) wherein the intra-granular part comprises:
(i) 123 mg-127 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 17 mg-20 mg croscarmellose sodium; and

(b) wherein the extra-granular part comprises:
(i) 142 mg-145 mg microcrystalline cellulose; and
(ii) 3 mg-6 mg magnesium stearate.
   80. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion;
(iv) 152 mg-155 mg microcrystalline cellulose; and
(v) 27 mg-30 mg croscarmellose sodium; and

(b) wherein the extra-granular part comprises:
(i) 123 mg-127 mg microcrystalline cellulose; and
(ii) 5 mg-8 mg magnesium stearate.
   81. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 29 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 152 mg-155 mg microcrystalline cellulose; and

(b) wherein the extra-granular part comprises:
(i) 123 mg-127 mg microcrystalline cellulose;
(ii) 5 mg-8 mg magnesium stearate; and
(iii) 27 mg-30 mg croscarmellose sodium.
   82. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 29 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion;
(iv) 36 mg-39 mg croscarmellose sodium; and
(v) 5 mg-8 mg sodium lauryl sulfate; and

(b) wherein the extra-granular part comprises:
(i) 261 mg-264 mg microcrystalline cellulose; and
(ii) 5 mg-8 mg magnesium stearate.
   83. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 136 mg-139 mg microcrystalline cellulose; and
(iv) 27 mg-30 mg croscarmellose sodium; and

(b) wherein the extra-granular part comprises:
(i) 93 mg-96 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(ii) 375 mg-378 mg microcrystalline cellulose.
   84. The single tablet of embodiment 72,

(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 136 mg-139 mg microcrystalline cellulose; and
(iv) 27 mg-30 mg croscarmellose sodium; and

(b) wherein the extra-granular part comprises:
(i) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(ii) 280 mg-283 mg microcrystalline cellulose.
   85. The single tablet of embodiment 72,

(a) wherein the intra-granular part comprises:
(i) 30 mg-33 mg of a second solid dispersion of 80 wt % Compound II and 20 wt % of a polymer, relative to the total weight of the second solid dispersion, wherein the polymer is HPMC;
(ii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion;
(iii) 36 mg-39 mg croscarmellose sodium; and
(iv) 142 mg-145 mg microcrystalline cellulose; and

(b) wherein the extra-granular part comprises:
(i) 186 mg-189 mg of a first solid dispersion of 80 wt % Compound I and 20 wt % of a polymer, wherein the polymer is HPMCAS, relative to the total weight of the first solid dispersion;
(ii) 123 mg-127 mg microcrystalline cellulose; and
(iii) 5 mg-8 mg magnesium stearate.
   86. The single tablet of embodiment 72,

(a) wherein the intra-granular part comprises:
(i) 30 mg-33 mg of a second solid dispersion of 80 wt % Compound II and 20 wt % of a polymer, relative to the total weight of the second solid dispersion, wherein the polymer is HPMC;
(ii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iii) 36 mg-39 mg croscarmellose sodium; and

(b) wherein the extra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 267 mg-270 mg microcrystalline cellulose; and
(iii) 5 mg-8 mg magnesium stearate.
   87. A method of treating cystic fibrosis in a patient comprising orally administering to the patient the single tablet of any one of embodiments 1-86.
   88. The method of embodiment 87, wherein the single tablet is administered once daily.
   89. The method of embodiment 87, wherein the single tablet is administered twice daily.
   90. The method of embodiment 87, wherein two tablets are administered once daily.
   91. The method of embodiment 87, wherein two tablets are administered two times daily.
   92. The method according to any one of embodiments 87-91, wherein said patient has cystic fibrosis is chosen from patients with F508del/minimal function genotypes, patients with F508del/F508del genotypes, patients with F508del/gating genotypes, and patients with F508del/residual function genotypes.
   93. The method of embodiment 92, wherein the patient with a F508del/minimal function genotype has a minimal function mutation chosen from:

Mutation

| S4X | C276X | | G542X | R792X | E1104X |
|---|---|---|---|---|---|
| G27X | Q290X | | G550X | E822X | R1158X |
| Q39X | G330X | | Q552X | W846X | R1162X |
| W57X | W401X | | R553X | Y849X | S1196X |
| E60X | Q414X | | E585X | R851X | W1204X |
| R75X | S434X | | G673X | Q890X | L1254X |
| E92X | S466X | | Q685X | S912X | S1255X |
| Q98X | S489X | | R790X | Y913X | W1282X |
| Y122X | Q493X | | K710X | W1089X | Q1313X |
| E193X | W496X | | L732X | Y1092X | E1371X |
| L218X | C524X | | R764X | W1098X | Q1382X |
| Q220X | Q525X | | R785X | R1102X | Q1411X |
| 185+1G→T | 711+5G→A | | 1717−8G→A | 2622+1G→A | 3121−1G→A |
| 296+1G→A | 712−1G→T | | 1717−1G→A | 2790−1G→C | 3500−2A→G |
| 405+1G→A | 1248+1G→A | | 1811+1G→C | 3040G→C | 3600+2insT |
| 405+3A→C | 1249−1G→A | | 1811+1.6kbA→G | (G970R) | 3850−1G→A |
| 406−1G→A | 1341+1G→A | | 1812−1G→A | 3120G→A | 4005+1G→A |
| 621+1G→T | 1525−2A→G | | 1898+1G→A | 3120+1G→A | 4374+1G→T |
| 711+1G→T | 1525−1G→A | | 1898+1G→C | 3121−2A→G | |
| 182delT | 1119delA | | 1782delA | 2732insA | 3876delA |
| 306insA | 1138insG | | 1824delA | 2869insG | 3878delG |
| 365-366insT | 1154insTC | | 2043delG | 2896insAG | 3905insT |
| 394delTT | 1161delC | | 2143delT | 2942insT | 4016insT |
| 442delA | 1213delT | | 2183AA→G ^{a} | 2957delT | 4021dupT |
| 444delA | 1259insA | | 2184delA | 3007delG | 4040delA |
| 457TAT→G | 1288insTA | | 2184insA | 3028delA | 4279insA |
| 541delC | 1471delA | | 2307insA | 3171delC | 4326delTC |
| 574delA | 1497delGG | | 2347delG | 3659delC | |
| 663delT | 1548delG | | 2585delT | 3737delA | |
| 935delA | 1609del CA | | 2594delGT | 3791delC | |
| 1078delT | 1677delTA | | 2711delT | 3821delT | |
| CFTRdele2,3 | | 1461ins4 | | 2991del32 | |
| CFTRdele22,23 | | 1924del7 | | 3199del6^{c} | |
| 124del23bp | | 2055del9→A | | 3667ins4 | |
| 852del22 | | 2105-2117del13insAGAAA | | 4010del4 | |
| 991del5 | | 2721del11 | | 4209TGTT→AA | |
| A46D^{b} | V520F | | Y569D^{b} | N1303K | |
| G85E | A559T^{b} | | L1065P | | |
| R347P | R560T | | R1066C | | |
| L467P^{b} | R560S | | L1077P^{b} | | |
| I507del | A561E | | M1101K | | |

94. The method of embodiment 92, wherein the patient with a F508del/gating genotype has a gating mutation chosen from G178R, S549N, S549R, G551D, G551S, G1244E, S1251N, S1255P, and G1349D.
95. The method of embodiment 92, wherein the patient with a F508del/residual function genotype has a residual function mutation chosen from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.

Methods of Preparing Compounds and Tablets

General Experimental Procedures

Reagents and starting materials were obtained by commercial sources unless otherwise stated and were used without purification. Proton and carbon NMR spectra were acquired on either of a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

Final purity of compounds was determined by reversed phase UPLC using an Acquity UPLC BEH C18 column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+H]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30m×0.25 mm×0.25 um_df) column, with a 2.0 mL/min flow rate (H₂ carrier gas), at an injection temperature of 220° C. and an oven temperature of 120° C., 15 minutes.

Solid state ¹³C and ¹⁹F NMR data was obtained using Bruker-Biospin 400 MHz wide-bore spectrometer equipped with Bruker-Biospin 4 mm HFX probe was used. Samples were packed into 4 mm rotors and spun under Magic Angle Spinning (MAS) condition with typical spinning speed of 12.5 kHz. The proton relaxation time was estimated from ¹H MAS T₁ saturation recovery relaxation experiment and used to set up proper recycle delay of the ¹³C cross-polarization (CP) MAS experiment. The fluorine relaxation time was estimated from ¹⁹F MAS T₁ saturation recovery relaxation experiment and used to set up proper recycle delay of the ¹⁹F MAS experiment. The CP contact time of CPMAS experiments was set to 2 ms. A CP proton pulse with linear ramp (from 50% to 100%) was employed. All spectra were externally referenced by adjusting the magnetic field to set carbon resonance of adamantane to 29.5 ppm. TPPM15 proton decoupling sequence was used with the field strength of approximately 100 kHz for both ¹³C and ¹⁹F acquisitions.

Final purity of compounds was determined by reversed phase UPLC using an Acquity UPLC BEH C18 column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+H]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30m×0.25 mm×0.25 um_df) column, with a 2.0 mL/min flow rate (H₂ carrier gas), at an injection temperature of 220° C. and an oven temperature of 120° C., 15 minutes.

### Example 1. Synthesis of Compound I: N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide

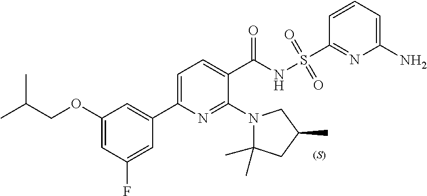

### Step 1: tert-butyl 2-chloro-6-(3-fluoro-5-isobutoxy-phenyl)pyridine-3-carboxylate

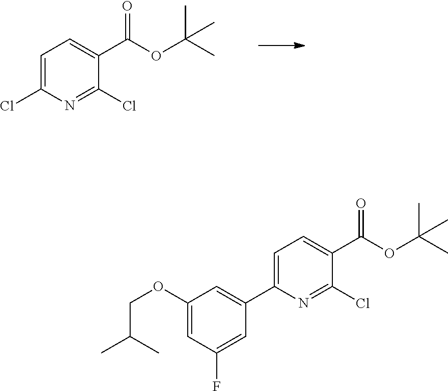 [SMILES: CC(C)(C)OC(=O)c1ccc(Cl)nc1Cl ; CC(C)COc1cc(F)cc(-c2ccc(C(=O)OC(C)(C)C)c(Cl)n2)c1]

tert-Butyl 2,6-dichloropyridine-3-carboxylate (15.0 g, 60.5 mmol) and (3-fluoro-5-isobutoxy-phenyl)boronic acid (13.46 g, 63.48 mmol) were combined and fully dissolved in ethanol (150 mL) and toluene (150 mL). A suspension of sodium carbonate (19.23 g, 181.4 mmol) in water (30 mL) was added. Tetrakis(triphenylphosphine)palladium (0) (2.096 g, 1.814 mmol) was added under nitrogen. The reaction mixture was allowed to stir at 60° C. for 16 hours. Volatiles were removed under reduced pressure. The remaining solids were partitioned between water (100 mL) and ethyl acetate (100 mL). The organic layer was washed with brine (1×100 mL), dried over sodium sulfate, filtered, and concentrated under reduced pressure. The material was subjected silica gel column chromatography on a 330 gram silica gel column, 0 to 20% ethyl acetate in hexanes gradient. The material was repurified on a 220 gram silica gel column, isocratic 100% hexane for 10 minutes, then a 0 to 5% ethyl acetate in hexanes gradient to yield tert-butyl 2-chloro-6-(3-fluoro-5-isobutoxy-phenyl)pyridine-3-carboxylate (18.87 g, 49.68 mmol, 82.2%) as a colorless oil. ¹H NMR (400 MHz, DMSO-d₆) δ 8.24 (d, J=8.0 Hz, 1H), 8.16 (d, J=8.1 Hz, 1H), 7.48 (dd, J=9.4, 2.0 Hz, 2H), 6.99 (dt, J=10.8, 2.2 Hz, 1H), 3.86 (d, J=6.5 Hz, 2H), 2.05 (dt, J=13.3, 6.6 Hz, 1H), 1.57 (d, J=9.3 Hz, 9H), 1.00 (t, J=5.5 Hz, 6H). ESI-MS m/z calc. 379.13504, found 380.2 (M+1)⁺; Retention time: 2.57 minutes.

### Step 2: 2-chloro-6-(3-fluoro-5-isobutoxy-phenyl)pyridine-3-carboxylic acid

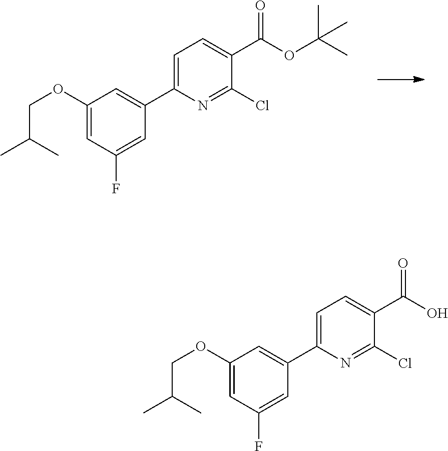

tert-Butyl 2-chloro-6-(3-fluoro-5-isobutoxy-phenyl)pyridine-3-carboxylate (18.57 g, 48.89 mmol) was dissolved in dichloromethane (200 mL). Trifluoroacetic acid (60 mL, 780 mmol) was added and the reaction mixture was allowed to stir at room temperature for 1 hour. The reaction mixture was stirred at 40° C. for 2 hours. The reaction mixture was concentrated under reduced pressure and taken up in ethyl acetate (100 mL). It was washed with a saturated aqueous sodium bicarbonate solution (1×100 mL) and brine (1×100 mL), dried over sodium sulfate, filtered, and concentrated under reduced pressure. The crude product was suspended in ethyl acetate (75 mL) and washed with aqueous HCl (1 N, 1×75 mL). The organic layer was dried over sodium sulfate, filtered and concentrated under reduced pressure. The remaining solid (17.7 g) was stirred as a slurry in dichloromethane (35 mL) at 40° C. for 30 minutes. After cooling to room temperature, the remaining slurry was filtered, and then rinsed with cold dichloromethane to give 2-chloro-6-(3-fluoro-5-isobutoxy-phenyl)pyridine-3-carboxylic acid (11.35 g, 35.06 mmol, 72%) as a white solid. ¹H NMR (400 MHz, DMSO-d₆) δ 13.76 (s, 1H), 8.31 (d, J=8.0 Hz, 1H), 8.17 (d, J=8.1 Hz, 1H), 7.54-7.47 (m, 2H), 7.00 (dt, J=10.8, 2.3 Hz, 1H), 3.87 (d, J=6.5 Hz, 2H), 2.05 (dt, J=13.3, 6.6 Hz, 1H), 1.01 (d, J=6.7 Hz, 6H). ESI-MS m/z calc. 323.1, found 324.1 (M+1)⁺; Retention time: 1.96 minutes.

### Step 3: N-[(6-amino-2-pyridyl)sulfonyl]-2-chloro-6-(3-fluoro-5-isobutoxy-phenyl)pyridine-3-carboxamide

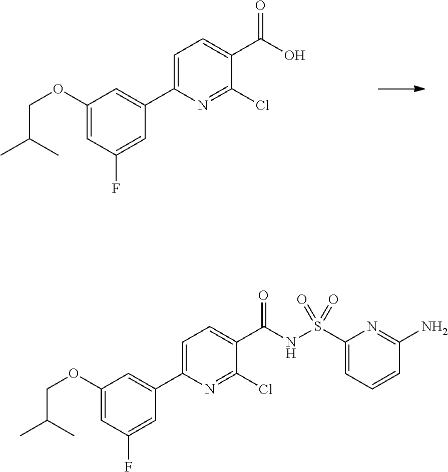 [SMILES: CC(C)COc1cc(F)cc(-c2ccc(C(=O)O)c(Cl)n2)c1 ; CC(C)COc1cc(F)cc(-c2ccc(C(=O)NS(=O)(=O)c3cccc(N)n3)c(Cl)n2)c1]

2-Chloro-6-(3-fluoro-5-isobutoxy-phenyl)pyridine-3-carboxylic acid (3.00 g, 9.27 mmol) was dissolved in N,N-dimethylformamide (30.00 mL), and 1,1′-carbonyldiimidazole (2.254 g, 13.90 mmol) was added to the solution. The solution was allowed to stir at 65° C. for 1 hour. In a separate flask, sodium hydride (444.8 mg, 11.12 mmol) was added to a solution of 6-aminopyridine-2-sulfonamide (1.926 g, 11.12 mmol) in N,N-dimethylformamide (15.00 mL). This mixture was stirred for one hour before being added to the prior reaction mixture. The final reaction mixture was stirred at 65° C. for 15 minutes. Volatiles were removed under reduced pressure. The remaining oil was taken up in ethyl acetate and washed with aqueous HCl (1 N, 1×75 mL) and brine (3×75 mL). The organic layer was dried over sodium sulfate, filtered, and concentrated under reduced pressure. The remaining white solid (4.7 g) was fully dissolved in isopropanol (120 mL) in an 85° C. water bath. The colorless solution was allowed to slowly cool to room temperature with slow stirring over 16 hours. The crystalline solids that had formed were collected by vacuum filtration, and then rinsed with cold isopropanol (50 mL). Upon drying, N-[(6-amino-2-pyridyl)sulfonyl]-2-chloro-6-(3-fluoro-5-isobutoxy-phenyl)pyridine-3-carboxamide (3.24 g, 6.765 mmol, 73%) was obtained as a white solid. ¹H NMR (400 MHz, DMSO-d₆) δ 12.78 (s, 1H), 8.15 (d, J=8.0 Hz, 1H), 8.09 (d, J=7.9 Hz, 1H), 7.73-7.63 (m, 1H), 7.49 (dd, J=8.6, 1.9 Hz, 2H), 7.21 (d, J=7.3 Hz, 1H), 6.99 (dt, J=10.7, 2.2 Hz, 1H), 6.74 (d, J=8.4 Hz, 1H), 6.64 (s, 2H), 3.86 (d, J=6.5 Hz, 2H), 2.05 (dp, J=13.3, 6.5 Hz, 1H), 1.02 (dd, J=12.7, 6.4 Hz, 6H).

### Step 4: N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) and N-[(6-amino-2-pyridyl)sulfonyl]-6-(3-fluoro-5-isobutoxy-phenyl)-2-[(4R)-2,2,4-trimethylpyrrolidin-1-yl]pyridine-3-carboxamide

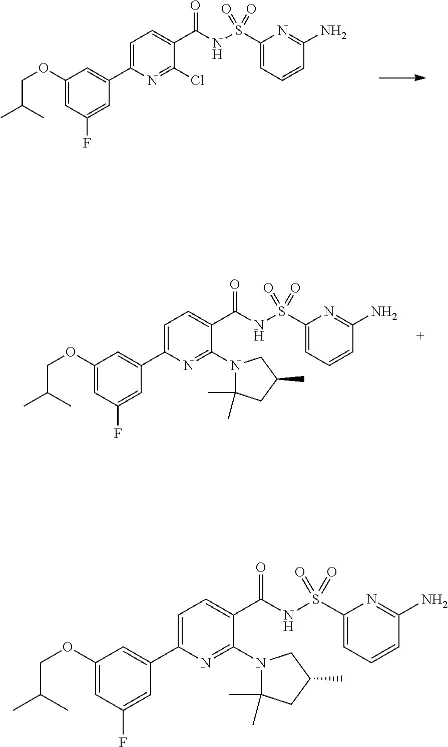 [SMILES: CC(C)COc1cc(F)cc(-c2ccc(C(=O)NS(=O)(=O)c3cccc(N)n3)c(Cl)n2)c1 ; CC(C)COc1cc(F)cc(-c2ccc(C(=O)NS(=O)(=O)c3cccc(N)n3)c(N3C[C@@H](C)CC3(C)C)n2)c1 ; CC(C)COc1cc(F)cc(-c2ccc(C(=O)NS(=O)(=O)c3cccc(N)n3)c(N3C[C@H](C)CC3(C)C)n2)c1]

N-[(6-Amino-2-pyridyl)sulfonyl]-2-chloro-6-(3-fluoro-5-isobutoxy-phenyl)pyridine-3-carboxamide (309 mg, 0.645 mmol) was dissolved in dimethylsulfoxide (3.708 mL) and potassium carbonate (445.9 mg, 3.226 mmol) was slowly added, followed by 2,2,4-trimethylpyrrolidine (146.0 mg, 1.290 mmol). The reaction mixture was sealed and heated at 150° C. for 72 hours. The reaction was cooled down, diluted with water (50 mL), extracted 3 times with 50 mL portions of ethyl acetate, washed with brine, dried over sodium sulfate, filtered and evaporated to dryness. The crude material was dissolved in 2 mL of dichloromethane and purified by on silica gel using a gradient of 0 to 80% ethyl acetate in hexanes. The stereoisomers were separated using supercritical fluid chromatography on a ChiralPak AD-H (250×4.6 mm), 5 m column using 25% isopropanol with 1.0% diehtylamine in CO₂ at a flow rate of 3.0 mL/min. The separated enantiomers were separately concentrated, diluted with ethyl acetate (3 mL) and washed with 1N aqueous hydrochloric acid. The organic layers were dried over sodium sulfate, filtered, and evaporated to dryness to give the pure compounds as pale yellow solids.

The first compound to elute from the SFC conditions given above gave N-[(6-amino-2-pyridyl)sulfonyl]-6-(3-fluoro-5-isobutoxy-phenyl)-2-[(4R)-2,2,4-trimethylpyrrolidin-1-yl]pyridine-3-carboxamide (Hydrochloric Acid)¹H NMR (400 MHz, DMSO-d6) δ 12.47 (s, 1H), 7.78 (d, J=8.0 Hz, 1H), 7.69-7.57 (m, 1H), 7.56-7.46 (m, 1H), 7.41 (dt, J=10.1, 1.8 Hz, 1H), 7.26 (d, J=8.0 Hz, 1H), 7.21 (d, J=7.2 Hz, 1H), 6.89 (dt, J=10.7, 2.3 Hz, 1H), 6.69 (d, J=8.3 Hz, 1H), 3.83 (d, J=6.7 Hz, 2H), 2.61 (dq, J=9.7, 4.9 Hz, 2H), 2.24 (d, J=15.8 Hz, 1H), 2.06 (dq, J=13.3, 6.7 Hz, 1H), 1.93-1.82 (m, 1H), 1.61 (s, 3H), 1.59 (s, 3H), 1.48-1.33 (m, 1H), 1.32-1.20 (m, 2H), 0.99 (d, J=6.6 Hz, 6H), 0.88 (d, J=6.2 Hz, 3H). ESI-MS m/z calc. 555.2, found 556.4 (M+1)⁺; Retention time: 2.76 minutes.

The second compound to elute from the SFC conditions described above gave N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) (Hydrochloric Acid (1)) ¹H NMR (400 MHz, Chloroform-d) δ 15.49 (s, 1H), 8.49 (d, J=8.2 Hz, 1H), 7.75-7.56 (m, 3H), 7.34 (t, J=1.8 Hz, 1H), 7.30 (dt, J=9.4, 1.9 Hz, 1H), 6.75-6.66 (m, 2H), 3.95 (s, 1H), 3.78 (d, J=6.5 Hz, 2H), 3.42 (s, 1H), 2.88-2.74 (m, 1H), 2.23 (dd, J=12.5, 8.0 Hz, 1H), 2.17-2.08 (m, 1H), 1.98-1.87 (m, 1H), 1.55 (s, 3H), 1.39 (s, 3H), 1.31 (d, J=6.7 Hz, 3H), 1.05 (d, J=6.7 Hz, 6H). ESI-MS m/z calc. 555.2, found 556.4 (M+1)⁺; Retention time: 2.77 minutes. Absolute stereochemistry was confirmed by X-ray crystallography.

### Example 2: Synthesis of Compound: (R)-1-(2,2-Difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

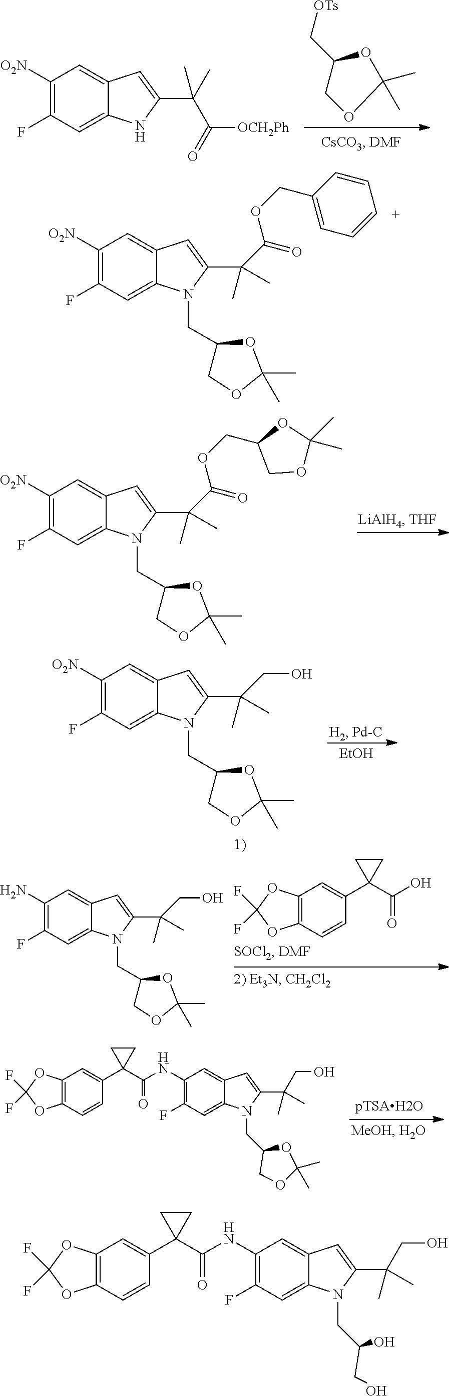

### Step 1: (R)-Benzyl 2-(1-((2,2-dimethyl-1,3-dioxolan-4-yl)methyl)-6-fluoro-5-nitro-1H-indol-2-yl)-2-methylpropanoate and ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate

Cesium carbonate (8.23 g, 25.3 mmol) was added to a mixture of benzyl 2-(6-fluoro-5-nitro-1H-indol-2-yl)-2-methylpropanoate (3.0 g, 8.4 mmol) and (S)-(2,2-dimethyl-1,3-dioxolan-4-yl)methyl 4-methylbenzenesulfonate (7.23 g, 25.3 mmol) in DMF (N,N-dimethylformamide) (17 mL). The reaction was stirred at 80° C. for 46 hours under a nitrogen atmosphere. The mixture was then partitioned between ethyl acetate and water. The aqueous layer was extracted with ethyl acetate. The combined ethyl acetate layers were washed with brine, dried over MgSO₄, filtered and concentrated. The crude product, a viscous brown oil which contains both of the products shown above, was taken directly to the next step without further purification. (R)-Benzyl 2-(1-((2,2-dimethyl-1,3-dioxolan-4-yl)methyl)-6-fluoro-5-nitro-1H-indol-2-yl)-2-methylpropanoate, ESI-MS m/z calc. 470.2, found 471.5 (M+1)⁺. Retention time 2.20 minutes. ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate, ESI-MS m/z calc. 494.5, found 495.7 (M+1)⁺. Retention time 2.01 minutes.

### Step 2: (R)-2-(1-((2,2-dimethyl-1,3-dioxolan-4-yl)methyl)-6-fluoro-5-nitro-1H-indol-2-yl)-2-methylpropan-1-ol

The crude reaction mixture obtained in step (A) was dissolved in THF (tetrahydrofuran) (42 mL) and cooled in an ice-water bath. LiAlH₄ (16.8 mL of 1 M solution, 16.8 mmol) was added drop-wise. After the addition was complete, the mixture was stirred for an additional 5 minutes. The reaction was quenched by adding water (1 mL), 15% NaOH solution (1 mL) and then water (3 mL). The mixture was filtered over Celite, and the solids were washed with THF and ethyl acetate. The filtrate was concentrated and purified by column chromatography (30-60% ethyl acetate-hexanes) to obtain (R)-2-(1-((2,2-dimethyl-1,3-dioxolan-4-yl)methyl)-6-fluoro-5-nitro-1H-indol-2-yl)-2-methylpropan-1-ol as a brown oil (2.68 g, 87% over 2 steps). ESI-MS m/z calc. 366.4, found 367.3 (M+1)⁺. Retention time 1.68 minutes. ¹H NMR (400 MHz, DMSO-d6) δ 8.34 (d, J=7.6 Hz, 1H), 7.65 (d, J=13.4 Hz, 1H), 6.57 (s, 1H), 4.94 (t, J=5.4 Hz, 1H), 4.64-4.60 (m, 1H), 4.52-4.42 (m, 2H), 4.16-4.14 (m, 1H), 3.76-3.74 (m, 1H), 3.63-3.53 (m, 2H), 1.42 (s, 3H), 1.38-1.36 (m, 6H) and 1.19 (s, 3H) ppm. (DMSO is dimethylsulfoxide).

### Step 3: (R)-2-(5-amino-1-((2,2-dimethyl-1,3-dioxolan-4-yl)methyl)-6-fluoro-1H-indol-2-yl)-2-methylpropan-1-ol

(R)-2-(1-((2,2-dimethyl-1,3-dioxolan-4-yl)methyl)-6-fluoro-5-nitro-1H-indol-2-yl)-2-methylpropan-1-ol (2.5 g, 6.82 mmol) was dissolved in ethanol (70 mL) and the reaction was flushed with N₂. Then Pd—C (250 mg, 5% wt) was added. The reaction was flushed with nitrogen again and then stirred under H₂ (atm). After 2.5 hours only partial conversion to the product was observed by LCMS. The reaction was filtered through Celite and concentrated. The residue was re-subjected to the conditions above. After 2 hours LCMS indicated complete conversion to product. The reaction mixture was filtered through Celite. The filtrate was concentrated to yield the product (1.82 g, 79%). ESI-MS m/z calc. 336.2, found 337.5 (M+1)⁺. Retention time 0.86 minutes. ¹H NMR (400 MHz, DMSO-d6) δ 7.17 (d, J=12.6 Hz, 1H), 6.76 (d, J=9.0 Hz, 1H), 6.03 (s, 1H), 4.79-4.76 (m, 1H), 4.46 (s, 2H), 4.37-4.31 (m, 3H), 4.06 (dd, J=6.1, 8.3 Hz, 1H), 3.70-3.67 (m, 1H), 3.55-3.52 (m, 2H), 1.41 (s, 3H), 1.32 (s, 6H) and 1.21 (s, 3H) ppm.

### Step 4: (R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide

DMF (3 drops) was added to a stirring mixture of 1-(2,2-difluorobenzo[d][1,3]dioxol-5-yl)cyclopropanecarboxylic acid (1.87 g, 7.7 mmol) and thionyl chloride (1.30 mL, 17.9 mmol). After 1 hour a clear solution had formed. The solution was concentrated under vacuum and then toluene (3 mL) was added and the mixture was concentrated again. The toluene step was repeated once more and the residue was placed on high vacuum for 10 minutes. The acid chloride was then dissolved in dichloromethane (10 mL) and added to a mixture of (R)-2-(5-amino-1-((2,2-dimethyl-1,3-dioxolan-4-yl)methyl)-6-fluoro-1H-indol-2-yl)-2-methylpropan-1-ol (1.8 g, 5.4 mmol) and triethylamine (2.24 mL, 16.1 mmol) in dichloromethane (45 mL). The reaction was stirred at room temperature for 1 hour. The reaction was washed with 1N HCl solution, saturated NaHCO₃solution and brine, dried over MgSO₄ and concentrated to yield the product (3 g, 100%). ESI-MS m/z calc. 560.6, found 561.7 (M+1)⁺. Retention time 2.05 minutes. ¹H NMR (400 MHz, DMSO-d6) δ 8.31 (s, 1H), 7.53 (s, 1H), 7.42-7.40 (m, 2H), 7.34-7.30 (m, 3H), 6.24 (s, 1H), 4.51-4.48 (m, 1H), 4.39-4.34 (m, 2H), 4.08 (dd, J=6.0, 8.3 Hz, 1H), 3.69 (t, J=7.6 Hz, 1H), 3.58-3.51 (m, 2H), 1.48-1.45 (m, 2H), 1.39 (s, 3H), 1.34-1.33 (m, 6H), 1.18 (s, 3H) and 1.14-1.12 (m, 2H) ppm

### Step 5: (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

(R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide (3.0 g, 5.4 mmol) was dissolved in methanol (52 mL). Water (5.2 mL) was added followed by p-TsOH.H₂O (p-toluenesulfonic acid hydrate) (204 mg, 1.1 mmol). The reaction was heated at 80° C. for 45 minutes. The solution was concentrated and then partitioned between ethyl acetate and saturated NaHCO₃solution. The ethyl acetate layer was dried over MgSO₄ and concentrated. The residue was purified by column chromatography (50-100% ethyl acetate-hexanes) to yield the product. (1.3 g, 47%, ee>98% by SFC). ESI-MS m/z calc. 520.5, found 521.7 (M+1)⁺. Retention time 1.69 minutes. ¹H NMR (400 MHz, DMSO-d6) δ 8.31 (s, 1H), 7.53 (s, 1H), 7.42-7.38 (m, 2H), 7.33-7.30 (m, 2H), 6.22 (s, 1H), 5.01 (d, J=5.2 Hz, 1H), 4.90 (t, J=5.5 Hz, 1H), 4.75 (t, J=5.8 Hz, 1H), 4.40 (dd, J=2.6, 15.1 Hz, 1H), 4.10 (dd, J=8.7, 15.1 Hz, 1H), 3.90 (s, 1H), 3.65-3.54 (m, 2H), 3.48-3.33 (m, 2H), 1.48-1.45 (m, 2H), 1.35 (s, 3H), 1.32 (s, 3H) and 1.14-1.11 (m, 2H) ppm.

### Example 3: Synthesis of Compound III: N-(2,4-di-tert-butyl-5-hydroxyphenyl)-4-oxo-1,4-dihydroquinoline-3-carboxamide

### Part A: Synthesis of 4-oxo-1,4-dihydroquinoline-3-carboxylic acid

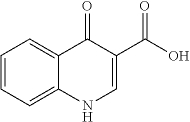 [SMILES: O=C(O)c1c[nH]c2ccccc2c1=O]

### Step 1: 2-Phenylaminomethylene-malonic acid diethyl ester

A mixture of aniline (25.6 g, 0.275 mol) and diethyl 2-(ethoxymethylene)malonate (62.4 g, 0.288 mol) was heated at 140-150° C. for 2 h. The mixture was cooled to room temperature and dried under reduced pressure to afford 2-phenylaminomethylene-malonic acid diethyl ester as a solid, which was used in the next step without further purification. ¹H NMR (DMSO-d₆) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H).

### Step 2: 4-Hydroxyquinoline-3-carboxylic acid ethyl ester

A 1 L three-necked flask fitted with a mechanical stirrer was charged with 2-phenylaminomethylene-malonic acid diethyl ester (26.3 g, 0.100 mol), polyphosphoric acid (270 g) and phosphoryl chloride (750 g). The mixture was heated to 70° C. and stirred for 4 h. The mixture was cooled to room temperature and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in next step without further purification.

### Step 3: 4-Oxo-1,4-dihydroquinoline-3-carboxylic acid

4-Hydroxyquinoline-3-carboxylic acid ethyl ester (15 g, 69 mmol) was suspended in sodium hydroxide solution (2N, 150 mL) and stirred for 2 h at reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid as a pale white solid (10.5 g, 92%). ¹H NMR (DMSO-d₆) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H).

### Part B: Synthesis of N-(2,4-di-tert-butyl-5-hydroxyphenyl)-4-oxo-1,4-dihydroquinoline-3-carboxamide

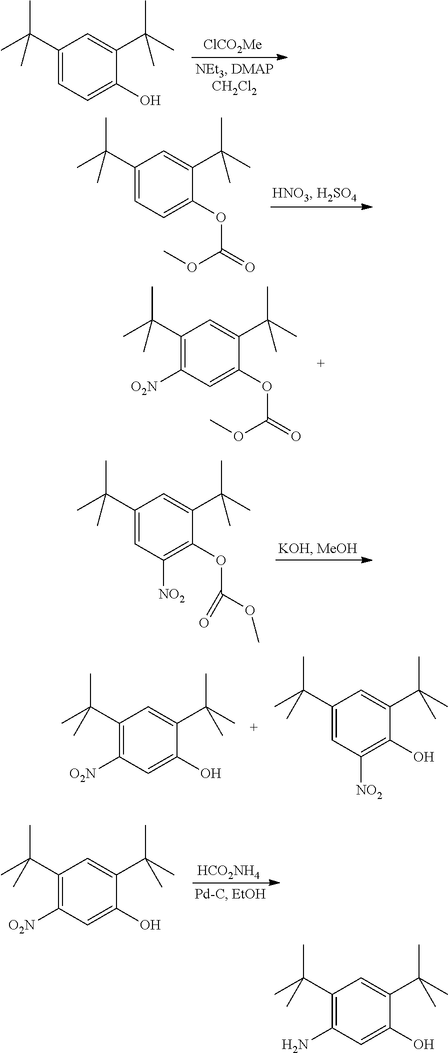

### Step 1: Carbonic acid 2,4-di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1 L) using 10% ethyl acetate-hexanes (4 L) as the eluent. The combined filtrates were concentrated to yield carbonic acid 2,4-di-tert-butyl-phenyl ester methyl ester as a yellow oil (132 g, quant.). ¹H NMR (400 MHz, DMSO-d₆) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H).

### Step 2: Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 180 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate-hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### Step 3: 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 14.0 mmol) was dissolved in MeOH (65 mL) before KOH (2.0 g, 36 mmol) was added. The mixture was stirred at room temperature for 2 h. The reaction mixture was then made acidic (pH 2-3) by adding conc. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d₆) δ 10.14 (s, 1H, OH), 7.34 (s, 1H), 6.83 (s, 1H), 1.36 (s, 9H), 1.30 (s, 9H). 2,4-Di-tert-butyl-6-nitro-phenol: ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H).

### Step 4: 5-Amino-2,4-di-tert-butyl-phenol

To a refluxing solution of 2,4-di-tert-butyl-5-nitro-phenol (1.86 g, 7.40 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d₆) δ 8.64 (s, 1H, OH), 6.84 (s, 1H), 6.08 (s, 1H), 4.39 (s, 2H, NH₂), 1.27 (m, 18H); HPLC ret. time 2.72 min, 10-99% CH₃CN, 5 min run; ESI-MS 222.4 m/z [M+H]⁺.

### Step 5: N-(5-hydroxy-2,4-di-tert-butyl-phenyl)-4-oxo-1H-quinoline-3-carboxamide

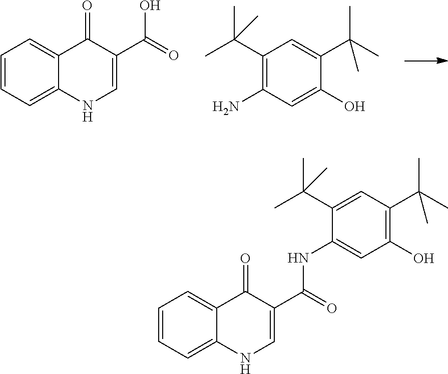

To a suspension of 4-oxo-1,4-dihydroquinolin-3-carboxylic acid (35.5 g, 188 mmol) and HBTU (85.7 g, 226 mmol) in DMF (280 mL) was added Et₃N (63.0 mL, 451 mmol) at ambient temperature. The mixture became homogeneous and was allowed to stir for 10 min before 5-amino-2,4-di-tert-butyl-phenol (50.0 g, 226 mmol) was added in small portions. The mixture was allowed to stir overnight at ambient temperature. The mixture became heterogeneous over the course of the reaction. After all of the acid was consumed (LC-MS analysis, MH+ 190, 1.71 min), the solvent was removed in vacuo. EtOH (ethyl alcohol) was added to the orange solid material to produce a slurry. The mixture was stirred on a rotovap (bath temperature 65° C.) for 15 min without placing the system under vacuum. The mixture was filtered and the captured solid was washed with hexanes to provide a white solid that was the EtOH crystalate. Et₂O (diethyl ether) was added to the solid obtained above until a slurry was formed. The mixture was stirred on a rotovapor (bath temperature 25° C.) for 15 min without placing the system under vacuum. The mixture was filtered and the solid captured. This procedure was performed a total of five times. The solid obtained after the fifth precipitation was placed under vacuum overnight to provide N-(5-hydroxy-2,4-di-tert-butyl-phenyl)-4-oxo-1H-quinoline-3-carboxamide (38 g, 52%). HPLC ret. time 3.45 min, 10-99% CH₃CN, 5 min run; ¹H NMR (400 MHz, DMSO-d₆) δ 12.88 (s, 1H), 11.83 (s, 1H), 9.20 (s, 1H), 8.87 (s, 1H), 8.33 (dd, J=8.2, 1.0 Hz, 1H), 7.83-7.79 (m, 1H), 7.76 (d, J=7.7 Hz, 1H), 7.54-7.50 (m, 1H), 7.17 (s, 1H), 7.10 (s, 1H), 1.38 (s, 9H), 1.37 (s, 9H); ESI-MS m/z calc'd 392.21; found 393.3 [M+H]⁺.

### Example 4: Preparation of a Solid Dispersion Comprising Substantially Amorphous Compound I and HPMCAS-H Polymer

A solvent system of dichloromethane (DCM) and methanol (MeOH), was formulated according to the ratio 80.0 wt % DCM/20.0 wt % MeOH, in an appropriately sized container, and mixed using overhead mixer and propeller-type blade. Into this solvent system, hypromellose acetate succinate polymer (HPMCAS, H grade) and Compound I were added according to the ratio 20 wt % hypromellose acetate succinate/80 wt % Compound I. The resulting mixture contained 15.0 wt % solids. The actual amounts of ingredients and solvents used to generate this mixture are recited in Table 7, below:

TABLE 7
Solid spray dispersion ingredients for amorphous Compound I.

| | Units | Batch |
|---|---|---|
| Compound I | g | 1000.0 |
| HPMCAS | g | 250.0 |
| Total Solids | g | 1250.0 |
| DCM | g | 5666.7 |
| MeOH | g | 1416.7 |
| Total Solvents | g | 7083.4 |
| Total Spray Solution Weight | g | 8333.4 |

The mixture was mixed until it was substantially homogenous and all components were substantially dissolved.

A spray drier, Anhydro MS-35 Spray Drier, fitted with two fluid 0.8 mm nozzle (Schlick series 970/0 S4), was used under normal spray drying mode, following the dry spray process parameters recited in Table 8.

TABLE 8
Spray drying dispersion processing parameters to generate
solid spray dispersion of amorphous Compound I.

| Parameter: | Value: | |
|---|---|---|
| Process Gas Flow Rate | 35 | Kg/hr |
| Nozzle Gas Flow Rate | 4.3 | Kg/hr |
| Feed Flow Rate | 2 | Kg/hr |
| Inlet Temperature | 70-75° | C. |
| Outlet Temperature | 36-46° | C. |
| Vacuum Dryer Temperature | 40° | C. |

| | |
|---|---|
| Vacuum Drying Time | No less than 24 hours |

A high efficiency cyclone separated the wet product from the spray gas and solvent vapors. The wet product was transferred into trays and placed in vacuum dryer for drying to reduce residual solvents to a level of less than 3000 ppm for MeOH and less than 600 ppm of DCM and to generate dry spray dry dispersion of amorphous Compound I, containing <0.01% MeOH and <0.01% DCM.

### Example 5: Preparation of Tablet Formulation A

Screening/Weighing:

The solid dispersion comprising 80 wt % substantially amorphous Compound I and 20 wt % HPMCAS as shown in Example 4, the solid dispersion comprising 80 wt % substantially amorphous Compound II and 20 wt % HPMC (see PCT Publication No. WO 2015/160787, the entire contents are incorporated herein by reference), the solid dispersion comprising 80 wt % substantially amorphous Compound III, 19.5 wt % HPMCAS and 0.5 wt % sodium lauryl sulfate (see WO 2015/160787), and excipients (see Table 9) were screened prior to or after weigh-out. Screen sizes used were mesh #20 for all components except magnesium stearate which used mesh #60.

Blending:

The solid dispersion comprising substantially amorphous Compound I, the solid dispersion comprising substantially amorphous Compound II, and solid dispersion comprising substantially amorphous Compound III, and croscarmellose sodium were blended. The blending was performed using a bin blender. The components were blended for 5 minutes.

Dry Granulation:

The blend was granulated using a Gerteis roller compactor using combined smooth/knurled rolls and an integrated 1.0 mm mesh milling screen with pocketed rotor and paddle agitator. The roller compactor was operated with a roll gap of 2 mm, roll pressure of 4.5 kNcm, roll speed of 2 rpm, granulation speed of 80/80 (CW/CCW) rpm, and oscillation of 330/360 (CW/CCW) degrees.

Blending:

The roller compacted granules were blended with microcrystalline cellulose using a bin blender. The blending time was 4 minutes. Magnesium stearate was added to bin and further blended for an additional 2 minutes.

Compression:

The compression blend was compressed into tablets using a Riva Piccola rotary tablet press. The weight of the tablets for a dose of 150 mg of substantially amorphous Compound I, 25.0 mg of substantially amorphous Compound II, and 75 mg of substantially amorphous Compound III was 521 mg.

Coating:

The core tablets were film coated using an Ohara tablet film coater. The film coat suspension was prepared by adding the coating material to purified water. The required amount of film coating suspension (3% of the tablet weight) was sprayed onto the tablets to achieve the desired weight gain.

TABLE 9
Tablet “A” Comprising 150 mg Compound I,
25.0 mg Compound II and 75 mg Compound III.

| | | Amount per |
|---|---|---|
| | Ingredient | tablet (mg) |
| Intra-granular | Compound I SDD (80 wt % | 187.5 |
| | Compound I and 20 wt % | |
| | HPMCAS) | |
| | Compound II SDD (80 wt % | 31.2 |
| | Compound II and 20 wt % | |
| | HPMC) | |
| | Compound III SDD (80 wt % | 93.8 |
| | Compound III, 19.5 wt % | |
| | HPMCAS, and 0.5 wt % | |
| | sodium lauryl sulfate) | |
| | Croscarmellose Sodium | 23.5 |
| | Total | 335.9 |
| Extra-granular | Microcrystalline cellulose | 179.7 |
| | Magnesium Stearate | 5.2 |
| | Total | 184.9 |
| Total uncoated Tablet | | 520.8 |
| Film coat | Opadry | 15.6 |
| Total coated Tablet | | 536.4 |

### Example 6: Preparation of Tablet Formulation B

Screening/Weighing:

The solid dispersion comprising 80 wt % substantially amorphous Compound I and 20 wt % HPMCAS as shown in Example 4, the solid dispersion comprising 80 wt % substantially amorphous Compound II and 20 wt % HPMC (see PCT Publication No. WO 2015/160787, the entire contents are incorporated herein by reference), the solid dispersion comprising 80 wt % substantially amorphous Compound III, 19.5 wt % HPMCAS and 0.5 wt % sodium lauryl sulfate (see WO 2015/160787), and excipients (see Table 10) were screened prior to or after weigh-out. Screen sizes used were mesh #20 for all components except magnesium stearate which used mesh #60.

Blending:

The solid dispersion comprising substantially amorphous Compound I, the solid dispersion comprising substantially amorphous Compound II, and solid dispersion comprising substantially amorphous Compound III, and croscarmellose sodium were blended. The blending was performed using a bin blender. The components were blended for 5 minutes.

Dry Granulation:

The blend was granulated using a Gerteis roller compactor using combined smooth/knurled rolls and an integrated 1.0 mm mesh milling screen with pocketed rotor and paddle agitator. The roller compactor was operated with a roll gap of 2 mm, roll pressure of 4.5 kNcm, roll speed of 2 rpm, granulation speed of 80/80 (CW/CCW) rpm, and oscillation of 330/360 (CW/CCW) degrees.

Blending:

The roller compacted granules were blended with microcrystalline cellulose using a bin blender. The blending time was 4 minutes. Magnesium stearate was added to bin and further blended for an additional 2 minutes.

Compression:

The compression blend was compressed into tablets using a Riva Piccola rotary tablet press. The weight of the tablets for a dose of 100 mg of substantially amorphous Compound I, 25.0 mg of substantially amorphous Compound II, and 75 mg of substantially amorphous Compound III was 521 mg.

Coating:

The core tablets were film coated using an Ohara tablet film coater. The film coat suspension was prepared by adding the coating material to purified water. The required amount of film coating suspension (3% of the tablet weight) was sprayed onto the tablets to achieve the desired weight gain.

TABLE 10
Tablet “B” Comprising 100 mg Compound I,
25.0 mg Compound II and 75 mg Compound III.

| | | Amount per |
|---|---|---|
| | Ingredient | tablet (mg) |
| Intra-granular | Compound I SDD (80 wt % | 125.0 |
| | Compound I and 20 wt % | |
| | HPMCAS) | |
| | Compound II SDD (80 wt % | 31.2 |
| | Compound II and 20 wt % | |
| | HPMC) | |
| | Compound III SDD (80 wt % | 93.8 |
| | Compound III, 19.5 wt % | |
| | HPMCAS, and 0.5 wt % | |
| | sodium lauryl sulfate) | |
| | Croscarmellose Sodium | 18.8 |
| | Total | 268.8 |
| Extra-granular | Microcrystalline cellulose | 143.8 |
| | Magnesium Stearate | 4.2 |
| | Total | 148.0 |
| Total uncoated Tablet | | 416.8 |
| Film coat | Opadry | 12.5 |
| Total coated Tablet | | 429.3 |

### Example 7: Preparation of Tablet Formulation C

Screening/Weighing:

The solid dispersion comprising 80 wt % substantially amorphous Compound I and 20 wt % HPMCAS as shown in Example 4, the solid dispersion comprising 80 wt % substantially amorphous Compound II and 20 wt % HPMC (see PCT Publication No. WO 2015/160787, the entire contents are incorporated herein by reference), and excipients (see Table 11) are screened prior to or after weigh-out. Screen sizes used are mesh #20 for all components except magnesium stearate which may use mesh #60.

Blending:

The solid dispersion comprising substantially amorphous Compound I, the solid dispersion comprising substantially amorphous Compound II, and microcrystalline cellulose and croscarmellose sodium are blended. The blending may be performed using a bin blender. The components may be blended for 5 minutes.

Dry Granulation:

The blend may be processed in a similar manner as above.

Blending:

The roller compacted granules may be blended with the solid dispersion comprising 80 wt % substantially amorphous Compound III, 19.5 wt % HPMCAS and 0.5 wt % sodium lauryl sulfate (see WO 2015/160787), and microcrystalline cellulose using a bin blender. The blending time may be 4 minutes.

Compression:

The compression blend may be compressed into tablets using a Riva Piccola rotary tablet press. The weight of the tablets for a dose of 150 mg of substantially amorphous Compound I, 25.0 mg of substantially amorphous Compound II, and 75 mg of substantially amorphous Compound III may be 855 mg.

Coating:

The core tablets may optionally be film coated using an Ohara tablet film coater. The film coat suspension may be prepared by adding the coating material to purified water. The required amount of film coating suspension (3% of the tablet weight) may be sprayed onto the tablets to achieve the desired weight gain.

TABLE 11
Tablet “C” Comprising 150 mg Compound I,
25.0 mg Compound II and 75 mg Compound III.

| | | Amount per |
|---|---|---|
| | Ingredient | tablet (mg) |
| Intra-granular | Compound I SDD (80 wt % | 187.5 |
| | Compound I and 20 wt % | |
| | HPMCAS) | |
| | Compound II SDD (80 wt % | 31.3 |
| | Compound II and 20 wt % | |
| | HPMC) | |
| | Microcrystalline cellulose | 137.5 |
| | Croscarmellose Sodium | 28.1 |
| | Total | 384.4 |
| Extra-granular | Compound III SDD (80 wt % | 94.2 |
| | Compound III, 19.5 wt % | |
| | HPMCAS, and 0.5 wt % | |
| | sodium lauryl sulfate) | |
| | Microcrystalline cellulose | 376.7 |
| | Total | 470.9 |
| Total uncoated Tablet | | 855.3 |

### Example 8: Preparation of Tablet Formulation D

Screening/Weighing:

The solid dispersion comprising 80 wt % substantially amorphous Compound II and 20 wt % HPMC (see PCT Publication No. WO 2015/160787, the entire contents are incorporated herein by reference), with the solid dispersion comprising 80 wt % substantially amorphous Compound III, 19.5 wt % HPMCAS and 0.5 wt % sodium lauryl sulfate (see WO 2015/160787), and excipients (see Table 12) are screened prior to or after weigh-out. Screen sizes used were mesh #20 for all components except magnesium stearate which may use mesh #60.

Blending:

The solid dispersion comprising substantially amorphous Compound II, the solid dispersion comprising substantially amorphous Compound III, and croscarmellose sodium and microcrystalline cellulose are blended. The blending may be performed using a bin blender. The components may be blended for 5 minutes.

Dry Granulation:

The blend may be granulated using procedures similar to those above.

Blending:

The roller compacted granules may be blended with the solid dispersion comprising 80 wt % substantially amorphous Compound I and 20 wt % HPMCAS as shown in Example 4, and microcrystalline cellulose and magnesium stearate using a bin blender. The blending time may be 4 minutes.

Compression:

The compression blend may be compressed into tablets using a Riva Piccola rotary tablet press. The weight of the tablets for a dose of 150 mg of substantially amorphous Compound I, 25.0 mg of substantially amorphous Compound II, and 75 mg of substantially amorphous Compound III may be 625 mg.

Coating:

The core tablets may optionally be film coated using an Ohara tablet film coater. The film coat suspension may be prepared by adding the coating material to purified water. The required amount of film coating suspension (3% of the tablet weight) may be sprayed onto the tablets to achieve the desired weight gain.

TABLE 12
Tablet “D” Comprising 150 mg Compound I,
25.0 mg Compound II and 75 mg Compound III.

| | | Amount per |
|---|---|---|
| | Ingredient | tablet (mg) |
| Intra-granular | Compound II SDD (80 wt % | 31.2 |
| | Compound II and 20 wt % | |
| | HPMC) | |
| | Compound III SDD (80 wt % | 93.7 |
| | Compound III, 19.5 wt % | |
| | HPMCAS, and 0.5 wt % | |
| | sodium lauryl sulfate) | |
| | Microcrystalline cellulose | 143.6 |
| | Croscarmellose Sodium | 37.4 |
| | Total | 305.9 |
| Extra-granular | Compound I SDD (80 wt % | 187.5 |
| | Compound I and 20 wt % | |
| | HPMCAS) | |
| | Microcrystalline cellulose | 125.0 |
| | Magnesium Stearate | 6.3 |
| | Total | 318.8 |
| Total uncoated Tablet | | 624.7 |

### Example 9. Two-Stage Dissolution Testing

The two stage dissolution testing for solid dosage formulations was carried out in a USP Apparatus II (paddle) that was coupled with an autosampler.

Each of Formulations A-J were introduced to separate dissolution vessels containing 250 mL Dressman Fed State Simulated Gastric Fluid (FeSSGF) at 37o C with paddle speed at 75RPM. A sample was taken manually at 15 minutes time point using a cannula with a 10 μm PVDF filter attached to it, and the sample was analyzed by HPLC. The dissolution apparatus was paused at this time and 650 mL of pre-heated Fed State Simulated Intestinal Fluid (FeSSIF) (pH adjusted to 7.2, at 37o C) was introduced to vessels containing FeSSGF. An autosampler was enabled and dissolution was being initiated for the second stage using the same conditions as the first stage (FeSSGF).

Samples were collected via autosampler probes with 10 μm PVDF filters attached to it, and transferred to HPLC vials for analyses. pH of final media was 6.8.

| | |
|---|---|
| Component | Amount per 1 L |

Dressman, FeSSIF

| | | |
|---|---|---|
| Sodium Hydroxide Pellets | 8.0 | g |
| Citric Acid Monohydrate | 16.1 | g |
| Sodium Chloride | 12.0 | g |
| Sodium Taurocholate | 8.1 | g |
| Lecithin (L-Alpha-Phosphatidyl Choline) | 3.1 | g |

| | |
|---|---|
| 1.0N HCl | pH adjustment to 7.2 |
| Distilled Water | QS to 1.0 L |

Dressman, FeSSGF

| | | |
|---|---|---|
| 1.0N HCl | 10.0 | mL |
| Sodium Lauryl Sulfate | 2.5 | g |
| Sodium Chloride | 2.0 | g |
| Whole Milk | 300 | mL |

| | |
|---|---|
| Distilled Water | QS to 1.0 L |

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A single tablet comprising a first solid dispersion, a second solid dispersion, and a third solid dispersion,
(a) wherein the first solid dispersion comprises 50 mg to 300 mg of Compound I: 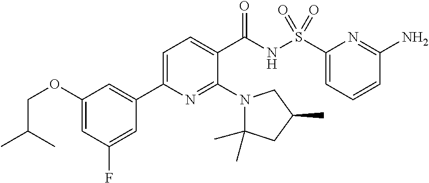 [SMILES: CC(C)COc1cc(F)cc(-c2ccc(C(=O)NS(=O)(=O)c3cccc(N)n3)c(N3C[C@@H](C)CC3(C)C)n2)c1]
and 10 wt % to 60 wt % of a polymer relative to the total weight of the first solid dispersion;
(b) wherein the second solid dispersion comprises 10 mg to 50 mg of Compound II: 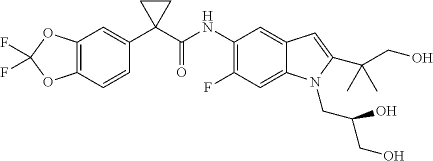 [SMILES: CC(C)(CO)c1cc2cc(NC(=O)C3(c4ccc5c(c4)OC(F)(F)O5)CC3)c(F)cc2n1C[C@@H](O)CO]
and 10 wt % to 30 wt % of a polymer relative to the total weight of the second solid dispersion; and
(c) wherein the third solid dispersion comprises 25 mg to 200 mg of Compound III: 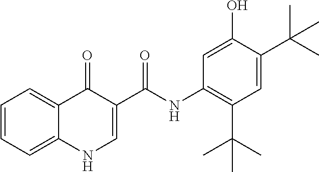 [SMILES: CC(C)(C)c1cc(C(C)(C)C)c(NC(=O)c2c[nH]c3ccccc3c2=O)cc1O]
and 10 wt % to 30 wt % of a polymer relative to the total weight of the third solid dispersion.

2. A single tablet comprising:
(a) 20 wt % to 50 wt % of a first solid dispersion relative to the total weight of the tablet;
(b) 10 wt % to 30 wt % of a second solid dispersion relative to the total weight of the tablet; and
(c) 3 wt % to 10 wt % of a third solid dispersion relative to the total weight of the tablet;
wherein the first solid dispersion comprises 40 wt % to 90 wt % of Compound I: 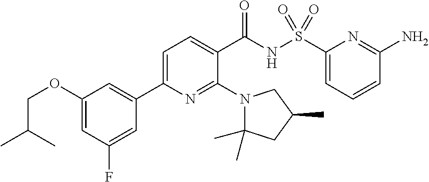 [SMILES: CC(C)COc1cc(F)cc(-c2ccc(C(=O)NS(=O)(=O)c3cccc(N)n3)c(N3C[C@@H](C)CC3(C)C)n2)c1]
and 10 wt % to 60 wt % of a polymer relative to the total weight of the first solid dispersion;
wherein the second solid dispersion comprises 70 wt % to 90 wt % of Compound II: 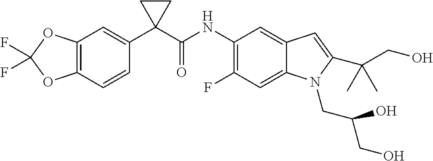 [SMILES: CC(C)(CO)c1cc2cc(NC(=O)C3(c4ccc5c(c4)OC(F)(F)O5)CC3)c(F)cc2n1C[C@@H](O)CO]
and 10 wt % to 30 wt % of a polymer relative to the total weight of the second solid dispersion; and
wherein the third solid dispersion comprises 70 wt % to 90 wt % of Compound III: 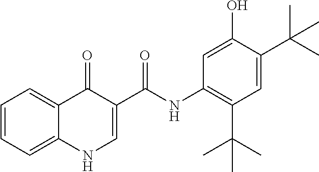 [SMILES: CC(C)(C)c1cc(C(C)(C)C)c(NC(=O)c2c[nH]c3ccccc3c2=O)cc1O]
and 10 wt % to 30 wt % of a polymer relative to the total weight of the third solid dispersion.

3. The single tablet of claim 1 or claim 2, wherein the polymer in the first solid dispersion is present in 10 wt % to 50 wt %, 10 wt % to 40 wt %, or 10 wt % to 30 wt %, relative to the total weight of the first solid dispersion.

4. The single tablet of claim 1 or claim 2, wherein the polymer in the first solid dispersion is present in 15 wt % to 25 wt % relative to the total weight of the first solid dispersion.

5. The single tablet of claim 1 or claim 2, wherein the polymer in the first solid dispersion is present in 20 wt % relative to the total weight of the first solid dispersion.

6. The single tablet of any one of claims 1-5, wherein at least one of the first, second, and third solid dispersions is a spray-dried dispersion.

7. The single tablet of any one of claims 1-5, wherein each of the first, second, and third solid dispersions is a spray-dried dispersion.

8. The single tablet of any one of claims 1-7, wherein each of said polymers in the first solid dispersion, second solid dispersion, and third solid dispersion comprises one or more polymers independently chosen from cellulose-based polymers, polyoxyethylene-based polymers, polyethylene-propylene glycol copolymers, vinyl-based polymers, PEO-polyvinyl caprolactam-based polymers, and polymethacrylate-based polymers.

9. The single tablet of claim 8,
wherein the cellulose-based polymer is chosen from a methylcellulose, a hydroxypropyl methylcellulose (hypromellose), a hypromellose phthalate (HPMC-P), and a hypromellose acetate succinate;
wherein the polyoxyethylene-based polymer or polyethylene-propylene glycol copolymer is chosen from a polyethylene glycol and a poloxamer;
wherein the vinyl-based polymer is a polyvinylpyrrolidine;
wherein the PEO-polyvinyl caprolactam-based polymer is a polyethylene glycol, polyvinyl acetate and polyvinylcaprolactam-based graft copolymer; and
wherein the polymethacrylate-based polymer is a poly(methacrylic acid, ethyl acrylate) (1:1) or a dimethylaminoethyl methacrylate-methylmethacrylate copolymer.

10. The single tablet of claim 9, wherein the cellulose-based polymer is a hypromellose acetate succinate and a hypromellose, or a combination of hypromellose acetate succinate and a hypromellose.

11. The single tablet of claim 10, wherein the cellulose-based polymer is chosen from hypromellose E15, hypromellose acetate succinate L, and hypromellose acetate succinate H.

12. The single tablet of claim 10, wherein the polyoxyethylene-based polymer or polyethylene-propylene glycol copolymer is chosen from polyethylene glycol 3350 and poloxamer 407.

13. The single tablet of claim 10, wherein the vinyl-based polymer is chosen from polyvinylpyrrolidine K30 and polyvinylpyrrolidine VA 64.

14. The single tablet of claim 10, wherein the polymethacrylate polymer is chosen from Eudragit L100-55 and Eudragit E PO.

15. The single tablet of claim 8, wherein said polymer for the first solid dispersion is chosen from a hypromellose acetate succinate and a hypromellose, and a combination thereof; said polymer for the second solid dispersion is a hypromellose; and said polymer for the third solid dispersion is a hypromellose acetate succinate.

16. The single tablet of claim 8, wherein said polymer for the first solid dispersion is a hypromellose acetate succinate; said polymer for the second solid dispersion is hypromellose; and said polymer for the third solid dispersion is a hypromellose acetate succinate.

17. The single tablet of claim 8, wherein said polymer for the first solid dispersion is chosen from hydroxypropyl methylcellulose (HPMC) E15, hypromellose acetate succinate L, hypromellose acetate succinate H, and a combination thereof; said polymer for the second solid dispersion is HPMC E15; and said polymer for the third solid dispersion is hypromellose acetate succinate H.

18. The single tablet of claim 8, wherein said polymer for the first solid dispersion is hypromellose acetate succinate H; said polymer for the second solid dispersion is HPMC E15; and said polymer for the third solid dispersion is hypromellose acetate succinate H.

19. The single tablet of claim 8, wherein said polymer for the first solid dispersion is hypromellose acetate succinate HG; said polymer for the second solid dispersion is HPMC E15; and said polymer for the third solid dispersion is hypromellose acetate succinate HG.

20. The single tablet of any one of claims 1-19, wherein the first solid dispersion comprises 50 mg to 600 mg of Compound I.

21. The single tablet of any one of claims 1-19, wherein the first solid dispersion comprises 50 mg to 200 mg, 75 mg to 200 mg, 50 mg, 75 mg, 100 mg, 150 mg, 200 mg, or 300 mg of Compound I.

22. The single tablet of any one of claims 1-19, wherein the first solid dispersion comprises 100 mg of Compound I.

23. The single tablet of any one of claims 1-19, wherein the first solid dispersion comprises 150 mg of Compound I.

24. The single tablet of any one of claims 1-23, wherein the second solid dispersion comprises 15 mg to 50 mg of Compound II.

25. The single tablet of any one of claims 1-23, wherein the second solid dispersion comprises 20 mg to 35 mg of Compound II.

26. The single tablet of any one of claims 1-23, wherein the second solid dispersion comprises 10 mg to 30 mg, 15 mg to 30 mg, or 20 mg to 30 mg of Compound II.

27. The single tablet of any one of claims 1-24, wherein the third solid dispersion comprises 50 mg to 200 mg of Compound III.

28. The single tablet of any one of claims 1-25, wherein the third solid dispersion comprises 50 mg to 175 mg, 50 mg to 100 mg, or 50 mg to 80 mg of Compound III.

29. The single tablet of any one of claims 1-19, wherein:
the first solid dispersion comprises 50 mg to 200 mg of Compound I:
the second solid dispersion comprises 15 mg to 50 mg of Compound II: and
the third solid dispersion comprises 50 mg to 200 mg of Compound.

30. The single tablet of any one of claims 1-19, wherein:
the first solid dispersion comprises 75 mg to 200 mg of Compound I:
the second solid dispersion comprises 10 mg to 30 mg of Compound II: and
the third solid dispersion comprises 50 mg to 100 mg of Compound.

31. The single tablet of any one of claims 1-19, wherein:
the first solid dispersion comprises 100 mg to 200 mg of Compound I:
the second solid dispersion comprises 20 mg to 30 mg of Compound II: and
the third solid dispersion comprises 50 mg to 80 mg of Compound III.

32. The single tablet of any one of claims 1-19, wherein Compounds I, II, and III are in a weight ratio of Compound I:Compound II:Compound III 4 to 6:1:3 to 5.

33. The single tablet of any one of claims 1-19, wherein Compounds I, II, and III are in a weight ratio of Compound I:Compound II:Compound III 4 to 6:1:3.

34. The single tablet of any one of claims 1-33, comprising one or more excipients chosen from a filler, a disintegrant, a surfactant, and a lubricant.

35. The single tablet of claim 34, wherein the filler is chosen from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.

36. The single tablet of claim 34, wherein the disintegrant is chosen from croscarmellose sodium, sodium starch glycolate, crospovidone, corn or pre-gelatinized starch, sodium carboxymethyl cellulose, calcium carboxymethyl cellulose, and microcrystalline cellulose.

37. The single tablet of claim 34, wherein the lubricant is chosen from magnesium stearate, sodium stearyl fumarate, calcium stearate, sodium stearate, stearic acid, and talc; and wherein the surfactant is chosen from sodium lauryl sulfate, poloxamers, docusate sodium, PEGs and PEG derivatives.

38. The single tablet of any one of claims 1-37, wherein each of Compounds I, II and III is independently substantially amorphous.

39. The single tablet of claim 1 or claim 2, wherein:
the second solid dispersion comprises 70 wt % to 85 wt % of Compound II relative to the total weight of the second solid dispersion, and the polymer is hydroxypropyl methylcellulose in an amount of 15 wt % to 30 wt % relative to the total weight of the second solid dispersion; and
the third solid dispersion comprises 70 wt % to 85 wt % of Compound III relative to the total weight of the third solid dispersion, and the polymer is hypromellose acetate succinate in an amount of 15 wt % to 30 wt % relative to the total weight of the second solid dispersion.

40. The single tablet of claim 1 or claim 2, wherein:
the second solid dispersion comprises 70 wt % to 85 wt % of Compound II relative to the total weight of the second solid dispersion, and the polymer is hydroxypropyl methylcellulose in an amount of 15 wt % to 30 wt % relative to the total weight of the second solid dispersion; and
the third solid dispersion comprises 80 wt % of Compound III relative to the total weight of the third solid dispersion, and the polymer is hypromellose acetate succinate in an amount of 15 wt % to 20 wt % relative to the total weight of the second solid dispersion.

41. The single tablet of any one of claims 1-40, wherein the first solid dispersion comprises 50 wt % to 90 wt % of Compound I.

42. The single tablet of any one of claims 1-40, wherein the first solid dispersion comprises 60 wt % to 90 wt % of Compound I.

43. The single tablet of any one of claims 1-40, wherein the first solid dispersion comprises 70 wt % to 90 wt % of Compound I.

44. The single tablet of any one of claims 1-40, wherein the first solid dispersion comprises 75 wt % to 85 wt % of Compound I.

45. The single tablet of any one of claims 1-40, wherein the first solid dispersion comprises 80 wt % of Compound I.

46. The single tablet of any one of claims 1-45, wherein the second solid dispersion comprises 75 wt % to 85 wt % of Compound II.

47. The single tablet of any one of claims 1-45, wherein the second solid dispersion comprises 80 wt % of Compound II.

48. The single tablet of any one of claims 1-47, wherein the third solid dispersion comprises 75 wt % to 85 wt % of Compound III.

49. The single tablet of any one of claims 1-47, wherein the third solid dispersion comprises 80 wt % of Compound III.

50. The single tablet of any one of claims 1-49, comprising one or more excipients chosen from a filler, a disintegrant, a surfactant, and a lubricant.

51. The single tablet of claim 40, wherein the filler is chosen from microcrystalline cellulose, silicified microcrystalline cellulose, lactose, dicalcium phosphate, mannitol, copovidone, hydroxypropyl cellulose, hypromellose, methyl cellulose, ethyl cellulose, starch, Maltodextrin, agar, guar gum, and pullulan.

52. The single tablet of claim 40, wherein the disintegrant is chosen from croscarmellose sodium, sodium starch glycolate, crospovidone, corn or pre-gelatinized starch, sodium carboxymethyl cellulose, calcium carboxymethyl cellulose, and microcrystalline cellulose.

53. The single tablet of claim 40, wherein the lubricant is chosen from magnesium stearate, sodium stearyl fumarate, calcium stearate, sodium stearate, stearic acid, and talc, and wherein the surfactant is chosen from sodium lauryl sulfate, poloxamers, docusate sodium, PEGs and PEG derivatives.

54. The single tablet of claim 1 or claim 2, comprising an intra-granular part and extra-granular part,
(a) wherein the intra-granular part comprises:
the first solid dispersion comprising said Compound I in 30 wt % to 40 wt % relative to the total weight of the tablet;
the second solid dispersion comprising said Compound II in 4 wt % to 8 wt % relative to the total weight of the tablet;
the third solid dispersion comprising said Compound III in 15 wt % to 20 wt % relative to the total weight of the tablet;
a disintegrant in 2 wt % to 6 wt % relative to the total weight of the tablet; and
(b) wherein the extra-granular part comprises:
a filler in 30 wt % to 40 wt % relative to the total weight of the tablet; and
a lubricant in 0.5 wt % to 1.5 wt % relative to the total weight of the tablet.

55. The single tablet of claim 1 or claim 2, comprising an intra-granular part and extra-granular part,
(a) wherein the intra-granular part comprises:
the first solid dispersion comprising said Compound I in 36 wt % relative to the total weight of the tablet;
the second solid dispersion comprising said Compound II in 6 wt % relative to the total weight of the tablet;
the third solid dispersion comprising said Compound III in 18 wt % relative to the total weight of the tablet; and
a disintegrant in 4 wt % to 5 wt % relative to the total weight of the tablet; and
(b) wherein the extra-granular part comprises:
a filler in 34 wt % to 35 wt % relative to the total weight of the tablet; and
a lubricant in 1 wt % relative to the total weight of the tablet.

56. A single tablet comprising an intra-granular portion and an extra-granular portion, wherein either of the intra-granular portion or extra-granular position are comprised of a first solid dispersion comprising Compound I and a polymer, a second solid dispersion comprising Compound II and a polymer, and a third solid dispersion comprising Compound III and a polymer.

57. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 17 mg-20 mg croscarmellose sodium; and
(b) wherein the extra-granular part comprises:
(i) 286 mg-289 mg microcrystalline cellulose; and
(ii) 5 mg-7 mg magnesium stearate.

58. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, relative to the total weight of the third solid dispersion, wherein the polymer is HPMCAS, and 0.5 wt % of sodium lauryl sulfate; and
(iv) 18 mg-21 mg croscarmellose sodium; and
(b) wherein the extra-granular part comprises:
(i) 108 mg-111 mg microcrystalline cellulose; and
(ii) 4 mg-6 mg magnesium stearate.

59. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 36 mg-39 mg croscarmellose sodium; and
(b) wherein the extra-granular part comprises:
(i) 267 mg-270 mg microcrystalline cellulose; and
(ii) 5-8 mg magnesium stearate.

60. The single tablet of claim 46,
(a) wherein the intra-granular part comprises:
(i) 186 mg-189 mg of a first solid dispersion of 80 wt % Compound I and 20 wt % of a polymer, wherein the polymer is HPMCAS, relative to the total weight of the first solid dispersion;
(ii) 30 mg-33 mg of a second solid dispersion of 80 wt % Compound II and 20 wt % of a polymer, wherein the polymer is HPMC, relative to the total weight of the second solid dispersion;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 27 mg-30 mg croscarmellose sodium; and
(b) wherein the extra-granular part comprises:
(i) 277 mg-280 mg microcrystalline cellulose; and
(ii) 5 mg-8 mg magnesium stearate.

61. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 22 mg-25 mg croscarmellose sodium; and
(b) wherein the extra-granular part comprises:
(i) 273 mg-276 mg microcrystalline cellulose; and
(ii) 5 mg-8 mg magnesium stearate.

62. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 22 mg-25 mg croscarmellose sodium; and
(b) wherein the extra-granular part comprises:
(i) 178 mg-181 mg microcrystalline cellulose; and
(ii) 4 mg-7 mg magnesium stearate.

63. The single tablet of claim 46,
(a) wherein the intra-granular part comprises:
(i) 123 mg-127 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 17 mg-20 mg croscarmellose sodium; and
(b) wherein the extra-granular part comprises:
(i) 142 mg-145 mg microcrystalline cellulose; and
(ii) 3 mg-6 mg magnesium stearate.

64. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion;
(iv) 152 mg-155 mg microcrystalline cellulose; and
(v) 27 mg-30 mg croscarmellose sodium; and
(b) wherein the extra-granular part comprises:
(i) 123 mg-127 mg microcrystalline cellulose; and
(ii) 5 mg-8 mg magnesium stearate.

65. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 29 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iv) 152 mg-155 mg microcrystalline cellulose; and
(b) wherein the extra-granular part comprises:
(i) 123 mg-127 mg microcrystalline cellulose;
(ii) 5 mg-8 mg magnesium stearate; and
(iii) 27 mg-30 mg croscarmellose sodium.

66. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 29 mg-33 mg of a second solid dispersion of 80 wt % Compound II and 20 wt % of a polymer, relative to the total weight of the second solid dispersion, wherein the polymer is HPMC;
(iii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion;
(iv) 36 mg-39 mg croscarmellose sodium; and
(v) 5 mg-8 mg sodium lauryl sulfate; and
(b) wherein the extra-granular part comprises:
(i) 261 mg-264 mg microcrystalline cellulose; and
(ii) 5 mg-8 mg magnesium stearate.

67. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 136 mg-139 mg microcrystalline cellulose; and
(iv) 27 mg-30 mg croscarmellose sodium; and
(b) wherein the extra-granular part comprises:
(i) 93 mg-96 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(ii) 375 mg-378 mg microcrystalline cellulose.

68. The single tablet of claim 46,
(a) wherein the intra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 30 mg-33 mg of a second solid dispersion of 80 wt % Compound II and 20 wt % of a polymer, relative to the total weight of the second solid dispersion, wherein the polymer is HPMC;
(iii) 136 mg-139 mg microcrystalline cellulose; and
(iv) 27 mg-30 mg croscarmellose sodium; and
(b) wherein the extra-granular part comprises:
(i) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(ii) 280 mg-283 mg microcrystalline cellulose.

69. The single tablet of claim 46,
(a) wherein the intra-granular part comprises:
(i) 30 mg-33 mg of a second solid dispersion of 80 wt % Compound II and 20 wt % of a polymer, relative to the total weight of the second solid dispersion, wherein the polymer is HPMC;
(ii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion;
(iii) 36 mg-39 mg croscarmellose sodium; and
(iv) 142 mg-145 mg microcrystalline cellulose; and
(b) wherein the extra-granular part comprises:
(i) 186 mg-189 mg of a first solid dispersion of 80 wt % Compound I and 20 wt % of a polymer, wherein the polymer is HPMCAS, relative to the total weight of the first solid dispersion;
(ii) 123 mg-127 mg microcrystalline cellulose; and
(iii) 5 mg-8 mg magnesium stearate.

70. The single tablet of claim 46,
(a) wherein the intra-granular part comprises:
(i) 30 mg-33 mg of a second solid dispersion of 80 wt % Compound II and 20 wt % of a polymer, relative to the total weight of the second solid dispersion, wherein the polymer is HPMC;
(ii) 92 mg-95 mg of a third solid dispersion of 80 wt % Compound III, 19.5 wt % of a polymer, wherein the polymer is HPMCAS, and 0.5 wt % of sodium lauryl sulfate, relative to the total weight of the third solid dispersion; and
(iii) 36 mg-39 mg croscarmellose sodium; and
(b) wherein the extra-granular part comprises:
(i) 186 mg-189 mg of a first solid dispersion of 80 wt % Compound I and 20 wt % of a polymer, relative to the total weight of the first solid dispersion, wherein the polymer is HPMCAS;
(ii) 267 mg-270 mg microcrystalline cellulose; and
(iii) 5 mg-8 mg magnesium stearate.

71. A method of treating cystic fibrosis in a patient comprising orally administering to the patient the single tablet of any one of claims 1-86.

72. The method of claim 71, wherein the single tablet is administered once daily.

73. The method of claim 71, wherein the single tablet is administered twice daily.

74. The method of claim 71, wherein two tablets are administered once daily.

75. The method of claim 71, wherein two tablets are administered two times daily.

76. The method according to any one of claims 71-75, wherein said patient has cystic fibrosis is chosen from patients with F508del/minimal function genotypes, patients with F508del/F508del genotypes, patients with F508del/gating genotypes, and patients with F508del/residual function genotypes.

77. The method of claim 76, wherein the patient with a F508del/minimal function genotype has a minimal function mutation chosen from:
Mutation

| | | | | |
|---|---|---|---|---|
| S4X | C276X | G542X | R792X | E1104X |
| G27X | Q290X | G550X | E822X | R1158X |
| Q39X | G330X | Q552X | W846X | R1162X |
| W57X | W401X | R553X | Y849X | S1196X |
| E60X | Q414X | E585X | R851X | W1204X |
| R75X | S434X | G673X | Q890X | L1254X |
| E92X | S466X | Q685X | S912X | S1255X |
| Q98X | S489X | R709X | Y913X | W1282X |
| Y122X | Q493X | K710X | W1089X | Q1313X |
| E193X | W496X | L732X | Y1092X | E1371X |
| L218X | C524X | R764X | W1098X | Q1382X |
| Q220X | Q525X | R785X | R1102X | Q1411X |
| 185+1G→T | 711+5G→A | 1717−8G→A | 2622+1G→A | 3121−1G→A |
| 296+1G→A | 712−1G→T | 1717−1G→A | 2790−1G→C | 3500−2A→G |
| 405+1G→A | 1248+1G→A | 1811+1G→C | 3040G→C | 3600+2insT |
| 405+3A→C | 1249−1G→A | 1811+1.6kbA→G | (G970R) | 3850−1G→A |
| 406−1G→A | 1341+1G→A | 1812−1G→A | 3120G→A | 4005+1G→A |
| 621+1G→T | 1525−2A→G | 1898+1G→A | 3120+1G→A | 4374+1G→T |
| 711+1G→T | 1525−1G→A | 1898+1G→C | 3121−2A→G | |
| 182delT | 1119delA | 1782delA | 2732insA | 3876delA |
| 306insA | 1138insG | 1824delA | 2869insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA→G ^{a} | 2957delT | 4021dupT |
| 444delA | 1259insA | 2184delA | 3007delG | 4040delA |
| 457TAT→G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTa | 2711delT | 3821delT | |
| | | |
|---|---|---|
| CFTRdele2,3 | 1461ins4 | 2991del32 |
| CFTRdele22,23 | 1924del7 | 3199del6^{b} |
| 124del23bp | 2055del9→A | 3667ins4 |
| 852del22 | 2105-2117del13insAGAAA | 4010del4 |
| 991del5 | 2721del11 | 4209TGTT→AA |
| | | | |
|---|---|---|---|
| A46D^{b} | V520F | Y569D^{b} | N1303K |
| | | |
|---|---|---|
| G85E | A559T^{b} | L1065P |
| R347P | R560T | R1066C |
| L467P^{b} | R560S | L1077P^{b} |
| I507del | A561E | M1101K |

78. The method of claim 76, wherein the patient with a F508del/gating genotype has a gating mutation chosen from G178R, S549N, S549R, G551D, G551S, G1244E, S1251N, S1255P, and G1349A.

79. The method of claim 76, wherein the patient with a F508del/residual function genotype has a residual function mutation chosen from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.
